SEQUENCE LISTING (35 sequences)

SEQ ID NO: 1 - protein, 115 aa, synthetic construct
EVQLVESGGGLVQPGGSLRLSCAASGITFSRSVMGWFRQAPGKLRELVAGITSDDNTDYADSVKGRFTISRDNSKNTLYLQMNSLRAEDTAVYYCNILPDSRLGWGQGTLVTVSS

SEQ ID NO: 2 - protein, 123 aa, synthetic construct [sequence omitted]

SEQ ID NO: 3 - protein, 107 aa, synthetic construct [sequence omitted]

SEQ ID NO: 4 - protein, 118 aa, synthetic construct
EVQLVESGGGLVKPGGSLKLSCTASGFTFSSYVMSWVRQTPEKRLEWVATINDGGSYTYYPDNLKGRFTISRDNAKNNLYLQMSHLRSEDTARYYCSVLRRGNAMDYWGQGTSVTVSS

SEQ ID NO: 5 - protein, 113 aa, synthetic construct
DIVMTQSPSSLTATARERVTMSCKSSQSLLNSGNQKNYLTWYQQKPGQPPKLLIYWASTRESGVPDRFTGSGSGTDFTLTVSSVQAEDLAVYYCQNAYFYPFTFGSGTKLEIK

SEQ ID NO: 6 - protein, 211 aa, synthetic construct
TGSDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVLSPVNRDLKTQSGSEMKKFLSTLTIDGVTRSDQGLYTCAASSGLMTKKNSTFVRVHEK

SEQ ID NO: 7 - protein, 330 aa, synthetic construct [sequence omitted]

SEQ ID NO: 8 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLASKLTVDKSRWQQGNVFSCSVMHEALHNHYTQKSLSLSPGK

SEQ ID NO: 10 - protein, 107 aa, synthetic construct [sequence omitted]

SEQ ID NO: 11 - protein, 5 aa, synthetic construct
GGGGS

SEQ ID NO: 12 - protein, 220 aa, synthetic construct
DIVMTQSPSSLTATARERVTMSCKSSQSLLNSGNQKNYLTWYQQKPGQPPKLLIYWASTRESGVPDRFTGSGSGTDFTLTVSSVQAEDLAVYYCQNAYFYPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - protein, 350 aa, synthetic construct
EVQLVESGGGLVQPGGSLRLSCAASGITFSRSVMGWFRQAPGKLRELVAGITSDDNTDYADSVKGRFTISRDNSKNTLYLQMNSLRAEDTAVYYCNILPDSRLGWGQGTLVTVSSGGGGSGGGGSGGGGSDIVMTQSPSSLTATARERVTMSCKSSQSLLNSGNQKNYLTWYQQKPGQPPKLLIYWASTRESGVPDRFTGSGSGTDFTLTVSSVQAEDLAVYYCQNAYFYPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 14 - protein, 355 aa, synthetic construct
DIVMTQSPSSLTATARERVTMSCKSSQSLLNSGNQKNYLTWYQQKPGQPPKLLIYWASTRESGVPDRFTGSGSGTDFTLTVSSVQAEDLAVYYCQNAYFYPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSEVQLVESGGGLVQPGGSLRLSCAASGITFSRSVMGWFRQAPGKLRELVAGITSDDNTDYADSVKGRFTISRDNSKNTLYLQMNSLRAEDTAVYYCNILPDSRLGWGQGTLVTVSS

SEQ ID NO: 15 - protein, 355 aa, synthetic construct
EVQLVESGGGLVQPGGSLRLSCAASGITFSRSVMGWFRQAPGKLRELVAGITSDDNTDYADSVKGRFTISRDNSKNTLYLQMNSLRAEDTAVYYCNILPDSRLGWGQGTLVTVSSGGGGSGGGGSGGGGSEVQLVESGGGLVKPGGSLKLSCTASGFTFSSYVMSWVRQTPEKRLEWVATINDGGSYTYYPDNLKGRFTISRDNAKNNLYLQMSHLRSEDTARYYCSVLRRGNAMDYWGQGTSVTVSSRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 16 - protein, 360 aa, synthetic construct
EVQLVESGGGLVKPGGSLKLSCTASGFTFSSYVMSWVRQTPEKRLEWVATINDGGSYTYYPDNLKGRFTISRDNAKNNLYLQMSHLRSEDTARYYCSVLRRGNAMDYWGQGTSVTVSSRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSEVQLVESGGGLVQPGGSLRLSCAASGITFSRSVMGWFRQAPGKLRELVAGITSDDNTDYADSVKGRFTISRDNSKNTLYLQMNSLRAEDTAVYYCNILPDSRLGWGQGTLVTVSS

SEQ ID NO: 17 - protein, 677 aa, synthetic construct
EVQLVESGGGLVKPGGSLKLSCTASGFTFSSYVMSWVRQTPEKRLEWVATINDGGSYTYYPDNLKGRFTISRDNAKNNLYLQMSHLRSEDTARYYCSVLRRGNAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSGGGGSTGSDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVLSPVNRDLKTQSGSEMKKFLSTLTIDGVTRSDQGLYTCAASSGLMTKKNSTFVRVHEK

SEQ ID NO: 18 - protein, 807 aa, synthetic construct
EVQLVESGGGLVQPGGSLRLSCAASGITFSRSVMGWFRQAPGKLRELVAGITSDDNTDYADSVKGRFTISRDNSKNTLYLQMNSLRAEDTAVYYCNILPDSRLGWGQGTLVTVSSGGGGSGGGGSGGGGSEVQLVESGGGLVKPGGSLKLSCTASGFTFSSYVMSWVRQTPEKRLEWVATINDGGSYTYYPDNLKGRFTISRDNAKNNLYLQMSHLRSEDTARYYCSVLRRGNAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSGGGGSTGSDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVLSPVNRDLKTQSGSEMKKFLSTLTIDGVTRSDQGLYTCAASSGLMTKKNSTFVRVHEK

SEQ ID NO: 19 - protein, 672 aa, synthetic construct
DIVMTQSPSSLTATARERVTMSCKSSQSLLNSGNQKNYLTWYQQKPGQPPKLLIYWASTRESGVPDRFTGSGSGTDFTLTVSSVQAEDLAVYYCQNAYFYPFTFGSGTKLEIKASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSGGGGSTGSDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVLSPVNRDLKTQSGSEMKKFLSTLTIDGVTRSDQGLYTCAASSGLMTKKNSTFVRVHEK

SEQ ID NO: 20 - protein, 677 aa, synthetic construct
EVQLVESGGGLVKPGGSLKLSCTASGFTFSSYVMSWVRQTPEKRLEWVATINDGGSYTYYPDNLKGRFTISRDNAKNNLYLQMSHLRSEDTARYYCSVLRRGNAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLASKLTVDKSRWQQGNVFSCSVMHEALHNHYTQKSLSLSPGGGGSGGGGSGGGGSGGGGSTGSDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVLSPVNRDLKTQSGSEMKKFLSTLTIDGVTRSDQGLYTCAASSGLMTKKNSTFVRVHEK

SEQ ID NO: 21 - protein, 807 aa, synthetic construct
EVQLVESGGGLVQPGGSLRLSCAASGITFSRSVMGWFRQAPGKLRELVAGITSDDNTDYADSVKGRFTISRDNSKNTLYLQMNSLRAEDTAVYYCNILPDSRLGWGQGTLVTVSSGGGGSGGGGSGGGGSEVQLVESGGGLVKPGGSLKLSCTASGFTFSSYVMSWVRQTPEKRLEWVATINDGGSYTYYPDNLKGRFTISRDNAKNNLYLQMSHLRSEDTARYYCSVLRRGNAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSGGGGSTGSDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVLSPVNRDLKTQSGSEMKKFLSTLTIDGVTRSDQGLYTCAASSGLMTKKNSTFVRVHEK

SEQ ID NO: 22 - protein, 711 aa, synthetic construct
EVQLVESGGGLVKPGGSLKLSCTASGFTFSSYVMSWVRQTPEKRLEWVATINDGGSYTYYPDNLKGRFTISRDNAKNNLYLQMSHLRSEDTARYYCSVLRRGNAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSGGGGSEVQLVESGGGLVQPGGSLRLSCAASGYTFTNYGMNWVRQAPCKGLEWVGWINTYTGEPTYAADFKRRFTFSLDTSKSTAYLQMNSLRAEDTAVYYCAKYPHYYGSSHWYFDVWGQGTLVTVSSGGGGSGGGGSGGGGSDIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGCGTKVEIK

SEQ ID NO: 23 - protein, 841 aa, synthetic construct
EVQLVESGGGLVQPGGSLRLSCAASGITFSRSVMGWFRQAPGKLRELVAGITSDDNTDYADSVKGRFTISRDNSKNTLYLQMNSLRAEDTAVYYCNILPDSRLGWGQGTLVTVSSGGGGSGGGGSGGGGSEVQLVESGGGLVKPGGSLKLSCTASGFTFSSYVMSWVRQTPEKRLEWVATINDGGSYTYYPDNLKGRFTISRDNAKNNLYLQMSHLRSEDTARYYCSVLRRGNAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSGGGGSEVQLVESGGGLVQPGGSLRLSCAASGYTFTNYGMNWVRQAPCKGLEWVGWINTYTGEPTYAADFKRRFTFSLDTSKSTAYLQMNSLRAEDTAVYYCAKYPHYYGSSHWYFDVWGQGTLVTVSSGGGGSGGGGSGGGGSDIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGCGTKVEIK

SEQ ID NO: 24 - protein, 706 aa, synthetic construct
DIVMTQSPSSLTATARERVTMSCKSSQSLLNSGNQKNYLTWYQQKPGQPPKLLIYWASTRESGVPDRFTGSGSGTDFTLTVSSVQAEDLAVYYCQNAYFYPFTFGSGTKLEIKASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSGGGGSEVQLVESGGGLVQPGGSLRLSCAASGYTFTNYGMNWVRQAPCKGLEWVGWINTYTGEPTYAADFKRRFTFSLDTSKSTAYLQMNSLRAEDTAVYYCAKYPHYYGSSHWYFDVWGQGTLVTVSSGGGGSGGGGSGGGGSDIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGCGTKVEIK

SEQ ID NO: 25 - protein, 711 aa, synthetic construct
EVQLVESGGGLVKPGGSLKLSCTASGFTFSSYVMSWVRQTPEKRLEWVATINDGGSYTYYPDNLKGRFTISRDNAKNNLYLQMSHLRSEDTARYYCSVLRRGNAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLASKLTVDKSRWQQGNVFSCSVMHEALHNHYTQKSLSLSPGGGGSGGGGSGGGGSGGGGSEVQLVESGGGLVQPGGSLRLSCAASGYTFTNYGMNWVRQAPCKGLEWVGWINTYTGEPTYAADFKRRFTFSLDTSKSTAYLQMNSLRAEDTAVYYCAKYPHYYGSSHWYFDVWGQGTLVTVSSGGGGSGGGGSGGGGSDIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGCGTKVEIK

SEQ ID NO: 26 - protein, 841 aa, synthetic construct
EVQLVESGGGLVQPGGSLRLSCAASGITFSRSVMGWFRQAPGKLRELVAGITSDDNTDYADSVKGRFTISRDNSKNTLYLQMNSLRAEDTAVYYCNILPDSRLGWGQGTLVTVSSGGGGSGGGGSGGGGSEVQLVESGGGLVKPGGSLKLSCTASGFTFSSYVMSWVRQTPEKRLEWVATINDGGSYTYYPDNLKGRFTISRDNAKNNLYLQMSHLRSEDTARYYCSVLRRGNAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSGGGGSEVQLVESGGGLVQPGGSLRLSCAASGYTFTNYGMNWVRQAPCKGLEWVGWINTYTGEPTYAADFKRRFTFSLDTSKSTAYLQMNSLRAEDTAVYYCAKYPHYYGSSHWYFDVWGQGTLVTVSSGGGGSGGGGSGGGGSDIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGCGTKVEIK

SEQ ID NO: 27 - protein, 711 aa, synthetic construct
EVQLVESGGGLVKPGGSLKLSCTASGFTFSSYVMSWVRQTPEKRLEWVATINDGGSYTYYPDNLKGRFTISRDNAKNNLYLQMSHLRSEDTARYYCSVLRRGNAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSGGGGSDIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGCGTKVEIKGGGGSGGGGSGGGGSEVQLVESGGGLVQPGGSLRLSCAASGYTFTNYGMNWVRQAPCKGLEWVGWINTYTGEPTYAADFKRRFTFSLDTSKSTAYLQMNSLRAEDTAVYYCAKYPHYYGSSHWYFDVWGQGTLVTVSS

SEQ ID NO: 28 - protein, 841 aa, synthetic construct
EVQLVESGGGLVQPGGSLRLSCAASGITFSRSVMGWFRQAPGKLRELVAGITSDDNTDYADSVKGRFTISRDNSKNTLYLQMNSLRAEDTAVYYCNILPDSRLGWGQGTLVTVSSGGGGSGGGGSGGGGSEVQLVESGGGLVKPGGSLKLSCTASGFTFSSYVMSWVRQTPEKRLEWVATINDGGSYTYYPDNLKGRFTISRDNAKNNLYLQMSHLRSEDTARYYCSVLRRGNAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSGGGGSDIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGCGTKVEIKGGGGSGGGGSGGGGSEVQLVESGGGLVQPGGSLRLSCAASGYTFTNYGMNWVRQAPCKGLEWVGWINTYTGEPTYAADFKRRFTFSLDTSKSTAYLQMNSLRAEDTAVYYCAKYPHYYGSSHWYFDVWGQGTLVTVSS

SEQ ID NO: 29 - protein, 706 aa, synthetic construct
DIVMTQSPSSLTATARERVTMSCKSSQSLLNSGNQKNYLTWYQQKPGQPPKLLIYWASTRESGVPDRFTGSGSGTDFTLTVSSVQAEDLAVYYCQNAYFYPFTFGSGTKLEIKASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSGGGGSDIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGCGTKVEIKGGGGSGGGGSGGGGSEVQLVESGGGLVQPGGSLRLSCAASGYTFTNYGMNWVRQAPCKGLEWVGWINTYTGEPTYAADFKRRFTFSLDTSKSTAYLQMNSLRAEDTAVYYCAKYPHYYGSSHWYFDVWGQGTLVTVSS

SEQ ID NO: 30 - protein, 711 aa, synthetic construct
EVQLVESGGGLVKPGGSLKLSCTASGFTFSSYVMSWVRQTPEKRLEWVATINDGGSYTYYPDNLKGRFTISRDNAKNNLYLQMSHLRSEDTARYYCSVLRRGNAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLASKLTVDKSRWQQGNVFSCSVMHEALHNHYTQKSLSLSPGGGGSGGGGSGGGGSGGGGSDIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGCGTKVEIKGGGGSGGGGSGGGGSEVQLVESGGGLVQPGGSLRLSCAASGYTFTNYGMNWVRQAPCKGLEWVGWINTYTGEPTYAADFKRRFTFSLDTSKSTAYLQMNSLRAEDTAVYYCAKYPHYYGSSHWYFDVWGQGTLVTVSS

SEQ ID NO: 31 - protein, 841 aa, synthetic construct
EVQLVESGGGLVQPGGSLRLSCAASGITFSRSVMGWFRQAPGKLRELVAGITSDDNTDYADSVKGRFTISRDNSKNTLYLQMNSLRAEDTAVYYCNILPDSRLGWGQGTLVTVSSGGGGSGGGGSGGGGSEVQLVESGGGLVKPGGSLKLSCTASGFTFSSYVMSWVRQTPEKRLEWVATINDGGSYTYYPDNLKGRFTISRDNAKNNLYLQMSHLRSEDTARYYCSVLRRGNAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSGGGGSDIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGCGTKVEIKGGGGSGGGGSGGGGSEVQLVESGGGLVQPGGSLRLSCAASGYTFTNYGMNWVRQAPCKGLEWVGWINTYTGEPTYAADFKRRFTFSLDTSKSTAYLQMNSLRAEDTAVYYCAKYPHYYGSSHWYFDVWGQGTLVTVSS

SEQ ID NO: 32 - protein, 344 aa, synthetic construct
EVQLVESGGGLVQPGGSLRLSCAASGITFSRSVMGWFRQAPGKLRELVAGITSDDNTDYADSVKGRFTISRDNSKNTLYLQMNSLRAEDTAVYYCNILPDSRLGWGQGTLVTVSSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 33 - protein, 447 aa, synthetic construct
EVQLVESGGGLVKPGGSLKLSCTASGFTFSSYVMSWVRQTPEKRLEWVATINDGGSYTYYPDNLKGRFTISRDNAKNNLYLQMSHLRSEDTARYYCSVLRRGNAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 34 - protein, 245 aa, synthetic construct
EVQLVESGGGLVQPGGSLRLSCAASGYTFTNYGMNWVRQAPCKGLEWVGWINTYTGEPTYAADFKRRFTFSLDTSKSTAYLQMNSLRAEDTAVYYCAKYPHYYGSSHWYFDVWGQGTLVTVSSGGGGSGGGGSGGGGSDIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGCGTKVEIK

SEQ ID NO: 35 - protein, 245 aa, synthetic construct
DIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGCGTKVEIKGGGGSGGGGSGGGGSEVQLVESGGGLVQPGGSLRLSCAASGYTFTNYGMNWVRQAPCKGLEWVGWINTYTGEPTYAADFKRRFTFSLDTSKSTAYLQMNSLRAEDTAVYYCAKYPHYYGSSHWYFDVWGQGTLVTVSS

(19) United States
(12) Patent Application Publication
(10) Pub. No.: US 2024/0327543 A1
(43) Pub. Date: Oct. 3, 2024

(54) MULTISPECIFIC ANTIGEN-BINDING PROTEIN AND USE THEREOF

(71) Applicant: SHENGHE (CHINA) BIOPHARMACEUTICAL CO., LTD., Nanjing, Jiangsu (CN)

(72) Inventors: Chong ZHOU, Nanjing (CN); Liusong YIN, Nanjing (CN); Xiaoling JIANG, Nanjing (CN)

(73) Assignee: SHENGHE (CHINA) BIOPHARMACEUTICAL CO., LTD., Nanjing, Jiangsu (CN)

(21) Appl. No.: 18/293,515

(22) PCT Filed: Jul. 27, 2022
(86) PCT No.: PCT/CN2022/108067

§ 371 Date: Jan. 30, 2024

(30) Foreign Application Priority Data
Aug. 2, 2021 (WO) PCT/CN2021/110025

(51) Int. Cl. C07K 16/46; A61P 35/00

(52) U.S. Cl. CPC C07K 16/468; A61P 35/00; C07K 2317/31; C07K 2317/522; C07K 2317/524; C07K 2317/526; C07K 2317/53; C07K 2317/569

(57) ABSTRACT

A multispecific antigen-binding protein including: (a) a first antigen-binding portion capable of recognizing the first antigen, where the first antigen is targeted to antigens associated with tumorigenesis or progression; (b) a second antigen-binding portion, where the second antigen-binding portion is an innate immune cell agonist; and (c) a third antigen-binding portion capable of recognizing the third antigen, where the third antigen regulates tumor microenvironment by regulating normal and abnormal angiogenesis. The embodiments further provide a pharmaceutical composition including the multispecific antigen-binding protein and a pharmaceutically acceptable carrier, and use of the multispecific antigen-binding protein and the pharmaceutical composition in the preparation of a drug for the treatment of cancer.

### TECHNICAL FIELD

The present invention belongs to the field of biotechnology and specifically relates to a multispecific antigen-binding protein that specifically binds to two or more different antigens or epitopes and use thereof.

### BACKGROUND

Monoclonal antibodies (mAb) have been widely used to treat a variety of human diseases, including cancer, autoimmune diseases, infectious diseases, and cardiovascular diseases. Currently, more than 30 monoclonal antibodies exist, including murine, fully humanized and chimeric antibodies, which have been approved by the FDA for therapeutic use.

Most of these antibodies are monospecific antibodies that recognize a single epitope and can be selected to activate or inhibit the activity of a target molecule through this single epitope. For example, trastuzumab, one of the best-selling anti-cancer protein therapeutics, blocks the growth of cancer cells by attaching itself to Her2 to prevent human epidermal growth factor from attaching itself to Her2. Trastuzumab also stimulates the body's own immune cells to destroy cancer cells. However, many physiological responses require cross-linking or co-joining of two or more different proteins or protein subunits to be triggered. As an example, for the activation of heteropolymeric cell-surface receptor complexes, for which activation is usually achieved through the interaction of ligands with multiple structural domains on different proteins, resulting in the proximity-associated activation of one or both receptor components.

Multispecific antibodies, which can co-engage multiple epitopes or antigens, have been designed to simultaneously modulate two or more therapeutic targets, providing enhanced therapeutic efficacy and broadened potential utility. Multispecific antibodies address multiple mechanisms of tumorigenesis and block tumor growth in multiple dimensions. The mechanisms of action of currently available antitumor drugs are divided into several aspects: (1) specific targeting of antigens related to tumor development or progression, including TSA (tumor-specific antigen) and TAA (tumor-associated antigen); (2) relieving immunosuppression and stimulating immune cell activity (such as PD1 immunosuppressant therapy, CD3 T cell engager); (3) improving the tumor microenvironment.

Currently, immune cell activity is mainly activated by activating T cell activity on the market, but the therapy for activating immune T cell has the following clinical limitations: (1) high toxicity (i.e. cytokine storm and neurotoxicity); (2) down-regulation of MHCI by cancer cells to achieve immune escape; (3) low T-cell activity in the tumor microenvironment of tumor patients; (4) limited clinical efficacy of solid tumors.

Innate immunity is a natural defense ability gradually established by humans in the long-term evolution process. It is controlled by genetic factors and has relative stability; it has a certain degree of resistance to various parasitic infections. Innate immune cells, represented by NK cells, will actively attack and eliminate various bacteria, viruses, diseased and cancerous cells that are harmful to the human body, with a broad spectrum.

γδT cells are T cells that perform innate immune functions. Activated γδT cells can induce tumor cell apoptosis through the apoptosis-inducing protein ligand pathway Fas-FasL and related apoptosis-inducing ligand receptors. γδT cells can secrete a large number of cytokines, such as interferon γ (IFN-γ), tumor necrosis factor α (TNF-α) and interleukin 2 (IL-2), which act on tumor cells and their microenvironment. γδT cells can also participate in immune regulation by secreting IL-2, IL-17 and other cytokines and chemokines.

NK cells are the first line of defence recognized by the medical communit. Compared with other anti-cancer immune cells, NK cells have a stronger and more effective effect on killing tumors and virus-infected cells. An NK cell can kill a tumor cell that is several times larger than the NK cell by releasing perforin and granzyme. Its activation is not dependent on tumor cell surface antigens, and it does not need to undergo an antigen recognition response by the immune system to identify the target of “attack”, as is the case with T cells. NK cells roam the blood vessels throughout the body exercising an immune surveillance role. They are the first to detect and rapidly activate the immune defence and immune stabilisation functions to kill diseased and cancerous cells. The killing effect of NK cells after acting on target cells can be seen in 1 hour in vitro and 4 hours in vivo. Major human NK cell activating receptors include CD16, NKG2D and natural cytotoxicity receptors (NCRs), the latter of which include NKp30, NKp44 and NKp46.

NKp30 is expressed on a variety of innate immune cells, including all resting and activated NK cells, various effector NKT cells, γδT-cells, MAIT cells. NKP30 can activate tumor-killing cells such as NK cells and γδT-cells, which in turn kill tumors. NKp30 recognizes ligands on tumor cells and dendritic cells that are highly expressed on a subset of tumor cells but not on most other normal cells. Stimulating or inhibiting the activation of NKp30 may be used to regulate the activity of NK cells, as well as to treat diseases or disorders related to NK cell activity. In particular, enhancement of NK cell activity by triggering NKp30 may be used to treat diseases or disorders characterized by insufficient NK cell activity, such as cancer and infectious diseases, while inhibition of NK cell activity by blocking NKp30 can be used to treat NK cell-mediated disorders, such as BMC allograft rejection.

Multiple mechanisms lead to dysfunction of tumor blood vessels, which in turn affects the tumor microenvironment. The most significant one is the signal imbalance mediated by angiogenesis and anti angiogenic molecules. In nonmalignant tissues, this balance is carefully maintained to ensure the normal morphology and function of blood vessels. However, during the process of cancer, this balance often leads to angiogenesis, making blood vessels immature and abnormal. In most solid tumor patients, vascular abnormalities can help the tumor evade immune system attacks. These abnormalities are caused by an increase in angiogenic factors such as VEGF and angiopoietin-2 (ANG2). The use of drugs targeting these molecules can enhance the responsiveness of immunotherapy. Part of the reason is that such drugs can normalize abnormal tumor vascular systems, increasing the infiltration of immune effector cells, transforming the immunosuppressed tumor microenvironment (TME) into an immune enhanced tumor microenvironment.

The multispecific antibody of the present invention specifically targets antigens related to tumor occurrence or development, and can specifically activate innate immune cells such as NK cells and γδT cells in the tumor microenvironment to relieve immune suppression. By bridging NK, γδT innate immune cells and tumor cells, increasing the immune synapse between NK cells, γδT innate immune cells and tumor cells, and killing tumor cells more effectively. VEGF, as a pro angiogenic factor, promotes the growth of abnormal blood vessels in the tumor microenvironment and promotes tumor progression. The multi-specific antibody of the invention binds and neutralizes free VEGF through VEGF antibody or VEGFR, blocks the signaling between VEGF and receptor, and inhibits the abnormal generation of blood vessels in the tumor microenvironment.

### SUMMARY

The present invention provides a multispecific antigen-binding protein comprising: (a) a first antigen-binding portion capable of specifically recognizing the first antigen, where the first antigen is specifically targeted to antigens associated with tumorigenesis or progression; (b) a second antigen-binding portion, where the second antigen-binding portion is an innate immune cell agonist; and (c) a third antigen-binding portion capable of specifically recognizing the third antigen, where the third antigen regulates tumor microenvironment by regulating normal and abnormal angiogenesis. The positions of the second antigen-binding portion and the third antigen-binding portion are interchangeable.

In some embodiments, the antigen specifically targeting the tumorigenesis or progression is a tumor-associated antigen (TAA) and/or a tumor-specific antigen (TSA).

In some embodiments, the innate immune cell agonist is an NK cell agonist and/or a γδT cell agonist.

In some embodiments, the second antigen-binding protein specifically recognizes the second antigen expressed on the innate immune cells, and the second antigen-binding protein activates the innate immune cells after binding to the second antigen.

In some embodiments, the third antigen is a pro-angiogenic factor.

In some embodiments, the first antigen-binding portion and/or the second antigen-binding portion and/or the third antigen-binding portion is a full-length antibody comprising two heavy chains and two light chains.

In some embodiments, the first antigen-binding portion and/or the second antigen-binding portion and/or the third antigen-binding portion is an antibody fragment comprising a heavy chain variable domain (VH) and/or a light chain variable domain (VL).

In some embodiments, the first antigen-binding portion and/or the second antigen-binding portion and/or the third antigen-binding portion is one of Fab, scFab, F(ab′)2, Fv, dsFv, scFv, VH or VL structural domain.

In some embodiments, the first antigen-binding portion and/or the second antigen-binding portion and/or the third antigen-binding portion is a single-domain antibody (VHH).

In some embodiments, the first antigen-binding portion and/or the second antigen-binding portion and/or the third antigen-binding portion is a receptor for an antigen.

In some embodiments, the second antigen-binding portion is fused to the N-terminus of one of the heavy chains of the first antigen-binding portion.

In some embodiments, the second antigen-binding portion is fused to the C-terminus of one of the heavy chains of the first antigen-binding portion.

In some embodiments, the second antigen-binding portion is fused to the N-terminus and C-terminus of one of the heavy chains of the first antigen-binding portion.

In some embodiments, the second antigen-binding portion is fused to the N-terminus of one of the heavy chains of the first antigen-binding portion, and the third antigen-binding portion is fused to the C-terminus of the same heavy chain of the first antigen-binding portion.

In some embodiments, the second antigen-binding portion is fused to the C-terminus of one of the heavy chains of the first antigen-binding portion, and the third antigen-binding portion is fused to the N-terminus of the same heavy chain of the first antigen-binding portion.

In some embodiments, the third antigen-binding portion is fused to the N-terminus of the other heavy chain of the first antigen-binding portion.

In some embodiments, the third antigen-binding portion is fused to the C-terminus of the other heavy chain of the first antigen-binding portion.

In some embodiments, the third antigen-binding portion is fused to the N-terminus and C-terminus of the other heavy chain of the first antigen-binding portion.

In some embodiments, the second antigen-binding portion is fused to the N-terminus of one of the heavy chains of the first antigen-binding portion, and the third antigen-binding portion is fused to the N-terminus of the other heavy chain of the first antigen-binding portion.

In some embodiments, the second antigen-binding portion is fused to the N-terminus of one of the heavy chains of the first antigen-binding portion, and the third antigen-binding portion is fused to the C-terminus of the other heavy chain of the first antigen-binding portion.

In some embodiments, the second antigen-binding portion is fused to the N-terminus of one of the heavy chains of the first antigen-binding portion, and the third antigen-binding portion is fused to the N-terminus and C-terminus of the other heavy chain of the first antigen-binding portion.

In some embodiments, the second antigen-binding portion is fused to the C-terminus of one of the heavy chains of the first antigen-binding portion, and the third antigen-binding portion is fused to the N-terminus of the other heavy chain of the first antigen-binding portion.

In some embodiments, the second antigen-binding portion is fused to the C-terminus of one of the heavy chains of the first antigen-binding portion, and the third antigen-binding portion is fused to the C-terminus of the other heavy chain of the first antigen-binding portion.

In some embodiments, the second antigen-binding portion is fused to the C-terminus of one of the heavy chains of the first antigen-binding portion, and the third antigen-binding portion is fused to the N-terminus and C-terminus of the other heavy chain of the first antigen-binding portion.

In some embodiments, the second antigen-binding portion is fused to the N-terminus and C-terminus of one of the heavy chains of the first antigen-binding portion, and the third antigen-binding portion is fused to the N-terminus of the other heavy chain of the first antigen-binding portion.

In some embodiments, the second antigen-binding portion is fused to the N-terminus and C-terminus of one of the heavy chains of the first antigen-binding portion, and the third antigen-binding portion is fused to the C-terminus of the other heavy chain of the first antigen-binding portion.

In some embodiments, the second antigen-binding portion is fused to the N-terminus and C-terminus of one of the heavy chains of the first antigen-binding portion, and the third antigen-binding portion is fused to the N-terminus and C-terminus of the other heavy chain of the first antigen-binding portion.

In some embodiments, the second antigen-binding portion is fused to the N-terminus of one of the heavy chains of the first antigen-binding portion, and the third antigen-binding portion is fused to the C-terminus of the same heavy chain of the first antigen-binding portion and fused to the N-terminus of the other heavy chain of the first antigen-binding portion.

In some embodiments, the second antigen-binding portion is fused to the N-terminus of one of the heavy chains of the first antigen-binding portion, and the third antigen-binding portion is fused to the C-terminus of the same heavy chain of the first antigen-binding portion and fused to the C-terminus of the other heavy chain of the first antigen-binding portion.

In some embodiments, the second antigen-binding portion is fused to the N-terminus of one of the heavy chains of the first antigen-binding portion, and the third antigen-binding portion is fused to the C-terminus of the same heavy chain of the first antigen-binding portion and fused to the N-terminus and C-terminus of the other heavy chain of the first antigen-binding portion.

In some embodiments, the second antigen-binding portion is fused to the C-terminus of one of the heavy chains of the first antigen-binding portion, and the third antigen-binding portion is fused to the N-terminus of the same heavy chain of the first antigen-binding portion and fused to the N-terminus of the other heavy chain of the first antigen-binding portion.

In some embodiments, the second antigen-binding portion is fused to the N-terminus of one of the heavy chains of the first antigen-binding portion, and the third antigen-binding portion is fused to the N-terminus of the same heavy chain of the first antigen-binding portion and fused to the C-terminus of the other heavy chain of the first antigen-binding portion.

In some embodiments, the second antigen-binding portion is fused to the N-terminus of one of the heavy chains of the first antigen-binding portion, and the third antigen-binding portion is fused to the N-terminus of the same heavy chain of the first antigen-binding portion and fused to the N-terminus and C-terminus of the other heavy chain of the first antigen-binding portion.

In some embodiments, the third antigen-binding portion is fused to the N-terminus of one of the light chains of the first antigen-binding portion.

In some embodiments, the third antigen-binding portion is fused to the C-terminus of one of the light chains of the first antigen-binding portion.

In some embodiments, the third antigen-binding portion is fused to the N-terminus and C-terminus of one of the light chains of the first antigen-binding portion.

In some embodiments, the third antigen-binding portion is fused to the N-terminus of both light chains of the first antigen-binding portion.

In some embodiments, the third antigen-binding portion is fused to the C-terminus of both light chains of the first antigen-binding portion.

In some embodiments, the third antigen-binding portion is fused to the N-terminus and C-terminus of both light chains of the first antigen-binding portion.

In some embodiments, the second antigen-binding portion is fused to the N-terminus of one of the heavy chains of the first antigen-binding portion, and the third antigen-binding portion is fused to the N-terminus of one of the light chains of the first antigen-binding portion.

In some embodiments, the second antigen-binding portion is fused to the N-terminus of one of the heavy chains of the first antigen-binding portion, and the third antigen-binding portion is fused to the C-terminus of one of the light chains of the first antigen-binding portion.

In some embodiments, the second antigen-binding portion is fused to the N-terminus of one of the heavy chains of the first antigen-binding portion, and the third antigen-binding portion is fused to the N-terminus and C-terminus of one of the light chains of the first antigen-binding portion.

In some embodiments, the second antigen-binding portion is fused to the C-terminus of one of the heavy chains of the first antigen-binding portion, and the third antigen-binding portion is fused to the N-terminus of one of the light chains of the first antigen-binding portion.

In some embodiments, the second antigen-binding portion is fused to the C-terminus of one of the heavy chains of the first antigen-binding portion, and the third antigen-binding portion is fused to the C-terminus of one of the light chains of the first antigen-binding portion.

In some embodiments, the second antigen-binding portion is fused to the C-terminus of one of the heavy chains of the first antigen-binding portion, and the third antigen-binding portion is fused to the N-terminus and C-terminus of one of the light chains of the first antigen-binding portion.

In some embodiments, the second antigen-binding portion is fused to the N-terminus and C-terminus of one of the heavy chains of the first antigen-binding portion, and the third antigen-binding portion is fused to the N-terminus of one of the light chains of the first antigen-binding portion.

In some embodiments, the second antigen-binding portion is fused to the N-terminus and C-terminus of one of the heavy chains of the first antigen-binding portion, and the third antigen-binding portion is fused to the C-terminus of one of the light chains of the first antigen-binding portion.

In some embodiments, the second antigen-binding portion is fused to the N-terminus and C-terminus of one of the heavy chains of the first antigen-binding portion, and the third antigen-binding portion is fused to the N-terminus and C-terminus of one of the light chains of the first antigen-binding portion.

In some embodiments, the second antigen-binding portion is fused to the N-terminus of one of the heavy chains of the first antigen-binding portion, and the third antigen-binding portion is fused to the N-terminus of both light chains of the first antigen-binding portion.

In some embodiments, the second antigen-binding portion is fused to the N-terminus of one of the heavy chains of the first antigen-binding portion, and the third antigen-binding portion is fused to the C-terminus of both light chains of the first antigen-binding portion.

In some embodiments, the second antigen-binding portion is fused to the N-terminus of one of the heavy chains of the first antigen-binding portion, and the third antigen-binding portion is fused to the N-terminus and C-terminus of both light chains of the first antigen-binding portion.

In some embodiments, the second antigen-binding portion is fused to the C-terminus of one of the heavy chains of the first antigen-binding portion, and the third antigen-binding portion is fused to the N-terminus of both light chains of the first antigen-binding portion.

In some embodiments, the second antigen-binding portion is fused to the C-terminus of one of the heavy chains of the first antigen-binding portion, and the third antigen-binding portion is fused to the C-terminus of both light chains of the first antigen-binding portion.

In some embodiments, the second antigen-binding portion is fused to the C-terminus of one of the heavy chains of the first antigen-binding portion, and the third antigen-binding portion is fused to the N-terminus and C-terminus of both light chains of the first antigen-binding portion.

In some embodiments, the second antigen-binding portion is fused to the N-terminus and C-terminus of one of the heavy chains of the first antigen-binding portion, and the third antigen-binding portion is fused to the N-terminus of both light chains of the first antigen-binding portion.

In some embodiments, the second antigen-binding portion is fused to the N-terminus and C-terminus of one of the heavy chains of the first antigen-binding portion, and the third antigen-binding portion is fused to the C-terminus of both light chains of the first antigen-binding portion.

In some embodiments, the second antigen-binding portion is fused to the N-terminus and C-terminus of one of the heavy chains of the first antigen-binding portion, and the third antigen-binding portion is fused to the N-terminus and C-terminus of both light chains of the first antigen-binding portion.

In some embodiments, the second antigen-binding portion is fused to the N-terminus of one of the light chains of the first antigen-binding portion.

In some embodiments, the second antigen-binding portion is fused to the C-terminus of one of the light chains of the first antigen-binding portion.

In some embodiments, the second antigen-binding portion is fused to the N-terminus and C-terminus of one of the light chains of the first antigen-binding portion.

In some embodiments, the second antigen-binding portion is fused to the N-terminus of one of the light chains of the first antigen-binding portion, and the third antigen-binding portion is fused to the C-terminus of the same light chain of the first antigen-binding portion.

In some embodiments, the second antigen-binding portion is fused to the C-terminus of one of the light chains of the first antigen-binding portion, and the third antigen-binding portion is fused to the N-terminus of the same light chain of the first antigen-binding portion.

In some embodiments, the third antigen-binding portion is fused to the N-terminus of the other light chain of the first antigen-binding portion.

In some embodiments, the third antigen-binding portion is fused to the C-terminus of the other light chain of the first antigen-binding portion.

In some embodiments, the third antigen-binding portion is fused to the N-terminus and C-terminus of the other light chain of the first antigen-binding portion.

In some embodiments, the second antigen-binding portion is fused to the N-terminus of one of the light chains of the first antigen-binding portion, and the third antigen-binding portion is fused to the N-terminus of the other light chain of the first antigen-binding portion.

In some embodiments, the second antigen-binding portion is fused to the N-terminus of one of the light chains of the first antigen-binding portion, and the third antigen-binding portion is fused to the C-terminus of the other light chain of the first antigen-binding portion.

In some embodiments, the second antigen-binding portion is fused to the N-terminus of one of the light chains of the first antigen-binding portion, and the third antigen-binding portion is fused to the N-terminus and C-terminus of the other light chain of the first antigen-binding portion.

In some embodiments, the second antigen-binding portion is fused to the C-terminus of one of the light chains of the first antigen-binding portion, and the third antigen-binding portion is fused to the N-terminus of the other light chain of the first antigen-binding portion.

In some embodiments, the second antigen-binding portion is fused to the C-terminus of one of the light chains of the first antigen-binding portion, and the third antigen-binding portion is fused to the C-terminus of the other light chain of the first antigen-binding portion.

In some embodiments, the second antigen-binding portion is fused to the C-terminus of one of the light chains of the first antigen-binding portion, and the third antigen-binding portion is fused to the N-terminus and C-terminus of the other light chain of the first antigen-binding portion.

In some embodiments, the second antigen-binding portion is fused to the N-terminus and C-terminus of one of the light chains of the first antigen-binding portion, and the third antigen-binding portion is fused to the N-terminus of the other light chain of the first antigen-binding portion.

In some embodiments, the second antigen-binding portion is fused to the N-terminus and C-terminus of one of the light chains of the first antigen-binding portion, and the third antigen-binding portion is fused to the C-terminus of the other light chain of the first antigen-binding portion.

In some embodiments, the second antigen-binding portion is fused to the N-terminus and C-terminus of one of the light chains of the first antigen-binding portion, and the third antigen-binding portion is fused to the N-terminus and C-terminus of the other light chain of the first antigen-binding portion.

In some embodiments, the second antigen-binding portion is fused to the N-terminus of one of the light chains of the first antigen-binding portion, and the third antigen-binding portion is fused to the C-terminus of the same light chain of the first antigen-binding portion and fused to the N-terminus of the other light chain of the first antigen-binding portion.

In some embodiments, the second antigen-binding portion is fused to the N-terminus of one of the light chains of the first antigen-binding portion, and the third antigen-binding portion is fused to the C-terminus of the same light chain of the first antigen-binding portion and fused to the C-terminus of the other light chain of the first antigen-binding portion.

In some embodiments, the second antigen-binding portion is fused to the N-terminus of one of the light chains of the first antigen-binding portion, and the third antigen-binding portion is fused to the C-terminus of the same light chain of the first antigen-binding portion and fused to the N-terminus and C-terminus of the other light chain of the first antigen-binding portion.

In some embodiments, the second antigen-binding portion is fused to the C-terminus of one of the light chains of the first antigen-binding portion, and the third antigen-binding portion is fused to the N-terminus of the same light chain of the first antigen-binding portion and fused to the N-terminus of the other light chain of the first antigen-binding portion.

In some embodiments, the second antigen-binding portion is fused to the N-terminus of one of the light chains of the first antigen-binding portion, and the third antigen-binding portion is fused to the N-terminus of the same light chain of the first antigen-binding portion and fused to the C-terminus of the other light chain of the first antigen-binding portion.

In some embodiments, the second antigen-binding portion is fused to the N-terminus of one of the light chains of the first antigen-binding portion, and the third antigen-binding portion is fused to the N-terminus of the same light chain of the first antigen-binding portion and fused to the N-terminus and C-terminus of the other light chain of the first antigen-binding portion.

In some embodiments, the third antigen-binding portion is fused to the N-terminus of one of the heavy chains of the first antigen-binding portion.

In some embodiments, the third antigen-binding portion is fused to the C-terminus of one of the heavy chains of the first antigen-binding portion.

In some embodiments, the third antigen-binding portion is fused to the N-terminus and C-terminus of one of the heavy chains of the first antigen-binding portion.

In some embodiments, the third antigen-binding portion is fused to the N-terminus of both heavy chains of the first antigen-binding portion.

In some embodiments, the third antigen-binding portion is fused to the C-terminus of both heavy chains of the first antigen-binding portion.

In some embodiments, the third antigen-binding portion is fused to the N-terminus and C-terminus of both heavy chains of the first antigen-binding portion.

In some embodiments, the second antigen-binding portion is fused to the N-terminus of one of the light chains of the first antigen-binding portion, and the third antigen-binding portion is fused to the N-terminus of one of the heavy chains of the first antigen-binding portion.

In some embodiments, the second antigen-binding portion is fused to the N-terminus of one of the light chains of the first antigen-binding portion, and the third antigen-binding portion is fused to the C-terminus of one of the heavy chains of the first antigen-binding portion.

In some embodiments, the second antigen-binding portion is fused to the N-terminus of one of the light chains of the first antigen-binding portion, and the third antigen-binding portion is fused to the N-terminus and C-terminus of one of the heavy chains of the first antigen-binding portion.

In some embodiments, the second antigen-binding portion is fused to the C-terminus of one of the light chains of the first antigen-binding portion, and the third antigen-binding portion is fused to the N-terminus of one of the heavy chains of the first antigen-binding portion.

In some embodiments, the second antigen-binding portion is fused to the C-terminus of one of the light chains of the first antigen-binding portion, and the third antigen-binding portion is fused to the C-terminus of one of the heavy chains of the first antigen-binding portion.

In some embodiments, the second antigen-binding portion is fused to the C-terminus of one of the light chains of the first antigen-binding portion, and the third antigen-binding portion is fused to the N-terminus and C-terminus of one of the heavy chains of the first antigen-binding portion.

In some embodiments, the second antigen-binding portion is fused to the N-terminus and C-terminus of one of the light chains of the first antigen-binding portion, and the third antigen-binding portion is fused to the N-terminus of one of the heavy chains of the first antigen-binding portion.

In some embodiments, the second antigen-binding portion is fused to the N-terminus and C-terminus of one of the light chains of the first antigen-binding portion, and the third antigen-binding portion is fused to the C-terminus of one of the heavy chains of the first antigen-binding portion.

In some embodiments, the second antigen-binding portion is fused to the N-terminus and C-terminus of one of the light chains of the first antigen-binding portion, and the third antigen-binding portion is fused to the N-terminus and C-terminus of one of the heavy chains of the first antigen-binding portion.

In some embodiments, the second antigen-binding portion is fused to the N-terminus of one of the light chains of the first antigen-binding portion, and the third antigen-binding portion is fused to the N-terminus of both heavy chains of the first antigen-binding portion.

In some embodiments, the second antigen-binding portion is fused to the N-terminus of one of the light chains of the first antigen-binding portion, and the third antigen-binding portion is fused to the C-terminus of both heavy chains of the first antigen-binding portion.

In some embodiments, the second antigen-binding portion is fused to the N-terminus of one of the light chains of the first antigen-binding portion, and the third antigen-binding portion is fused to the N-terminus and C-terminus of both heavy chains of the first antigen-binding portion.

In some embodiments, the second antigen-binding portion is fused to the C-terminus of one of the light chains of the first antigen-binding portion, and the third antigen-binding portion is fused to the N-terminus of both heavy chains of the first antigen-binding portion.

In some embodiments, the second antigen-binding portion is fused to the C-terminus of one of the light chains of the first antigen-binding portion, and the third antigen-binding portion is fused to the C-terminus of both heavy chains of the first antigen-binding portion.

In some embodiments, the second antigen-binding portion is fused to the C-terminus of one of the light chains of the first antigen-binding portion, and the third antigen-binding portion is fused to the N-terminus and C-terminus of both heavy chains of the first antigen-binding portion.

In some embodiments, the second antigen-binding portion is fused to the N-terminus and C-terminus of one of the light chains of the first antigen-binding portion, and the third antigen-binding portion is fused to the N-terminus of both heavy chains of the first antigen-binding portion.

In some embodiments, the second antigen-binding portion is fused to the N-terminus and C-terminus of one of the light chains of the first antigen-binding portion, and the third antigen-binding portion is fused to the C-terminus of both heavy chains of the first antigen-binding portion.

In some embodiments, the second antigen-binding portion is fused to the N-terminus and C-terminus of one of the light chains of the first antigen-binding portion, and the third antigen-binding portion is fused to the N-terminus and C-terminus of both heavy chains of the first antigen-binding portion.

In some embodiments, the second antigen-binding portion is fused to the N-terminus of both heavy chains of the first antigen-binding portion.

In some embodiments, the second antigen-binding portion is fused to the C-terminus of both heavy chains of the first antigen-binding portion.

In some embodiments, the second antigen-binding portion is fused to the N-terminus and C-terminus of both heavy chains of the first antigen-binding portion.

In some embodiments, the second antigen-binding portion is fused to the N-terminus of both heavy chains of the first antigen-binding portion, and the third antigen-binding portion is fused to the C-terminus of both heavy chains of the first antigen-binding portion.

In some embodiments, the second antigen-binding portion is fused to the C-terminus of both heavy chains of the first antigen-binding portion, and the third antigen-binding portion is fused to the N-terminus of both heavy chains of the first antigen-binding portion.

In some embodiments, the third antigen-binding portion is fused to the N-terminus of one of the light chains of the first antigen-binding portion.

In some embodiments, the third antigen-binding portion is fused to the C-terminus of one of the light chains of the first antigen-binding portion.

In some embodiments, the third antigen-binding portion is fused to the N-terminus and C-terminus of one of the light chains of the first antigen-binding portion.

In some embodiments, the third antigen-binding portion is fused to the N-terminus of both light chains of the first antigen-binding portion.

In some embodiments, the third antigen-binding portion is fused to the C-terminus of both light chains of the first antigen-binding portion.

In some embodiments, the third antigen-binding portion is fused to the N-terminus and C-terminus of both light chains of the first antigen-binding portion.

In some embodiments, the second antigen-binding portion is fused to the N-terminus of both heavy chains of the first antigen-binding portion, and the third antigen-binding portion is fused to the N-terminus of one of the light chains of the first antigen-binding portion.

In some embodiments, the second antigen-binding portion is fused to the N-terminus of both heavy chains of the first antigen-binding portion, and the third antigen-binding portion is fused to the C-terminus of one of the light chains of the first antigen-binding portion.

In some embodiments, the second antigen-binding portion is fused to the N-terminus of both heavy chains of the first antigen-binding portion, and the third antigen-binding portion is fused to the N-terminus and C-terminus of one of the light chains of the first antigen-binding portion.

In some embodiments, the second antigen-binding portion is fused to the N-terminus of both heavy chains of the first antigen-binding portion, and the third antigen-binding portion is fused to the N-terminus of both light chains of the first antigen-binding portion.

In some embodiments, the second antigen-binding portion is fused to the N-terminus of both heavy chains of the first antigen-binding portion, and the third antigen-binding portion is fused to the C-terminus of both light chains of the first antigen-binding portion.

In some embodiments, the second antigen-binding portion is fused to the N-terminus of both heavy chains of the first antigen-binding portion, and the third antigen-binding portion is fused to the N-terminus and C-terminus of both light chains of the first antigen-binding portion.

In some embodiments, the second antigen-binding portion is fused to the C-terminus of both heavy chains of the first antigen-binding portion, and the third antigen-binding portion is fused to the N-terminus of one of the light chains of the first antigen-binding portion.

In some embodiments, the second antigen-binding portion is fused to the C-terminus of both heavy chains of the first antigen-binding portion, and the third antigen-binding portion is fused to the C-terminus of one of the light chains of the first antigen-binding portion.

In some embodiments, the second antigen-binding portion is fused to the C-terminus of both heavy chains of the first antigen-binding portion, and the third antigen-binding portion is fused to the N-terminus and C-terminus of one of the light chains of the first antigen-binding portion.

In some embodiments, the second antigen-binding portion is fused to the C-terminus of both heavy chains of the first antigen-binding portion, and the third antigen-binding portion is fused to the N-terminus of both light chains of the first antigen-binding portion.

In some embodiments, the second antigen-binding portion is fused to the C-terminus of both heavy chains of the first antigen-binding portion, and the third antigen-binding portion is fused to the C-terminus of both light chains of the first antigen-binding portion.

In some embodiments, the second antigen-binding portion is fused to the C-terminus of both heavy chains of the first antigen-binding portion, and the third antigen-binding portion is fused to the N-terminus and C-terminus of both light chains of the first antigen-binding portion.

In some embodiments, the second antigen-binding portion is fused to the N-terminus and C-terminus of both heavy chains of the first antigen-binding portion, and the third antigen-binding portion is fused to the N-terminus of one of the light chains of the first antigen-binding portion.

In some embodiments, the second antigen-binding portion is fused to the N-terminus and C-terminus of both heavy chains of the first antigen-binding portion, and the third antigen-binding portion is fused to the C-terminus of one of the light chains of the first antigen-binding portion.

In some embodiments, the second antigen-binding portion is fused to the N-terminus and C-terminus of both heavy chains of the first antigen-binding portion, and the third antigen-binding portion is fused to the N-terminus and C-terminus of one of the light chains of the first antigen-binding portion.

In some embodiments, the second antigen-binding portion is fused to the N-terminus and C-terminus of both heavy chains of the first antigen-binding portion, and the third antigen-binding portion is fused to the N-terminus of both light chains of the first antigen-binding portion.

In some embodiments, the second antigen-binding portion is fused to the N-terminus and C-terminus of both heavy chains of the first antigen-binding portion, and the third antigen-binding portion is fused to the C-terminus of both light chains of the first antigen-binding portion.

In some embodiments, the second antigen-binding portion is fused to the N-terminus and C-terminus of both heavy chains of the first antigen-binding portion, and the third antigen-binding portion is fused to the N-terminus and C-terminus of both light chains of the first antigen-binding portion.

In some embodiments, the second antigen-binding portion is fused to the N-terminus of both light chains of the first antigen-binding portion.

In some embodiments, the second antigen-binding portion is fused to the C-terminus of both light chains of the first antigen-binding portion.

In some embodiments, the second antigen-binding portion is fused to the N-terminus and C-terminus of both light chains of the first antigen-binding portion.

In some embodiments, the second antigen-binding portion is fused to the N-terminus of both light chains of the first antigen-binding portion, and the third antigen-binding portion is fused to the C-terminus of both light chains of the first antigen-binding portion.

In some embodiments, the second antigen-binding portion is fused to the C-terminus of both light chains of the first antigen-binding portion, and the third antigen-binding portion is fused to the N-terminus of both light chains of the first antigen-binding portion.

In some embodiments, the third antigen-binding portion is fused to the N-terminus of one of the heavy chains of the first antigen-binding portion.

In some embodiments, the third antigen-binding portion is fused to the C-terminus of one of the heavy chains of the first antigen-binding portion.

In some embodiments, the third antigen-binding portion is fused to the N-terminus and C-terminus of one of the heavy chains of the first antigen-binding portion.

In some embodiments, the third antigen-binding portion is fused to the N-terminus of both heavy chains of the first antigen-binding portion.

In some embodiments, the third antigen-binding portion is fused to the C-terminus of both heavy chains of the first antigen-binding portion.

In some embodiments, the third antigen-binding portion is fused to the N-terminus and C-terminus of both heavy chains of the first antigen-binding portion.

In some embodiments, the second antigen-binding portion is fused to the N-terminus of both light chains of the first antigen-binding portion, and the third antigen-binding portion is fused to the N-terminus of one of the heavy chains of the first antigen-binding portion.

In some embodiments, the second antigen-binding portion is fused to the N-terminus of both light chains of the first antigen-binding portion, and the third antigen-binding portion is fused to the C-terminus of one of the heavy chains of the first antigen-binding portion.

In some embodiments, the second antigen-binding portion is fused to the N-terminus of both light chains of the first antigen-binding portion, and the third antigen-binding portion is fused to the N-terminus and C-terminus of one of the heavy chains of the first antigen-binding portion.

In some embodiments, the second antigen-binding portion is fused to the N-terminus of both light chains of the first antigen-binding portion, and the third antigen-binding portion is fused to the N-terminus of both heavy chains of the first antigen-binding portion.

In some embodiments, the second antigen-binding portion is fused to the N-terminus of both light chains of the first antigen-binding portion, and the third antigen-binding portion is fused to the C-terminus of both heavy chains of the first antigen-binding portion.

In some embodiments, the second antigen-binding portion is fused to the N-terminus of both light chains of the first antigen-binding portion, and the third antigen-binding portion is fused to the N-terminus and C-terminus of both heavy chains of the first antigen-binding portion.

In some embodiments, the second antigen-binding portion is fused to the C-terminus of both light chains of the first antigen-binding portion, and the third antigen-binding portion is fused to the N-terminus of one of the heavy chains of the first antigen-binding portion.

In some embodiments, the second antigen-binding portion is fused to the C-terminus of both light chains of the first antigen-binding portion, and the third antigen-binding portion is fused to the C-terminus of one of the heavy chains of the first antigen-binding portion.

In some embodiments, the second antigen-binding portion is fused to the C-terminus of both light chains of the first antigen-binding portion, and the third antigen-binding portion is fused to the N-terminus and C-terminus of one of the heavy chains of the first antigen-binding portion.

In some embodiments, the second antigen-binding portion is fused to the C-terminus of both light chains of the first antigen-binding portion, and the third antigen-binding portion is fused to the N-terminus of both heavy chains of the first antigen-binding portion.

In some embodiments, the second antigen-binding portion is fused to the C-terminus of both light chains of the first antigen-binding portion, and the third antigen-binding portion is fused to the C-terminus of both heavy chains of the first antigen-binding portion.

In some embodiments, the second antigen-binding portion is fused to the C-terminus of both light chains of the first antigen-binding portion, and the third antigen-binding portion is fused to the N-terminus and C-terminus of both heavy chains of the first antigen-binding portion.

In some embodiments, the second antigen-binding portion is fused to the N-terminus and C-terminus of both light chains of the first antigen-binding portion, and the third antigen-binding portion is fused to the N-terminus of one of the heavy chains of the first antigen-binding portion.

In some embodiments, the second antigen-binding portion is fused to the N-terminus and C-terminus of both light chains of the first antigen-binding portion, and the third antigen-binding portion is fused to the C-terminus of one of the heavy chains of the first antigen-binding portion.

In some embodiments, the second antigen-binding portion is fused to the N-terminus and C-terminus of both light chains of the first antigen-binding portion, and the third antigen-binding portion is fused to the N-terminus and C-terminus of one of the heavy chains of the first antigen-binding portion.

In some embodiments, the second antigen-binding portion is fused to the N-terminus and C-terminus of both light chains of the first antigen-binding portion, and the third antigen-binding portion is fused to the N-terminus of both heavy chains of the first antigen-binding portion.

In some embodiments, the second antigen-binding portion is fused to the N-terminus and C-terminus of both light chains of the first antigen-binding portion, and the third antigen-binding portion is fused to the C-terminus of both heavy chains of the first antigen-binding portion.

In some embodiments, the second antigen-binding portion is fused to the N-terminus and C-terminus of both light chains of the first antigen-binding portion, and the third antigen-binding portion is fused to the N-terminus and C-terminus of both heavy chains of the first antigen-binding portion.

In some embodiments, the third antigen-binding portion replaces one or more binding units of: Fab region or Fv region or VH, CH1 domain, CH2 domain, or CH3 domain of the first antigen-binding portion, and/or the second antigen-binding portion.

In some embodiments, the third antigen-binding portion replaces the Fab region of the first antigen-binding portion and/or the second antigen-binding portion.

In some embodiments, the third antigen-binding portion replaces the Fv region of the first antigen-binding portion and/or the second antigen-binding portion.

In some embodiments, the third antigen-binding portion replaces the VH portion of the first antigen-binding portion and/or the second antigen-binding portion.

In some embodiments, the third antigen-binding portion replaces the CH1 domain of the first antigen-binding portion and/or the second antigen-binding portion.

In some embodiments, the third antigen-binding portion replaces the CH2 domain of the first antigen-binding portion and/or the second antigen-binding portion.

In some embodiments, the third antigen-binding portion replaces the CH3 domain of the first antigen-binding portion and/or the second antigen-binding portion.

In some embodiments, the third antigen-binding portion is located between any one of: VH domain, CH1 domain, CH2 domain, and CH3 domain of the first antigen-binding portion and/or the second antigen-binding portion.

In some embodiments, the third antigen-binding portion is located between the VH domain and the CH1 domain of the first antigen-binding portion and/or the second antigen-binding portion.

In some embodiments, the third antigen-binding portion is located between the CH1 domain and the CH2 domain of the first antigen-binding portion and/or the second antigen-binding portion.

In some embodiments, the third antigen-binding portion is located between the CH2 domain and the CH3 domain of the first antigen-binding portion and/or the second antigen-binding portion.

In some embodiments, the second antigen-binding portion is fused to at least one light chain of the first antigen-binding portion.

In some embodiments, the second antigen-binding portion is fused to one of the light chains of the first antigen-binding portion.

In some embodiments, the second antigen-binding portion is fused to the N-terminus of one of the light chains of the first antigen-binding portion.

In some embodiments, the second antigen-binding portion is fused to the C-terminus of one of the light chains of the first antigen-binding portion.

In some embodiments, the second antigen-binding portion is fused to the N-terminus and C-terminus of one of the light chains of the first antigen-binding portion.

In some embodiments, the second antigen-binding portion is fused to both light chains of the first antigen-binding portion.

In some embodiments, the second antigen-binding portion is fused to the N-terminus of both light chains of the first antigen-binding portion.

In some embodiments, the second antigen-binding portion is fused to the C-terminus of both light chains of the first antigen-binding portion.

In some embodiments, the second antigen-binding portion is fused to the N-terminus and C-terminus of both light chains of the first antigen-binding portion.

In some embodiments, the second antigen-binding portion is fused to the N-terminus of one of the light chains of the first antigen-binding portion, and the third antigen-binding portion replaces the Fab region of the first antigen-binding portion and/or the second antigen-binding portion.

In some embodiments, the second antigen-binding portion is fused to the C-terminus of one of the light chains of the first antigen-binding portion, and the third antigen-antigen-binding portion.

In some embodiments, the second antigen-binding portion is fused to the N-terminus and C-terminus of one of the light chains of the first antigen-binding portion, and the third antigen-binding portion replaces the Fab region of the first antigen-binding portion and/or the second antigen-binding portion.

In some embodiments, the second antigen-binding portion is fused to the N-terminus of both light chains of the first antigen-binding portion, and the third antigen-binding portion replaces the Fab region of the first antigen-binding portion and/or the second antigen-binding portion.

In some embodiments, the second antigen-binding portion is fused to the C-terminus of both light chains of the first antigen-binding portion, and the third antigen-binding portion replaces the Fab region of the first antigen-binding portion and/or the second antigen-binding portion.

In some embodiments, the second antigen-binding portion is fused to the N-terminus and C-terminus of both light chains of the first antigen-binding portion, and the third antigen-binding portion replaces the Fab region of the first antigen-binding portion and/or the second antigen-binding portion.

In some embodiments, the second antigen-binding portion is fused to the N-terminus of one of the light chains of the first antigen-binding portion, and the third antigen-binding portion replaces the Fv region of the first antigen-binding portion and/or the second antigen-binding portion.

In some embodiments, the second antigen-binding portion is fused to the C-terminus of one of the light chains of the first antigen-binding portion, and the third antigen-binding portion replaces the Fv region of the first antigen-binding portion and/or the second antigen-binding portion.

In some embodiments, the second antigen-binding portion is fused to the N-terminus and C-terminus of one of the light chains of the first antigen-binding portion, and the third antigen-binding portion replaces the Fv region of the first antigen-binding portion and/or the second antigen-binding portion.

In some embodiments, the second antigen-binding portion is fused to the N-terminus of both light chains of the first antigen-binding portion, and the third antigen-binding portion replaces the Fv region of the first antigen-binding portion and/or the second antigen-binding portion.

In some embodiments, the second antigen-binding portion is fused to the C-terminus of both light chains of the first antigen-binding portion, and the third antigen-binding portion replaces the Fv region of the first antigen-binding portion and/or the second antigen-binding portion.

In some embodiments, the second antigen-binding portion is fused to the N-terminus and C-terminus of both light chains of the first antigen-binding portion, and the third antigen-binding portion replaces the Fv region of the first antigen-binding portion and/or the second antigen-binding portion.

In some embodiments, the second antigen-binding portion is fused to at least one heavy chain of the first antigen-binding portion.

In some embodiments, the second antigen-binding portion is fused to one of the heavy chains of the first antigen-binding portion.

In some embodiments, the second antigen-binding portion is fused to the N-terminus of one of the heavy chains of the first antigen-binding portion.

In some embodiments, the second antigen-binding portion is fused to the C-terminus of one of the heavy chains of the first antigen-binding portion.

In some embodiments, the second antigen-binding portion is fused to the N-terminus and C-terminus of one of the heavy chains of the first antigen-binding portion.

In some embodiments, the second antigen-binding portion is fused to both heavy chains of the first antigen-binding portion.

In some embodiments, the second antigen-binding portion is fused to the N-terminus of both heavy chains of the first antigen-binding portion.

In some embodiments, the second antigen-binding portion is fused to the C-terminus of both heavy chains of the first antigen-binding portion.

In some embodiments, the second antigen-binding portion is fused to the N-terminus and C-terminus of both heavy chains of the first antigen-binding portion.

In some embodiments, the second antigen-binding portion is fused to the N-terminus of one of the heavy chains of the first antigen-binding portion, and the third antigen-binding portion replaces the Fab region of the first antigen-binding portion and/or the second antigen-binding portion.

In some embodiments, the second antigen-binding portion is fused to the C-terminus of one of the heavy chains of the first antigen-binding portion, and the third antigen-antigen-binding portion.

In some embodiments, the second antigen-binding portion is fused to the N-terminus and C-terminus of one of the heavy chains of the first antigen-binding portion, and the third antigen-binding portion replaces the Fab region of the first antigen-binding portion and/or the second antigen-binding portion.

In some embodiments, the second antigen-binding portion is fused to the N-terminus of both heavy chains of the first antigen-binding portion, and the third antigen-binding portion replaces the Fab region of the first antigen-binding portion and/or the second antigen-binding portion.

In some embodiments, the second antigen-binding portion is fused to the C-terminus of both heavy chains of the first antigen-binding portion, and the third antigen-binding portion replaces the Fab region of the first antigen-binding portion and/or the second antigen-binding portion.

In some embodiments, the second antigen-binding portion is fused to the N-terminus and C-terminus of both heavy chains of the first antigen-binding portion, and the third antigen-binding portion replaces the Fab region of the first antigen-binding portion and/or the second antigen-binding portion.

In some embodiments, the second antigen-binding portion is fused to the N-terminus of one of the heavy chains of the first antigen-binding portion, and the third antigen-binding portion replaces the Fv region of the first antigen-binding portion and/or the second antigen-binding portion.

In some embodiments, the second antigen-binding portion is fused to the C-terminus of one of the heavy chains of the first antigen-binding portion, and the third antigen-binding portion replaces the Fv region of the first antigen-binding portion and/or the second antigen-binding portion.

In some embodiments, the second antigen-binding portion is fused to the N-terminus and C-terminus of one of the heavy chains of the first antigen-binding portion, and the third antigen-binding portion replaces the Fv region of the first antigen-binding portion and/or the second antigen-binding portion.

In some embodiments, the second antigen-binding portion is fused to the N-terminus of both heavy chains of the first antigen-binding portion, and the third antigen-binding portion replaces the Fv region of the first antigen-binding portion and/or the second antigen-binding portion.

In some embodiments, the second antigen-binding portion is fused to the C-terminus of both heavy chains of the first antigen-binding portion, and the third antigen-binding portion replaces the Fv region of the first antigen-binding portion and/or the second antigen-binding portion.

In some embodiments, the second antigen-binding portion is fused to the N-terminus and C-terminus of both heavy chains of the first antigen-binding portion, and the third antigen-binding portion replaces the Fv region of the first antigen-binding portion and/or the second antigen-binding portion.

In some embodiments, the multispecific antigen-binding protein comprises a first Fc region and a second Fc region.

In some embodiments, the first Fc region and the second Fc region are the same Fc or different Fc.

In some embodiments, the first Fc region is knob-Fc, and the second Fc region is hole-Fc.

In some embodiments, the first Fc region is hole-Fc, and the second Fc region is knob-Fc.

In some embodiments, VH and VL of the first antigen-binding portion and/or the second antigen-binding portion and/or the third antigen-binding portion are interchanged.

In some embodiments, CL and CH1 of the first antigen-binding portion and/or the second antigen-binding portion and/or the third antigen-binding portion are interchanged.

In some embodiments, CH3 of the first Fc region is replaced by CL or CH1, and CH3 of the second Fc region is replaced by CL or CH1.

In some embodiments, VH and VL of the first antigen-binding portion and/or the second antigen-binding portion and/or the third antigen-binding portion are interchanged, CL and CH1 of the first antigen-binding portion and/or the second antigen-binding portion and/or the third antigen-binding portion are interchanged.

In some embodiments, VH and VL of the first antigen-binding portion and/or the second antigen-binding portion and/or the third antigen-binding portion are interchanged, CH3 of the first Fc region is replaced by CL or CH1, and CH3 of the second Fc region is replaced by CL or CH1.

In some embodiments, CL and CH1 of the first antigen-binding portion and/or the second antigen-binding portion and/or the third antigen-binding portion are interchanged, CH3 of the first Fc region is replaced by CL or CH1, and CH3 of the second Fc region is replaced by CL or CH1.

In some embodiments, VH and VL of the first antigen-binding portion and/or the second antigen-binding portion and/or the third antigen-binding portion are interchanged, CL and CH1 of the first antigen-binding portion and/or the second antigen-binding portion and/or the third antigen-binding portion are interchanged, CH3 of the first Fc region is replaced by CL or CH1, and CH3 of the second Fc region is replaced by CL or CH1.

In some embodiments, the heavy chain and/or the Fc fragment of the first antigen-binding portion and/or the second antigen-binding portion and/or the third antigen-binding portion comprises one or more amino acid substitutions, and the substitutions form an ionic bond between the heavy chain and the Fc fragment.

In some embodiments, the second antigen-binding portion is fused to the N-terminus of both light chains of the first antigen-binding portion, and the third antigen-binding portion is fused to the C-terminus of both heavy chains of the first antigen-binding portion.

In some embodiments, the second antigen-binding portion is fused to the N-terminus of both heavy chains of the first antigen-binding portion, and the third antigen-binding portion is fused to the C-terminus of both heavy chains of the first antigen-binding portion.

In some embodiments, the second antigen-binding portion is fused to the C-terminus of both light chains of the first antigen-binding portion, and the third antigen-binding portion is fused to the C-terminus of both heavy chains of the first antigen-binding portion.

In some embodiments, the second antigen-binding portion is fused to the N-terminus of one of the light chains of the first antigen-binding portion, and the third antigen-binding portion is fused to the C-terminus of both heavy chains of the first antigen-binding portion.

In some embodiments, the second antigen-binding portion is fused to the N-terminus of one of the light chains of the first antigen-binding portion, the VH and VL of the Fab region of the first antigen-binding portion fused to the second antigen-binding portion are exchanged, the first Fc region of the multispecific antigen-binding protein is knob-Fc, the second Fc region is hole-Fc, and the third antigen-binding portion is fused to the C-terminus of both heavy chains of the first antigen-binding portion.

In some embodiments, the second antigen-binding portion is fused to the N-terminus of one of the heavy chains of the first antigen-binding portion, the first Fc region of the multispecific antigen-binding protein is knob-Fc, the second Fc region is hole-Fc, and the third antigen-binding portion is fused to the C-terminus of both heavy chains of the first antigen-binding portion.

In some embodiments, the second antigen-binding portion is fused to the C-terminus of one of the light chains of the first antigen-binding portion, and the third antigen-binding portion is fused to the C-terminus of both heavy chains of the first antigen-binding portion.

In some embodiments, the second antigen-binding portion is fused to the C-terminus of one of the light chains of the first antigen-binding portion, the VH and VL of the Fab region of the first antigen-binding portion fused to the second antigen-binding portion are exchanged, the first Fc region of the multispecific antigen-binding protein is knob-Fc, the second Fc region is hole-Fc, and the third antigen-binding portion is fused to the C-terminus of both heavy chains of the first antigen-binding portion.

In some embodiments, the second antigen-binding portion is fused to the C-terminus of both heavy chains of the first antigen-binding portion, and the third antigen-binding portion is fused to the N-terminus of both heavy chains of the first antigen-binding portion.

In some embodiments, the second antigen-binding portion is fused to the C-terminus of one of the heavy chains of the first antigen-binding portion, the first Fc region of the multispecific antigen-binding protein is knob-Fc, the second Fc region is hole-Fc, and the third antigen-binding portion is fused to the N-terminus of both heavy chains of the first antigen-binding portion.

In some embodiments, the second antigen-binding portion replaces the Fab region of the first antigen-binding portion, the third antigen-binding portion is fused to the N-terminus of both heavy chains of the first antigen-binding portion, the first Fc region of the multispecific antigen-binding protein is knob-Fc, the second Fc region is hole-Fc.

In some embodiments, the second antigen-binding portion replaces the Fab region of the first antigen-binding portion, the third antigen-binding portion is fused to the C-terminus of both heavy chains of the first antigen-binding portion, the first Fc region of the multispecific antigen-binding protein is knob-Fc, the second Fc region is hole-Fc.

In some embodiments, the second antigen-binding portion replaces the Fv region of the first antigen-binding portion, the third antigen-binding portion is fused to the N-terminus of both heavy chains of the first antigen-binding portion, the first Fc region of the multispecific antigen-binding protein is knob-Fc, the second Fc region is hole-Fc.

In some embodiments, the second antigen-binding portion replaces the Fv region of the first antigen-binding portion, the VH and VL of the replacement portion are exchanged, and the third antigen-binding portion is fused to the N-terminus of both heavy chains of the first antigen-binding portion, the first Fc region of the multispecific antigen-binding protein is knob-Fc, the second Fc region is hole-Fc.

In some embodiments, the second antigen-binding portion replaces the Fv region of the first antigen-binding portion, the CH1 and CL of the replacement portion are exchanged, and the third antigen-binding portion is fused to the N-terminus of both heavy chains of the first antigen-binding portion, the first Fc region of the multispecific antigen-binding protein is knob-Fc, the second Fc region is hole-Fc.

In some embodiments, the second antigen-binding portion replaces the Fv region of the first antigen-binding portion, the third antigen-binding portion is fused to the C-terminus of both heavy chains of the first antigen-binding portion, the first Fc region of the multispecific antigen-binding protein is knob-Fc, the second Fc region is hole-Fc.

In some embodiments, the second antigen-binding portion replaces the Fv region of the first antigen-binding portion, the VH and VL of the replacement portion are exchanged, and the third antigen-binding portion is fused to the C-terminus of both heavy chains of the first antigen-binding portion, the first Fc region of the multispecific antigen-binding protein is knob-Fc, the second Fc region is hole-Fc.

In some embodiments, the second antigen-binding portion replaces the Fv region of the first antigen-binding portion, the CH1 and CL of the replacement portion are exchanged, and the third antigen-binding portion is fused to the C-terminus of both heavy chains of the first antigen-binding portion, the first Fc region of the multispecific antigen-binding protein is knob-Fc, the second Fc region is hole-Fc.

In some embodiments, the second antigen-binding portion is fused to the N-terminus of both heavy chains of the first antigen-binding portion, the third antigen-binding portion replaces the Fab region of the first antigen-binding portion, and the first Fc region of the multispecific antigen-binding protein is knob-Fc, the second Fc region is hole-Fc.

In some embodiments, the second antigen-binding portion is fused to the N-terminus of one of the heavy chains of the first antigen-binding portion, the third antigen-binding portion replaces the Fab region on the other side of the first antigen-binding portion, and the first Fc region of the multispecific antigen-binding protein is knob-Fc, the second Fc region is hole-Fc.

In some embodiments, the second antigen-binding portion is fused to the N-terminus of one of the heavy chains of the first antigen-binding portion, the third antigen-binding portion replaces the Fab region on the same side of the first antigen-binding portion, and the first Fc region of the multispecific antigen-binding protein is knob-Fc, the second Fc region is hole-Fc.

In some embodiments, the second antigen-binding portion is fused to the C-terminus of both heavy chains of the first antigen-binding portion, the third antigen-binding portion replaces the Fab region of the first antigen-binding portion, and the first Fc region of the multispecific antigen-binding protein is knob-Fc, the second Fc region is hole-Fc.

In some embodiments, the second antigen-binding portion is fused to the C-terminus of one of the heavy chains of the first antigen-binding portion, the third antigen-binding portion replaces the Fab region on the other side of the first antigen-binding portion, and the first Fc region of the multispecific antigen-binding protein is knob-Fc, the second Fc region is hole-Fc.

In some embodiments, the second antigen-binding portion is fused to the C-terminus of one of the heavy chains of the first antigen-binding portion, the third antigen-binding portion replaces the Fab region on the same side of the first antigen-binding portion, and the first Fc region of the multispecific antigen-binding protein is knob-Fc, the second Fc region is hole-Fc.

In some embodiments, the second antigen-binding portion is fused to the N-terminus of both heavy chains of the first antigen-binding portion, the third antigen-binding portion replaces the Fab region of the first antigen-binding portion, and the first Fc region of the multispecific antigen-binding protein is knob-Fc, the second Fc region is hole-Fc.

In some embodiments, the second antigen-binding portion is fused to the N-terminus of one of the heavy chains of the first antigen-binding portion, the third antigen-binding portion replaces the Fab region of the first antigen-binding portion, and the first Fc region of the multispecific antigen-binding protein is knob-Fc, the second Fc region is hole-Fc.

In some embodiments, the second antigen-binding portion is fused to the N-terminus of one of the heavy chains of the first antigen-binding portion, the third antigen-binding portion replaces the Fab region on the side where the first antigen-binding portion is fused to the second antigen-binding portion, and the first Fc region of the multispecific antigen-binding protein is knob-Fc, the second Fc region is hole-Fc.

In some embodiments, the second antigen-binding portion is fused to the C-terminus of both heavy chains of the first antigen-binding portion, the third antigen-binding portion replaces the Fab region of the first antigen-binding portion, and the first Fc region of the multispecific antigen-binding protein is knob-Fc, the second Fc region is hole-Fc.

In some embodiments, the second antigen-binding portion is fused to the C-terminus of one of the heavy chains of the first antigen-binding portion, the third antigen-binding portion replaces the Fab region of the first antigen-binding portion, and the first Fc region of the multispecific antigen-binding protein is knob-Fc, the second Fc region is hole-Fc.

In some embodiments, the second antigen-binding portion is fused to the C-terminus of one of the heavy chains of the first antigen-binding portion, the third antigen-binding portion replaces the Fab region on the side where the first antigen-binding portion is fused to the second antigen-binding portion, and the first Fc region of the multispecific antigen-binding protein is knob-Fc, the second Fc region is hole-Fc.

In some embodiments, the second antigen-binding portion is fused to the N-terminus of both heavy chains of the first antigen-binding portion, the third antigen-binding portion replaces the Fv region on the side of the first antigen-binding portion, and the first Fc region of the multispecific antigen-binding protein is knob-Fc, the second Fc region is hole-Fc.

In some embodiments, the second antigen-binding portion is fused to the N-terminus of one of the heavy chains of the first antigen-binding portion, the third antigen-binding portion replaces the Fv region on the other side of the first antigen-binding portion, and the first Fc region of the multispecific antigen-binding protein is knob-Fc, the second Fc region is hole-Fc.

In some embodiments, the second antigen-binding portion is fused to the N-terminus of one of the heavy chains of the first antigen-binding portion, the third antigen-binding portion replaces the Fv region on the same side of the first antigen-binding portion, and the first Fc region of the multispecific antigen-binding protein is knob-Fc, the second Fc region is hole-Fc.

In some embodiments, the second antigen-binding portion is fused to the C-terminus of both heavy chains of the first antigen-binding portion, the third antigen-binding portion replaces the Fv region on the side of the first antigen-binding portion, and the first Fc region of the multispecific antigen-binding protein is knob-Fc, the second Fc region is hole-Fc.

In some embodiments, the second antigen-binding portion is fused to the C-terminus of one of the heavy chains of the first antigen-binding portion, the third antigen-binding portion replaces the Fv region on the other side of the first antigen-binding portion, and the first Fc region of the multispecific antigen-binding protein is knob-Fc, the second Fc region is hole-Fc.

In some embodiments, the second antigen-binding portion is fused to the C-terminus of one of the heavy chains of the first antigen-binding portion, the third antigen-binding portion replaces the Fv region on the same side of the first antigen-binding portion, and the first Fc region of the multispecific antigen-binding protein is knob-Fc, the second Fc region is hole-Fc.

In some embodiments, the first antigen-binding portion is a full-length antibody, the second antigen-binding portion and the third antigen-binding portion are both single-domain antibodies (VHH), the second antigen-binding portion is fused to the N-terminus of both light chains of the first antigen-binding portion, and the third antigen-binding portion is fused to the C-terminus of both heavy chains of the first antigen-binding portion.

In some embodiments, the first antigen-binding portion is a full-length antibody, the second antigen-binding portion and the third antigen-binding portion are both single-domain antibodies (VHH), the second antigen-binding portion is fused to the N-terminus of both heavy chains of the first antigen-binding portion, and the third antigen-binding portion is fused to the C-terminus of both heavy chains of the first antigen-binding portion.

In some embodiments, the first antigen-binding portion is a full-length antibody, the second antigen-binding portion and the third antigen-binding portion are both single-domain antibodies (VHH), the second antigen-binding portion is fused to the C-terminus of both light chains of the first antigen-binding portion, and the third antigen-binding portion is fused to the C-terminus of both heavy chains of the first antigen-binding portion.

In some embodiments, the first antigen-binding portion is a full-length antibody, the second antigen-binding portion and the third antigen-binding portion are both single-domain antibodies (VHH), the second antigen-binding portion is fused to the N-terminus of one of the light chains of the first antigen-binding portion, the VH and VL of the Fab region of the first antigen-binding portion fused to the second antigen-binding portion are exchanged, and the third antigen-binding portion is fused to the C-terminus of both heavy chains of the first antigen-binding portion, the first Fc region of the multispecific antigen-binding protein is knob-Fc, the second Fc region is hole-Fc.

In some embodiments, the first antigen-binding portion is a full-length antibody, the second antigen-binding portion and the third antigen-binding portion are both single-domain antibodies (VHH), the second antigen-binding portion is fused to the N-terminus of one of the heavy chains of the first antigen-binding portion, and the third antigen-binding portion is fused to the C-terminus of both heavy chains of the first antigen-binding portion, the first Fc region of the multispecific antigen-binding protein is knob-Fc, the second Fc region is hole-Fc.

In some embodiments, the first antigen-binding portion is a full-length antibody, the second antigen-binding portion and the third antigen-binding portion are both single-domain antibodies (VHH), the second antigen-binding portion is fused to the C-terminus of one of the light chains of the first antigen-binding portion, the VH and VL of the Fab region of the first antigen-binding portion fused to the second antigen-binding portion are exchanged, and the third antigen-binding portion is fused to the C-terminus of both heavy chains of the first antigen-binding portion, the first Fc region of the multispecific antigen-binding protein is knob-Fc, the second Fc region is hole-Fc.

In some embodiments, the first antigen-binding portion is a full-length antibody, the second antigen-binding portion and the third antigen-binding portion are both single-domain antibodies (VHH), the second antigen-binding portion is fused to the C-terminus of both heavy chains of the first antigen-binding portion, and the third antigen-binding portion is fused to the N-terminus of both heavy chains of the first antigen-binding portion.

In some embodiments, the first antigen-binding portion is a full-length antibody, the second antigen-binding portion and the third antigen-binding portion are both single-domain antibodies (VHH), the second antigen-binding portion is fused to the C-terminus of one of the heavy chains of the first antigen-binding portion, and the third antigen-binding portion is fused to the N-terminus of both heavy chains of the first antigen-binding portion, the first Fc region of the multispecific antigen-binding protein is knob-Fc, the second Fc region is hole-Fc.

In some embodiments, the first antigen-binding portion is a full-length antibody, the second antigen-binding portion and the third antigen-binding portion are both single-domain antibodies (VHH), the second antigen-binding portion replaces the Fab region on the side of the first antigen-binding portion, and the third antigen-binding portion is fused to the N-terminus of both heavy chains of the first antigen-binding portion, the first Fc region of the multispecific antigen-binding protein is knob-Fc, the second Fc region is hole-Fc.

In some embodiments, the first antigen-binding portion is a full-length antibody, the second antigen-binding portion and the third antigen-binding portion are both single-domain antibodies (VHH), the second antigen-binding portion replaces the Fab region on the side of the first antigen-binding portion, and the third antigen-binding portion is fused to the C-terminus of both heavy chains of the first antigen-binding portion, the first Fc region of the multispecific antigen-binding protein is knob-Fc, the second Fc region is hole-Fc.

In some embodiments, the first antigen-binding portion is a full-length antibody, the second antigen-binding portion is a scFv, the third antigen-binding portion is a single-domain antibody (VHH), the second antigen-binding portion replaces the Fab region on the side of the first antigen-binding portion, and the third antigen-binding portion is fused to the N-terminus of both heavy chains of the first antigen-binding portion, the first Fc region of the multispecific antigen-binding protein is knob-Fc, the second Fc region is hole-Fc.

In some embodiments, the first antigen-binding portion is a full-length antibody, the second antigen-binding portion is a scFv, the third antigen-binding portion is a single-domain antibody (VHH), the second antigen-binding portion replaces the Fab region on the side of the first antigen-binding portion, and the third antigen-binding portion is fused to the C-terminus of both heavy chains of the first antigen-binding portion, the first Fc region of the multispecific antigen-binding protein is knob-Fc, the second Fc region is hole-Fc.

In some embodiments, the first antigen-binding portion is a full-length antibody, the second antigen-binding portion is an Fv, the third antigen-binding portion is a single-domain antibody (VHH), the second antigen-binding portion replaces the Fv region of the first antigen-binding portion, and the third antigen-binding portion is fused to the N-terminus of both heavy chains of the first antigen-binding portion, the first Fc region of the multispecific antigen-binding protein is knob-Fc, the second Fc region is hole-Fc.

In some embodiments, the first antigen-binding portion is a full-length antibody, the second antigen-binding portion is an Fv, the third antigen-binding portion is a single-domain antibody (VHH), the second antigen-binding portion replaces the Fv region of the first antigen-binding portion, the VH and VL of the replacement portion are exchanged, and the third antigen-binding portion is fused to the N-terminus of both heavy chains of the first antigen-binding portion, the first Fc region of the multispecific antigen-binding protein is knob-Fc, the second Fc region is hole-Fc.

In some embodiments, the first antigen-binding portion is a full-length antibody, the second antigen-binding portion is an Fv, the third antigen-binding portion is a single-domain antibody (VHH), the second antigen-binding portion replaces the Fv region of the first antigen-binding portion, the CH1 and CL of the replacement portion are exchanged, and the third antigen-binding portion is fused to the N-terminus of both heavy chains of the first antigen-binding portion, the first Fc region of the multispecific antigen-binding protein is knob-Fc, the second Fc region is hole-Fc.

In some embodiments, the first antigen-binding portion is a full-length antibody, the second antigen-binding portion is an Fv, the third antigen-binding portion is a single-domain antibody (VHH), the second antigen-binding portion replaces the Fv region of the first antigen-binding portion, and the third antigen-binding portion is fused to the C-terminus of both heavy chains of the first antigen-binding portion, the first Fc region of the multispecific antigen-binding protein is knob-Fc, the second Fc region is hole-Fc.

In some embodiments, the first antigen-binding portion is a full-length antibody, the second antigen-binding portion is an Fv, the third antigen-binding portion is a single-domain antibody (VHH), the second antigen-binding portion replaces the Fv region of the first antigen-binding portion, the VH and VL of the replacement portion are exchanged, and the third antigen-binding portion is fused to the C-terminus of both heavy chains of the first antigen-binding portion, the first Fc region of the multispecific antigen-binding protein is knob-Fc, the second Fc region is hole-Fc.

In some embodiments, the first antigen-binding portion is a full-length antibody, the second antigen-binding portion is an Fv, the third antigen-binding portion is a single-domain antibody (VHH), the second antigen-binding portion replaces the Fv region of the first antigen-binding portion, the CH1 and CL of the replacement portion are exchanged, and the third antigen-binding portion is fused to the C-terminus of both heavy chains of the first antigen-binding portion, the first Fc region of the multispecific antigen-binding protein is knob-Fc, the second Fc region is hole-Fc.

In some embodiments, the first antigen-binding portion is a full-length antibody, the second antigen-binding portion and the third antigen-binding portion are both single-domain antibodies (VHH), the second antigen-binding portion is fused to the N-terminus of both heavy chains of the first antigen-binding portion, the third antigen-binding portion replaces the Fab region on the side of the first antigen-binding portion, and the first Fc region of the multispecific antigen-binding protein is knob-Fc, the second Fc region is hole-Fc.

In some embodiments, the first antigen-binding portion is a full-length antibody, the second antigen-binding portion and the third antigen-binding portion are both single-domain antibodies (VHH), the second antigen-binding portion is fused to the N-terminus of one of the heavy chains of the first antigen-binding portion, the third antigen-binding portion replaces the Fab region on the other side of the first antigen-binding portion, and the first Fc region of the multispecific antigen-binding protein is knob-Fc, the second Fc region is hole-Fc.

In some embodiments, the first antigen-binding portion is a full-length antibody, the second antigen-binding portion and the third antigen-binding portion are both single-domain antibodies (VHH), the second antigen-binding portion is fused to the N-terminus of one of the heavy chains of the first antigen-binding portion, the third antigen-binding portion replaces the Fab region on the same side of the first antigen-binding portion, and the first Fc region of the multispecific antigen-binding protein is knob-Fc, the second Fc region is hole-Fc.

In some embodiments, the first antigen-binding portion is a full-length antibody, the second antigen-binding portion and the third antigen-binding portion are both single-domain antibodies (VHH), the second antigen-binding portion is fused to the C-terminus of both heavy chains of the first antigen-binding portion, the third antigen-binding portion replaces the Fab region on the side of the first antigen-binding portion, and the first Fc region of the multispecific antigen-binding protein is knob-Fc, the second Fc region is hole-Fc.

In some embodiments, the first antigen-binding portion is a full-length antibody, the second antigen-binding portion and the third antigen-binding portion are both single-domain antibodies (VHH), the second antigen-binding portion is fused to the C-terminus of one of the heavy chains of the first antigen-binding portion, the third antigen-binding portion replaces the Fab region on the other side of the first antigen-binding portion, and the first Fc region of the multispecific antigen-binding protein is knob-Fc, the second Fc region is hole-Fc.

In some embodiments, the first antigen-binding portion is a full-length antibody, the second antigen-binding portion and the third antigen-binding portion are both single-domain antibodies (VHH), the second antigen-binding portion is fused to the C-terminus of one of the heavy chains of the first antigen-binding portion, the third antigen-binding portion replaces the Fab region on the same side of the first antigen-binding portion, and the first Fc region of the multispecific antigen-binding protein is knob-Fc, the second Fc region is hole-Fc.

In some embodiments, the first antigen-binding portion is a full-length antibody, the second antigen-binding portion is a single-domain antibody (VHH), the third antigen-binding portion is a scFv, the second antigen-binding portion is fused to the N-terminus of both heavy chains of the first antigen-binding portion, the third antigen-binding portion replaces the Fab region on the side of the first antigen-binding portion, and the first Fc region of the multispecific antigen-binding protein is knob-Fc, the second Fc region is hole-Fc.

In some embodiments, the first antigen-binding portion is a full-length antibody, the second antigen-binding portion is a single-domain antibody (VHH), the third antigen-binding portion is a scFv, the second antigen-binding portion is fused to the N-terminus of one of the heavy chains of the first antigen-binding portion, the third antigen-binding portion replaces the Fab region on the side of the first antigen-binding portion, and the first Fc region of the multispecific antigen-binding protein is knob-Fc, the second Fc region is hole-Fc.

In some embodiments, the first antigen-binding portion is a full-length antibody, the second antigen-binding portion is a single-domain antibody (VHH), the third antigen-binding portion is a scFv, the second antigen-binding portion is fused to the N-terminus of one of the heavy chains of the first antigen-binding portion, the third antigen-binding portion replaces the Fab region on the side where the first antigen-binding portion is fused to the second antigen-binding portion, and the first Fc region of the multispecific antigen-binding protein is knob-Fc, the second Fc region is hole-Fc.

In some embodiments, the first antigen-binding portion is a full-length antibody, the second antigen-binding portion is a single-domain antibody (VHH), the third antigen-binding portion is a scFv, the second antigen-binding portion is fused to the C-terminus of both heavy chains of the first antigen-binding portion, the third antigen-binding portion replaces the Fab region on the side of the first antigen-binding portion, and the first Fc region of the multispecific antigen-binding protein is knob-Fc, the second Fc region is hole-Fc.

In some embodiments, the first antigen-binding portion is a full-length antibody, the second antigen-binding portion is a single-domain antibody (VHH), the third antigen-binding portion is a scFv, the second antigen-binding portion is fused to the C-terminus of one of the heavy chains of the first antigen-binding portion, the third antigen-binding portion replaces the Fab region on the side of the first antigen-binding portion, and the first Fc region of the multispecific antigen-binding protein is knob-Fc, the second Fc region is hole-Fc.

In some embodiments, the first antigen-binding portion is a full-length antibody, the second antigen-binding portion is a single-domain antibody (VHH), the third antigen-binding portion is a scFv, the second antigen-binding portion is fused to the C-terminus of one of the heavy chains of the first antigen-binding portion, the third antigen-binding portion replaces the Fab region on the side where the first antigen-binding portion is fused to the second antigen-binding portion, and the first Fc region of the multispecific antigen-binding protein is knob-Fc, the second Fc region is hole-Fc.

In some embodiments, the first antigen-binding portion is a full-length antibody, the second antigen-binding portion is a single-domain antibody (VHH), the third antigen-binding portion is an Fv, the second antigen-binding portion is fused to the N-terminus of both heavy chains of the first antigen-binding portion, the third antigen-binding portion replaces the Fv region on the side of the first antigen-binding portion, and the first Fc region of the multispecific antigen-binding protein is knob-Fc, the second Fc region is hole-Fc.

In some embodiments, the first antigen-binding portion is a full-length antibody, the second antigen-binding portion is a single-domain antibody (VHH), the third antigen-binding portion is an Fv, the second antigen-binding portion is fused to the N-terminus of one of the heavy chains of the first antigen-binding portion, the third antigen-binding portion replaces the Fv region on the other side of the first antigen-binding portion, and the first Fc region of the multispecific antigen-binding protein is knob-Fc, the second Fc region is hole-Fc.

In some embodiments, the first antigen-binding portion is a full-length antibody, the second antigen-binding portion is a single-domain antibody (VHH), the third antigen-binding portion is an Fv, the second antigen-binding portion is fused to the N-terminus of one of the heavy chains of the first antigen-binding portion, the third antigen-binding portion replaces the Fv region on the same side of the first antigen-binding portion, and the first Fc region of the multispecific antigen-binding protein is knob-Fc, the second Fc region is hole-Fc.

In some embodiments, the first antigen-binding portion is a full-length antibody, the second antigen-binding portion is a single-domain antibody (VHH), the third antigen-binding portion is an Fv, the second antigen-binding portion is fused to the C-terminus of both heavy chains of the first antigen-binding portion, the third antigen-binding portion replaces the Fv region on the side of the first antigen-binding portion, and the first Fc region of the multispecific antigen-binding protein is knob-Fc, the second Fc region is hole-Fc.

In some embodiments, the first antigen-binding portion is a full-length antibody, the second antigen-binding portion is a single-domain antibody (VHH), the third antigen-binding portion is an Fv, the second antigen-binding portion is fused to the C-terminus of one of the heavy chains of the first antigen-binding portion, the third antigen-binding portion replaces the Fv region on the other side of the first antigen-binding portion, and the first Fc region of the multispecific antigen-binding protein is knob-Fc, the second Fc region is hole-Fc.

In some embodiments, the first antigen-binding portion is a full-length antibody, the second antigen-binding portion is a single-domain antibody (VHH), the third antigen-binding portion is an Fv, the second antigen-binding portion is fused to the C-terminus of one of the heavy chains of the first antigen-binding portion, the third antigen-binding portion replaces the Fv region on the same side of the first antigen-binding portion, and the first Fc region of the multispecific antigen-binding protein is knob-Fc, the second Fc region is hole-Fc.

In some embodiments, the second antigen-binding portion and the third antigen-binding portion are fused to the first antigen-binding portion by a linker.

In some embodiments, the linker is a peptide linker.

In some embodiments, the peptide linker is a GS linker or a mutant human IgG hinge.

In some embodiments, the GS linker is (G₄S)ₙ, (SG₄)ₙ or G₄(SG₄)ₙ linker.

In some embodiments, n is any natural number from 0-10.

In some embodiments, the peptide linker is (G₄S)ₙ.

In some embodiments, the tumor-associated antigen is selected from the group consisting of: GPC3, CD19, CD20 (MS4A1), CD22, CD24, CD30, CD33, CD38, CD40, CD123, CD133, CD138, CDK4, CEA, Claudin6, Claudin18.2, AFP, ALK, B7H3, BAGE protein, BCMA, BIRC5 (survivin), BIRC7, β-catenin, brc-ab1, BRCA1, BORIS, CA9, CA125, carbonic anhydrase IX, caspase-8, CALR, CCR5, CCR8, NA17, NKG2D, NY-BR1, NY-BR62, NY-BR85, NY-ESO1, OX40, p15, p53, PAP, PAX3, PAX5, PCTA-1, PLAC1, PRLR, PRAME, PSMA (FOLH1), RAGE proteins, cyclin-B1, CYP1B1, EGFR, EGFRVIII, ErbB2/Her2, ErbB3, ErbB4, ETV6-AML, EpCAM, EphA2, Fra-1, FOLR1, GAGE protein, GD2, GD3, GloboH, GM3, gp100, Her2, HLA/B-raf, HLA/k-ras, HLA/MAGE-A3, hTERT, IL13Rα2, LMP2, κ-Light, LeY, MAGE-1, MAGE-2, MAGE-3, MAGE-4, MAGE-6, MAGE-12, MART-1, mesothelin, ML-IAP, MOv-γ, Muc1, Muc2, Muc3, Muc4, Muc5, Muc16, MUM1, Ras, RGS5, Rho, ROR1, SART-1, SART-3, STEAP1, STEAP2, TAG-72, TGF-β, TNFR2, TMPRSS2, Thom-Knott's antigen, TRP-1, TRP-2, tyrosinase and urolytic protein-3, 5T4, hepatitis B surface antigen (HBSAG), tissue polypeptide antigen (TPA), carbohydrate antigen 153 (CA153), carbohydrate antigen 19-9 (CA19-9), carbohydrate antigen 724 (CA724), carbohydrate antigen 242 (CA242), carbohydrate antigen 50 (CA50), CYFRA21-1 (Cy211), neuron specific enolase (NSE), prostate-specific antigen (PSA), human chorionic gonadotropin (HCG), thyroglobulin (TG), ferritin (SF), β2-microglobulin (β2-MG), squamous cell antigen (SCC).

In some embodiments, the first antigen is CD24.

In some embodiments, the first antigen is Claudin18.2.

In some embodiments, the second antigen is selected from the group consisting of: NKP30, NKP46, CD16, NKP44, CD244, CD226, NKG2D, NKG2C, and KIR.

In some embodiments, the second antigen is NKP30.

In some embodiments, the second antigen is selected from the group consisting of: Vδ1T, Vδ2T, Vδ3T, γδTreg, γδT17, IFN-γ⁺γδT.

In some embodiments, the third antigen is selected from the group consisting of: EGF, FGF, FGF, FGF-α, FGF-β, VEGF, VEGFR, HDGF, HGF, HGFK1, NRP-1, ANG, Ang1, Ang2, PDGF, PDGFR, PIGF, TGF-α, TGF-β, TGF-β receptor, CD31, CD105, MCP-1, COX-2, AC133, Id2/Id3, IL-1α, IL-6, IL-1β, IL-8, CXCL5, MMP, and THBS, AREG, ET-1, AAMP, AGGF1, AMOT, ANGLPTL3, ANGPTL4, BTG1, NOS3, TNFSF12, VASH2, integrinαvβ3, α5β1, VE-cadherin, leptin, heparin, plasminogen activator, and plasminogen activator inhibitor-1.

In some embodiments, the third antigen is VEGF.

In some embodiments, the third antigen-binding portion is VEGFR.

In some embodiments, the first antigen is CD24, the second antigen is NKP30, the third antigen is VEGF.

In some embodiments, the first antigen is CD24, the second antigen is NKP30, the third antigen-binding portion is VEGFR.

In some embodiments, the first antigen is Claudin18.2, the second antigen is NKP30, the third antigen is VEGF.

In some embodiments, the first antigen is Claudin18.2, the second antigen is NKP30, the third antigen-binding portion is VEGFR.

In some embodiments, the Fab, scFab, F(ab′)2, Fv, dsFv, scFv, VH or VL structural domains of the first antigen-binding portion and/or the second antigen-binding portion and/or the third antigen-binding portion is a chimeric antibody, a fully human antibody or a humanized antibody.

In some embodiments, the single-domain antibody (VHH) of the first antigen-binding portion and/or the second antigen-binding portion and/or the third antigen-binding portion is a camel antibody, a shark antibody or a humanized single-domain antibody.

In some embodiments, the full-length antibody comprises an Fc fragment selected from the group consisting of: IgG, IgA, IgD, IgE, IgM and combinations thereof.

In some embodiments, the Fc fragment is selected from the group consisting of: IgG1, IgG2, IgG3, IgG4 and combinations thereof.

In some embodiments, the Fc fragment is a human Fc fragment.

In some embodiments, the full-length antibody has enhanced FcγR binding affinity compared with a corresponding antibody having a human IgG wild-type Fc fragment.

In some embodiments, the full-length antibody has reduced FcγR binding affinity compared with a corresponding antibody having a human IgG wild-type Fc fragment.

The present application also provides a pharmaceutical composition, wherein the pharmaceutical composition comprises a multispecific antigen-binding protein as described in any of the above embodiments and a pharmaceutically acceptable carrier.

The present application also provides use of the multispecific antigen-binding proteins described in any of the above embodiments or the pharmaceutical composition in the preparation of a drug for the treatment of cancer.

In some embodiments, the cancer is selected from the group consisting of: squamous cell carcinoma, myeloma, small cell lung cancer, non-small cell lung cancer (NSCLC), head and neck squamous cell carcinoma (HNSCC), chronic lymphocytic leukaemia (CLL), chronic myeloid leukaemia (CML), primary mediastinal large B-cell lymphoma, mantle cell lymphoma (MCL), small lymphocytic lymphoma (SLL), T-cell/histiocyte-rich large B-cell lymphoma, multiple myeloma, myeloid leukaemia-1 protein (Mcl-1), glioma, Hodgkin's lymphoma, non-Hodgkin's lymphoma, melanoma, glioblastoma, diffuse large B-cell lymphoma (DLBCL), follicular lymphoma, acute lymphoblastic leukemia (ALL), acute myeloid leukaemia (AML), myelodysplastic syndrome (MDS), gastric cancer, renal cancer, ovarian cancer, liver cancer, head and neck cancer, lymphoblastic leukaemia, lymphoblastic leukaemia, colorectal cancer, endometrial cancer, prostate cancer, central nervous system cancer, oesophageal cancer, malignant pleural mesothelioma, systemic light-chain amyloidosis, lymphoplasmacytic lymphoma, neuroendocrine neoplasms, merkel cell carcinoma, testicular cancer, skin cancer, thyroid cancer, melanoma, chondrosarcoma, neuroblastoma, pancreatic cancer, glioblastoma multiforme, gastric cancer, bone cancer, Ewing's sarcoma, cervical cancer, brain cancer, bladder cancer, hepatocellular tumor, breast cancer, colon cancer, hepatocellular carcinoma (HCC), clear cell renal cell carcinoma (RCC), head and neck cancer, throat cancer, and hepatobiliary cancer.

The present application also provides use of the multispecific antigen-binding protein described in any of the above embodiments and the pharmaceutical composition in the treatment of cancer.

In some embodiments, the cancer is selected from the group consisting of: squamous cell carcinoma, myeloma, small cell lung cancer, non-small cell lung cancer (NSCLC), head and neck squamous cell carcinoma (HNSCC), chronic lymphocytic leukaemia (CLL), chronic myeloid leukaemia (CML), primary mediastinal large B-cell lymphoma, mantle cell lymphoma (MCL), small lymphocytic lymphoma (SLL), T-cell/histiocyte-rich large B-cell lymphoma, multiple myeloma, myeloid leukaemia-1 protein (Mcl-1), glioma, Hodgkin's lymphoma, non-Hodgkin's lymphoma, melanoma, glioblastoma, diffuse large B-cell lymphoma (DLBCL), follicular lymphoma, acute lymphoblastic leukemia (ALL), acute myeloid leukaemia (AML), myelodysplastic syndrome (MDS), gastric cancer, renal cancer, ovarian cancer, liver cancer, head and neck cancer, lymphoblastic leukaemia, lymphoblastic leukaemia, colorectal cancer, endometrial cancer, prostate cancer, central nervous system cancer, oesophageal cancer, malignant pleural mesothelioma, systemic light-chain amyloidosis, lymphoplasmacytic lymphoma, neuroendocrine neoplasms, merkel cell carcinoma, testicular cancer, skin cancer, thyroid cancer, melanoma, chondrosarcoma, neuroblastoma, pancreatic cancer, glioblastoma multiforme, gastric cancer, bone cancer, Ewing's sarcoma, cervical cancer, brain cancer, bladder cancer, hepatocellular tumor, breast cancer, colon cancer, hepatocellular carcinoma (HCC), clear cell renal cell carcinoma (RCC), head and neck cancer, throat cancer, and hepatobiliary cancer.

Unless otherwise defined, all field terms, symbols and other scientific terms used herein are intended to have the meaning commonly understood by those skilled in the art to which the present invention belongs. In some instances, for the sake of clarity and/or for ease of reference, terms having commonly understood meanings are defined herein, and the inclusion of such definitions herein should not be construed to indicate a departure from what is commonly understood in the art.

The term “multispecific antigen-binding protein” refers to a protein molecule that specifically binds to two or more target antigens or target antigen epitopes. Protein molecules capable of binding specifically to two target antigens or target antigen epitopes are called bispecific antigen-binding proteins, and “bispecific binding protein” comprising an antibody or an antigen-binding fragment of an antibody (e.g., a single-chain antibody) is herein interchangeable with “bispecific antibody”.

The term “antigen-binding domain” refers to the portion of a multispecific protein molecule or an antibody molecule that has the ability to bind non-covalently, reversibly and specifically to an antigen. An antigen-binding domain may be a part of a ligand-binding domain that binds directly to an antigen or a domain comprising an antibody variable region that binds directly to an antigen. As used herein, the term “antigen-binding domain” encompasses an antibody fragment that retains the ability to bind antigen non-covalently, reversibly and specifically.

The term “antibody” encompasses an immunoglobulin molecule comprising four polypeptide chains, two heavy (H) chains and two light (L) chains, interconnected by disulphide bonds, as well as a multimer thereof (e.g. IgM). Each L chain is attached to the H chain by a covalent disulfide bond, while the two H chains are attached to each other by one or more disulfide bonds, depending on the H chain isoform. Each heavy chain has a variable region (abbreviated herein as VH) at the N-terminus, followed by a constant region. Each heavy chain contains a heavy chain variable region (abbreviated herein as HCVR or VH) and a heavy chain constant region. This heavy chain constant region contains three regions (structural domains), CH1, CH2 and CH3. Each light chain contains a light chain variable region (abbreviated in the text as LCVR or VL) and a light chain constant region. The light chain constant region contains one region (structural domain, CL1). The VH and VL regions can be further subdivided into highly variable regions, called complementary decision regions (CDR), with more conservative regions scattered in between, called framework region (FR, also known as backbone region, framing region). Each VH and VL is composed of three CDRs and four FRs, arranged from the amino-terminus to the carboxy-terminus in the following order: FR1, CDR1, FR2, CDR2, FR3, CDR3, FR4. Antibodies can be of different subclasses.

The term “antibody” includes, but is not limited to: monoclonal antibodies, fully human antibodies, humanized antibodies, camelid antibodies, chimeric antibodies, bispecific or multispecific antibodies, and anti-idiotype (anti-Id) antibodies (including, for example, anti-Id antibodies directed against the antibodies of this disclosure). These antibodies may belong to any isotype/type (e.g., IgG, IgE, IgM, IgD, IgA, and IgY) or subclass (e.g., IgG1, IgG2, IgG3, IgG4, IgA1, and IgA2).

The term “antigen-binding fragment” or “antigen-binding portion” refers to one or more portions of an antibody that retain the ability to bind the antigen bound by the antibody. Examples of “antigen-binding fragments” of an antibody include: (1) Fab fragments; (2) F(ab′)2 fragments; (3) Fd fragments, consisting of VH and CH1 structural domains; (4) Fv fragment; (5) dAb fragment, consisting of the VH structural domain; and (6) CDR, separated complementary determining regions.

In addition, although the two structural domains of the Fv fragment, VL and VH, are encoded by separate genes, recombinant methods can be used to link them by synthetic linkers, thereby enabling the production of a single protein chain for which the VL and VH regions are paired to form a monovalent molecule (referred to as single-chain Fv (scFv)). Such single-chain antibodies are also intended to be included in the term “antigen-binding fragment” of an antibody. Such antibody fragments are obtained using conventional techniques known to those skilled in the art, and the fragments are screened for utility in the same manner as for intact antibodies. The antigen-binding portion may be produced by recombinant DNA techniques or by enzymatic or chemical cleavage of the intact immunoglobulin. The antigen-binding fragment may also be incorporated into a single-chain molecule comprising a pair of tandem Fv fragments (VH—CH1-VH—CH1) which, together with a complementary light chain polypeptide form a pair of antigen-binding regions.

In some embodiments, the antigen-binding fragment of the antibody is in any configuration of variable and constant regions, where the variable and constant regions may be directly connected to each other or may be connected by complete or partial stranding or linker subregions. The stranded region may comprise at least 2 (e.g., 5, 10, 15, 20, 40, 60 or more) amino acids such that it creates flexible and semi-flexible linkages between adjacent variable and/or constant regions in a single polypeptide molecule. Further, the antigen-binding fragment of an antibody of the present invention may comprise homodimers or heterodimers (or other multimers) of any of the above listed variable and constant region configurations that are non-covalently linked to each other and/or linked (e.g., by a bisulfide bond) to one or more monomeric VH or VL regions.

The term “innate immune cells”, also known as innate immune cells, refers to cells that do not recognize pathogenic substances (eg, cancer cells, bacteria, viruses, and yeast) by expressing antibodies or TCRs on their cell surface, but instead express receptors (e.g., a receptor on its cell surface) or proteins that bind to the Fc region of an antibody that binds to a pathogen, and/or receptors that bind to a pattern-associated molecular pattern (PAMP) and/or a danger-associated molecular pattern (DAMP), where PAMP is associated with pathogens, DAMP is associated with damaged or transformed cells. Non-limiting examples of innate immune cells include mast cells, macrophages, neutrophils, dendritic cells, basophils, eosinophils, NK cells, and γδT cells. Other examples of innate immune cells are known in the art.

The term “murine antibody” refers to the fusion of B-cells from immunized mice with myeloma cells, followed by screening for murine hybrid fusion cells that can both proliferate indefinitely and secrete antibodies, and then perform screening, antibody preparation, and antibody purification.

The term “chimeric antibody” means an antibody molecule (or antigen-binding fragment thereof), wherein (1) the constant region, or portion thereof, is altered, replaced, or substituted so that the antigen-binding site (variable region) is connected to a constant region of a different or altered type, effector function, and/or species, or to a completely different molecules (e.g., enzymes, toxins, hormones, growth factors, drugs, etc.); or (2) the variable region or portion thereof is altered, displaced or replaced with a variable region having a different or altered antigenic specificity. For example, a mouse antibody can be modified by replacing its constant region with a constant region from a human immunoglobulin. As a result of being replaced by human constant regions, the chimeric antibody may retain its specificity for recognizing antigen while having reduced antigenicity in humans compared with the original mouse antibody.

The term “humanized antibody” means a chimeric antibody that contains amino acid residues derived from a human antibody sequence. The humanized antibody may contain some or all of the CDR or HVR from a non-human animal or synthetic antibody, while the framework and constant regions of the antibody contain amino acid residues derived from the human antibody sequence. Heterologous reactions induced by chimeric antibodies due to carrying a large number of heterologous protein components can be overcome. Such framing sequences can be obtained from public DNA databases comprising germline antibody gene sequences or from published references. In order to avoid the decrease in activity induced along with the decrease in immunogenicity, minimal reverse mutation or revertant mutation can be performed on the human antibody variable region frame sequences to maintain activity.

The term “fully human antibody” means an antibody having an amino acid sequence corresponding to an antibody produced by a human being or a human cell, or an amino acid sequence derived from a non-human source using a human antibody library or a human antibody coding sequence. If the antibody contains a constant region, the constant region is also derived from such human-like sequences, e.g. human germline sequences or mutated forms of human germline sequences, or antibodies containing a common framework sequence derived from human framework sequence analysis. Fully human antibodies explicitly exclude humanized antibodies.

The term “monoclonal antibody” refers to an antibody derived from a substantially homogeneous antibody population. Substantially homogeneous antibody populations contain antibodies that are substantially similar and bind the same epitopes, with the exception of variants that can typically occur during the production of monoclonal antibodies. Such variants are usually present only in small numbers. Monoclonal antibodies are highly specific for a single antigenic site. In contrast to polyclonal antibody preparations, which usually include different antibodies directed against different determinants (epitopes), each monoclonal antibody is directed against a single determinant on the antigen. In addition to their specificity, monoclonal antibodies have the advantage that they are synthesized by hybridoma culture and are not contaminated with other immunoglobulins. The modifier “monoclonal” denotes antibody characteristics such as antibodies obtained from a substantially homologous population of antibodies and should not be construed as requiring the generation of antibodies by any particular method. For example, monoclonal antibodies used in accordance with the present disclosure may be prepared by various techniques including, but not limited to, hybridoma methods, recombinant DNA methods, phage display methods, and methods using genetically engineered animals comprising all or part of the human immunoglobulin locus, as described herein, as well as other exemplary methods for the preparation of monoclonal antibodies.

The terms “full-length antibody”, “intact antibody” or “whole antibody” are used interchangeably to refer to an antibody that is substantially complete in its form as compared with an antibody fragment. In particular, full-length 4-chain antibodies include those having a heavy chain and a light chain including an Fc region. The constant domain may be a natural sequence constant domain or an amino acid sequence variant thereof. In some embodiments, the intact antibody may have one or more effector functions.

The terms “peptide” and “protein” are used interchangeably herein to refer to polymers of amino acid residues. The phrases also apply to amino acid polymers in which one or more amino acid residues are artificial chemical analogues of the corresponding naturally occurring amino acid, and to both naturally occurring and non-naturally occurring amino acid polymers. Unless otherwise indicated, a particular polypeptide sequence also implicitly encompasses conservatively modified variants thereof.

The term “amino acids” refers to twenty common naturally occurring amino acids. Naturally occurring amino acids include alanine (Ala; A), arginine (Arg; R), asparagine (Asn; N), aspartic acid (Asp; D), cysteine (Cys; C), glutamic acid (Glu; E), glutamine (Gln; Q), glycine (Gly; G), histidine (His; H), isoleucine (Ile; I), leucine (Leu; L), lysine (Lys; K), methionine (Met; M), phenylalanine (Phe; F), proline (Pro; P), serine (Ser; S), threonine (Thr; T), tryptophan (Trp; W), tyrosine (Tyr; Y) and valine (Val; V). In some embodiments, the term “amino acid” further includes a non-natural amino acid. Any suitable non-natural amino acid may be used. In some embodiments, the non-natural amino acid comprises a reactive portion for affixing the agent to the MIAC.

The term “Fc receptor” or “FcR” describes a receptor that binds the Fc region of an antibody. Preferred FcRs are natural sequence human FcRs. In addition, preferred FcRs are receptors that bind IgG antibodies (γ-receptors) and include receptors of the subclasses FcγRI, FcγRII, and FcγRIII, including allelic variants and alternatively spliced forms of these receptors, with the FcγRII receptor comprising FcγRIIA (“activating receptor”) and FcγRIIB (“inhibitory receptor”), which share the primary distinction of similar amino acid sequences in their cytoplasmic domains. The activating receptor FcγRIIA contains in its cytoplasmic domain an activation motif based on the immunoreceptor tyrosine (ITAM). The inhibitory receptor FcγRIIB contains an immunoreceptor tyrosine-based inhibitory motif (ITIM) in its cytoplasmic domain.

The term “Fc fragment” encompasses the carboxyl-terminal portion of the two H-chains held together by a disulphide bond. The effector function of the antibody is determined by the sequence of the Fc region, which is also recognized by the Fc receptor (FcR) present on some cell types.

The term “knob-Fc” refers to the replacement of an amino acid residue in the CH3 domain of the first subunit of the Fc structural domain with an amino acid residue having a larger side chain volume, thereby creating a bulge in the CH3 domain of the first subunit that can be localized in a depression in the CH3 domain of the second subunit. For example, by mutating serine T at position 366 of the CH3 of a heavy chain to tryptophan W, a protruding “knob”-like bulge is formed.

The term “hole-Fc” refers to the replacement of an amino acid residue in the CH3 domain of the second subunit of the Fc structural domain with an amino acid residue having a smaller side chain volume, thereby creating a depression in the CH3 domain of the second subunit within which a bulge in the CH3 domain of the first subunit can be positioned. For example, by mutating a serine T at position 366 to serine S, a leucine L at position 368 to alanine A, and an amino acid at position 407 from tyrosine Y to valine V or to alanine A in another heavy chain, a depressed “hole”-like depression is formed.

The term “Fab fragment” consists of the intact L-chain as well as the variable region structural domain (VH) of the H-chain and the first constant domain (CH1) of a heavy chain. Each Fab fragment is monovalent for antigen-binding, i.e., it has a single antigen-binding site. For example, Fab fragments can be produced recombinantly or by papain digestion of full-length antibodies.

The term “Fab′ fragment” differs from Fab fragment in that several additional residues are added to the carboxyl terminus of the CH1 domain, including one or more cysteines from the hinge region of the antibody. Fab′ can be produced by treating F(ab′)2, which specifically recognizes and binds to the antigen, with a reducing agent such as dithiothreitol.

The term “F(ab′)2 fragment” was originally created as a pair of Fab′ fragments with a hinge cysteine in between. The F(ab′)2 fragments can be produced recombinantly or by pepsin digestion of intact antibodies (which remove most of the Fc region while retaining portions of the intact hinge region). The F(ab′)2 fragment can be dissociated (into two Fab′ molecules) by treatment with a reducing agent such as β-mercaptoethanol.

The term “scFab” refers to a single-chain Fab fragment that is formed by introducing a peptide linker between the heavy chain variable domain (VH) and the light chain (CL) to form a single-chain Fab fragment (scFab).

The term “Fv” is the smallest antibody fragment that contains the complete antigen recognition and binding site. The fragment consists of a dimer formed by tight non-covalent binding of a heavy chain variable region domain to a light chain variable region domain. Folding of the two domains produces six highly variable loops (three loops from the H chain and three loops from the L chain) that contribute amino acid residues for antigen-binding and confer antigen-binding specificity to the antibody. However, even though the individual variable domains have the ability to recognize and bind antigen, their affinity is low compared with the full binding site.

The term “single chain Fv” or “sFv” or “scFv” fragment refers to an antibody fragment comprising the VH and VL structural domains of an antibody, wherein these structural domains are present in a single polypeptide chain. The Fv polypeptide may further comprise a polypeptide linker between the VH and VL structural domains, the polypeptide linker enabling scFv to form the structure desired for antigen-binding. The “scFv-Fc” fragment comprises scFv attached to the Fc structural domain. e.g., the Fc structural domain may be linked to the C-terminus of scFv. Depending on the orientation of the variable structural domain in the scFv (i.e., VH-VL or VL-VH), the Fc structural domain can be after VH or VL. The Fc structural domain can be any suitable Fc structural domain known in the art or described herein. In some embodiments, the Fc structural domain is an IgG1 Fc structural domain.

The term “dsFv” refers to disulfide bond stabilized Fv fragments. In dsFv, polypeptides in which one amino acid residue in each VH and VL is replaced by a cysteine residue are linked by a disulfide bond between the cysteine residues. To produce such molecules, one amino acid in the framework region of each of VH and VL is mutated to a cysteine, which in turn forms a stable interchain disulfide bond. Typically, the 44th position in VH and 100th position in VL mutated to cysteine. The term dsFv is described to cover both dsFv (molecules in which VH and VL are linked by an interchain disulfide bond rather than a linker peptide) or scdsFv (molecules in which VH and VL are linked by both a linker and an interchain disulfide bond), as known in the art.

The term “multispecific antibody” refers to an antibody that contains two or more antigen-binding domains capable of binding two or more different epitopes (e.g., two, three, four, or more different epitopes), which can be on the same or different antigens. Examples of multispecific antibodies include “bispecific antibodies” that bind two different epitopes, and “trispecific antibodies” that bind three different epitopes.

The term “fusion” refers to the joining of two amino acid sequences to form a new sequence, e.g., by means of a linker, to form a new synthetic protein or antibody.

The term “linker” or the use to connect the “L1” between two protein domains refers to a connective polypeptide sequence, which is used to connect protein structural domains with a certain degree of flexibility, and the use of the linker will not cause the original function of the protein structural domains to be lost.

The term “bis-antibody” refers to a small antibody fragment prepared by constructing a scFv fragment with a short linker (approximately 5-10 residues) between the VH and VL domains to enable inter-chain rather than intra-chain pairing of the V domains, resulting in a bivalent fragment, i.e., a fragment with two antigen-binding sites. Bispecific diabodies are heterodimers of two “crossed” scFv fragments in which the VH and VL domains of the two antibodies are present on different polypeptide chains.

The term “amino acid mutation” or “amino acid difference” means a mutation or alteration of an amino acid in a variant protein or polypeptide as compared with the original protein or polypeptide, including an insertion, deletion or substitution of one or more amino acids in the original protein or polypeptide.

The term “variable region” or “variable domain” of an antibody refers to the variable region of the light chain (VL) of an antibody or the variable region of the heavy chain (VH) of an antibody, individually or in combination. As known in the art, the variable regions of the heavy and light chains each comprise four framework regions (FRs) connected by three complementary decision regions (CDRs), also known as highly variable regions. The CDRs in each chain are tightly held together by the FRs and together with the CDRs from the other chain contribute to the formation of the antigen-binding site of the antibody. Heavy-chain-only antibodies from camelid species have a single heavy-chain variable region, which is referred to as “VHH”, and VHH is therefore a special type of VH.

The term “variable” refers to the fact that some segments of the variable domains vary widely in sequence from antibody to antibody. V structural domains mediate antigen-binding and define the specificity of a particular antibody for its particular antigen. However, variability is not uniformly distributed over the entire range of variable domains. Instead, it is concentrated in three segments called hypervariable regions (HVRs) within the light and heavy chain variable domains. The more highly conserved portion of the variable domain is called the framework region (FR). The variable domains of the natural heavy and light chains each contain four FR regions, most of which adopt a β-folded conformation and are connected by three HVRs, which form a ring connection and, in some cases, form part of the β-folded structure. The HVRs in each chain are held closely together by the FR regions and, together with the HVRs of the other chains, contribute to the formation of the antigen-binding site of the antibody. The constant domains are not directly implicated in antibody-antigen-binding, but exhibit various effector functions, such as involvement in antibody-dependent cytotoxicity of antibodies.

The term “complementary determining region” or “CDR” refers to one of the six highly variable regions within the variable structural domains of an antibody that primarily contribute to antigen-binding. One of the most commonly used definitions of the six CDRs is provided by Kabat E. A. et al, ((1991) Sequences of proteins of immunological interest. NIH Publication 91-3242). As used in some embodiments herein, CDRs can be defined in terms of Kabat's rule for CDR1, CDR2, and CDR3 of light chain variable structural domains (LCDR1, LCDR2, LCDR3), as well as CDR1, CDR2, and CDR3 of heavy chain variable structural domains (HCDR1, HCDR2, HCDR3).

The term “antigen-binding domain” refers to the portion of a molecule that has the ability to bind non-covalently, reversibly and specifically to an antigen. Exemplary antigen-binding domains include antigen-binding fragments and portions of immunoglobulin-based scaffolds and non-immunoglobulin-based scaffolds, wherein the scaffolds retain the ability to non-covalently, reversibly, and specifically bind to an antigen. As used herein, the term “antigen-binding domain” encompasses an antibody fragment that retains the ability to bind antigen non-covalently, reversibly and specifically.

The term “antibody constant region structural domains” refers to structural domains derived from the constant regions of the light and heavy chains of an antibody, including the CL and CH1, CH2, CH3 and CH4 structural domains derived from different classes of antibodies. The hinge region of an antibody that connects the CH1 and CH2 domains of the heavy chain does not fall within the scope of an “antibody constant region domain” as defined in the present application.

The term “tumor antigen” refers to substances, optionally proteins, produced by tumor cells, including “tumor-associated antigen” or “TAA” (which refers to proteins produced in tumor cells that are differentially expressed in the cancer as compared with the corresponding normal tissues), and “tumor-specific antigen” or “TSA” (which refers to tumor antigens that are produced in tumor cells and that are either specifically expressed or abnormally expressed in the cancer as compared with the corresponding normal tissues).

The term “tumor-associated antigen” or “TAA” refers to molecules (typically proteins, carbohydrates, lipids, or some combination thereof) that are expressed either exclusively or as fragments on the surface of cancerous cells, and that can be used to preferentially target pharmacological agents to cancerous cells. Non-limiting examples of “tumor-associated antigens” include, for example, CD19, CD20 (MS4A1), CD22, CD30, CD33, CD38, CD40, CD123, CD133, CD138, CDK4, CEA, Claudin 18.2, AFP, ALK, B7H3, BAGE protein, BCMA, BIRC5 (survivin), BIRC7, β-catenin, brc-ab1, BRCA1, BORIS, CA9, CA125, carbonic anhydrase IX, caspase-8, CALR, CCR5, NA17, NKG2D, NY-BR1, NY-BR62, NY-BR85, NY-ESO1, OX40, p15, p53, PAP, PAX3, PAX5, PCTA-1, PLAC1, PRLR, PRAME, PSMA (FOLH1), RAGE proteins, cyclin-B1, CYP1B1, EGFR, EGFRVIII, ErbB2/Her2, ErbB3, ErbB4, ETV6-AML, EpCAM, EphA2, Fra-1, FOLR1, GAGE proteins (e.g. GAGE-1, GAGE-2), GD2, GD3, GloboH, glypican-3, GM3, gp100, Her2, HLA/B-raf, HLA/k-ras, HLA/MAGE-A3, hTERT, IL13Rα2, LMP2, κ-Light, LeY, MAGE proteins (e.g. MAGE-1, MAGE-2, MAGE-3, MAGE-4, MAGE-6 and MAGE-12) MART-1, mesothelin, ML-IAP, MOv-γ, Muc1, Muc2, Muc3, Muc4, Muc5, Muc16 (CA-125), MUM1, Ras, RGS5, Rho, ROR1, SART-1, SART-3, STEAP1, STEAP2, TAG-72, TGF-β, TMPRSS2, Thompson-nouvelle antigen (Tn), TRP-1, TRP-2, tyrosinase and urolytic protein-3, 5T4 (Trophoblast glycoprotein).

Non limiting examples of “tumor specific antigen” or “TSA” include, for example, hepatitis B surface antigen (HBSAG), tissue polypeptide antigen (TPA), carbohydrate antigen 153 (CA153), carbohydrate antigen 19-9 (CA19-9), carbohydrate antigen 724 (CA724), carbohydrate antigen 242 (CA242), carbohydrate antigen 50 (CA50), CYFRA21-1 (Cy211), neuron specific enolase (NSE), prostate-specific antigen (PSA), human chorionic gonadotropin (HCG), thyroglobulin (TG), ferritin (SF), β2-microglobulin (β2-MG), squamous cell antigen (SCC).

The term “epitope” or “antigenic determinant cluster” refers to the portion of an antigen that is bound by an antibody (or its antigen-binding fragment). Epitopes typically consist of surface-accessible amino acid residues and/or sugar side chains, and can have specific three-dimensional structural features as well as specific charge features. The difference between conformational and non-conformational epitopes is that binding to the former, but not the latter, is lost in the presence of denaturing solvents. Epitopes may include amino acid residues that are directly involved in binding and other amino acid residues that are not directly involved in binding.

The terms “specifically binds”, “selectively binds” and “specifically binds” refer to a measurable and reproducible interaction, such as binding, between a target and an antibody, including here biological molecules in the presence of a heterogeneous population of, the presence of the target is determined. For example, an antibody that binds or specifically binds a target (which may be an epitope) is an antibody that binds this target with greater affinity, more readily and/or for a longer duration than it binds other targets. Typically, the antibody binds with an affinity (KD) of about less than 10-8 M, such as about less than 10-9 M, 10-10 M, 10-11 M or less.

The term “affinity” refers to the strength of the sum of non-covalent interactions between a single binding site of a molecule (e.g., the antigen-binding module of MIAC) and its binding partner (e.g., the antigen). Within each antigenic site, the variable region of the antibody “arm” interacts with the antigen at multiple amino acid sites through weak non-covalent forces; the greater the interaction, the stronger the affinity. Unless otherwise indicated, “binding affinity” as used herein refers to the intrinsic binding affinity that reflects a 1:1 interaction between members of a binding pair (e.g., antibody and antigen). The affinity of a molecule X for its partner Y can generally be expressed as a dissociation constant (Kd). The affinity can be measured by commonly used methods known in the art, such as by using surface plasmon resonance (SPR) techniques (e.g., instrumentation) or biolayer interferometry (e.g., instrumentation).

The term “high affinity” generally refers to an antibody or antigen-binding fragment having a KD of 1E-9M or less (e.g., 1E-10M or less, 1E-11M or less, 1E-12M or less, 1E-13M or less, 1E-14M or less, etc.).

The term “KD” refers to the dissociation equilibrium constant for a particular antibody-antigen interaction. Typically, the antibody binds the antigen at a dissociation equilibrium constant (KD) of less than about 1E-8M, such as less than about 1E-9M, 1E-10M or 1E-11M or less, for example, in a BIACORE instrument using surface plasmon resonance (SPR) technology. The smaller the KD value, the greater the affinity.

The term “antibody effector function” refers to those biological activities that are attributable to the Fc region of an antibody (either the natural sequence Fc region or the amino acid sequence variant Fc region) and that vary with antibody isotype. Examples of antibody effector functions include C1q binding and complement-dependent cytotoxicity; Fc receptor binding; antibody-dependent cell-mediated cytotoxicity (ADCC); phagocytosis; down-regulation of cell-surface receptors (e.g., B cell receptors); and B cell activation. “Reduced or minimized” antibody effector function means that the antibody effector function is reduced by at least 50% (or 60%, 65%, 70%, 75%, 80%, 85%, 90%, 95%, 96%, 97%, 98%, 99%) as compared with wild type or unmodified antibodies. Assays of antibody effector function can be readily determined and measured by one of ordinary skill in the art.

The term “effector cell” is a leukocyte that expresses one or more FcRs and performs an effector function. In one aspect, the effector cell expresses at least FcγRIII and performs an ADCC effector function. Examples of human leukocytes mediating ADCC include peripheral blood mononuclear cells (PBMC), natural killer (NK) cells, monocytes, cytotoxic T cells, and neutrophilic leukocytes. Effector cells can be isolated from natural sources (e.g., blood). Effector cells are generally lymphocytes associated with the effector phase and are used to produce cytokines (helper T-cells), kill cells infected with pathogens (cytotoxic T-cells) or secrete antibodies (differentiated B-cells).

The term “antibody-dependent cell-mediated cytotoxicity” or “ADCC” refers to a form of cytotoxicity in which binding to secreted Ig on Fc receptors (FcRs) present on certain cytotoxic cells (e.g., natural killer (NK) cells, neutrophilic leukocytes, and macrophages) allows these cytotoxic effector cells to specifically bind to antigen-bearing target cells and subsequently kill the target cells with cytotoxins. The antibodies “arm” cytotoxic cells are required to kill target cells by this mechanism. The major cells that mediate ADCC (NK cells) express only FcγRIII, whereas monocytes express FcγRI, FcγRII, and FcγRIII. In order to evaluate the ADCC activity of a molecule of interest, an in vitro ADCC assay can be performed. Effector cells useful for such assays include peripheral blood mononuclear cells (PBMC) and natural killer (NK) cells.

The term “complement dependent cytotoxicity” or “CDC” refers to the lysis of target cells in the presence of complement. Activation of the classical complement pathway is triggered by the binding of the first component of the complement system (C1q) to an antibody (of the appropriate subclass) bound to its cognate antigen. To evaluate complement activation, CDC assays can be performed, e.g., as described in Gazzano-Santoro et al, J. Immunol. Methods 202: 163 (1996). Antibody variants having altered amino acid sequences in the Fc region and increased or decreased C1q binding capacity are described in U.S. Pat. No. 6,194,551B1 and WO99/51642. The contents of those patent publications are expressly incorporated herein by reference.

The term “single-domain antibody” or “VHH” refers to a single antigen-binding peptide that contains only one variable heavy chain region (VHH).

The term “nucleic acid molecule” refers to DNA and RNA molecules. Nucleic acid molecules may be single-stranded or double-stranded, but preferably double-stranded DNA, and nucleic acids are “efficiently linked” when placed in a functional relationship with another nucleic acid sequence.

The term “vector” refers to a construct capable of delivering one or more target genes or sequences and preferably expressing them in a host cell. The vector may be a plasmid, phage, transposon, cosmid, chromosome, virus or virion. One type of vector can be integrated into the genome of the host cell upon introduction into the host cell and thereby replicate with the host genome (e.g., non-episomal mammalian vectors). Another type of vector is capable of replicating autonomously in the host cell into which it is introduced (e.g., bacterial vectors with bacterial replication start points and episomal mammalian vectors). Another specific type of vector capable of directing the expression of the expressible foreign nucleic acid to which they are operatively linked is often referred to as an “expression vector”. Expression vectors typically have a control sequence that drives the expression of the expressible foreign nucleic acid. Simpler vectors known as “transcription vectors” are only capable of being transcribed rather than translated: they are replicated rather than expressed in target cells. The term “vector” covers all types of vectors, regardless of their function. Vectors capable of directing the expression of the expressible nucleic acids to which they are operatively linked are commonly referred to as “expression vectors”. In this specification, “plasmid” and “vector” are used interchangeably, as plasmids are the most commonly used form of vector.

The term “host cell” refers to a cell system that can be engineered to produce a target protein, protein fragment or peptide. Host cells include, but are not limited to cultured cells, such as mammalian cultured cells derived from rodent (rat, mouse, guinea pig or hamster) such as CHO, BHK, NSO, SP2/0, YB2/0; Human cells, such as HEK293F cells, HEK293T cells; Or human tissue or hybridoma cells, yeast cells, insect cells (e.g., S2 cells), bacterial cells (e.g., Escherichia coli (E. coli) cells), and cells contained within transgenic animals or cultured tissues. The term covers not only a particular subject cell, but also the progeny of such a cell. Because certain modifications can occur in the progeny due to mutations or environmental influences, the progeny may not be identical to the parental cells, but are still included within the term “host cell”.

The terms “administering” and “treating”, when applied to an animal, human, experimental subject, cell, tissue, organ or biological fluid, refer to the contact of an exogenous drug, therapeutic, diagnostic or composition with an animal, human, subject, cell, tissue, organ or biological fluid. “Administering” and “treating” may refer to, for example, therapeutic, pharmacokinetic, diagnostic, research and experimental methods. Treatment of cells includes contact of a reagent with a cell, and contact of a reagent with a fluid, wherein the fluid is in contact with a cell. “Administering” and “treating” also mean in vitro and ex vivo treatment of, for example, a cell by a reagent, diagnostic, binding composition or by another cell. “Treating” when applied to human, veterinary medicine or research subjects means therapeutic treatment, prophylactic or preventive measures, research and diagnostic applications.

The term “treatment” means causing a desired or beneficial effect in the mammal with the disease condition. A desirable or beneficial effect may include a reduction in the frequency or severity of one or more symptoms of the disease (i.e. tumor growth and/or metastasis, or other effects mediated by the number and/or activity of immune cells, etc.), or a cessation or inhibition of further progression of the disease, affliction or condition. In the context of treating cancer in a mammal, desirable or beneficial effects may include inhibiting further growth or spread of cancer cells, causing death of the cancer cells, inhibiting recurrence of the cancer, alleviating pain associated with the cancer, or improving survival of the mammal. The effect may be subjective or objective.

The term “effective amount” refers to the amount of a drug, compound or pharmaceutical composition necessary to achieve any one or more beneficial or desired therapeutic outcomes. For prophylactic use, beneficial or desired outcomes include the elimination or reduction of risk, the reduction of severity or the delay of the onset of the condition, including the disease, its complications, its complications and the biochemical, histological and/or behavioural symptoms of intermediate pathological phenotypes presented during the progression of the disease. For therapeutic applications, beneficial or desired results include clinical outcomes such as reducing the incidence of various target antigen-related conditions of the present disclosure or ameliorating one or more symptoms of the conditions, reducing the dosage of other agents required to treat the condition, enhancing the efficacy of another agent, and/or delaying the progression of a disease associated with a target antigen of the present disclosure in a patient.

The term “exogenous” refers to substances produced outside organisms, cells, or the human body according to the situation.

The term “endogenous” refers to substances produced in cells, organisms or the human body according to the situation.

The terms “homology” or “percentage of amino acid sequence identity (%)” are used interchangeably herein to refer to sequence similarity between two polynucleotide sequences or between two polypeptides. When positions in two comparative sequences are both occupied by the same base or amino acid monomer subunit, e.g., if every position in two DNA molecules is occupied by an adenine, then the molecules are homologous at that position. The percentage of homology between two sequences is a function of the number of matching or homologous positions shared by the two sequences divided by the number of positions compared×100. For example, when sequences are best compared, if there are 6 matches or homologues at 10 positions in the two sequences, then the two sequences are 60% homologous; if there are 95 matches or homologues at 100 positions in the two sequences, then the two sequences are 95% homologous. Generally, comparisons are made when comparing two sequences to give the maximum percentage homology. The comparison performed to determine the percentage amino acid sequence identity may be achieved by various methods within the skill of the art, for example, using publicly available computer software, such as BLAST, BLAST-2, ALIGN, or MEGALIGN™ (DNASTAR) software. A person skilled in the art may determine the appropriate parameters for measuring the comparison, including any algorithms required to achieve maximum comparison over the full length of the sequences being compared.

The term “monovalent” refers to an antigen-binding molecule with a single antigen-binding structural domain.

The term “bivalent” refers to an antigen-binding molecule with two antigen-binding structural domains. The structural domains may be identical or different. Thus, a bivalent antigen-binding molecule may be monospecific or bispecific.

The term “trivalent” refers to an antigen-binding molecule with three antigen-binding domains.

The term “tetravalent” refers to an antigen-binding molecule with four antigen-binding domains.

The term “pentavalent” refers to an antigen-binding molecule with five antigen-binding domains.

The term “hexavalent” refers to an antigen-binding molecule with six antigen-binding domains.

The term “isolated” antibody is an antibody that has been identified, isolated and/or recovered from components of the environment in which it was produced. Preferably, the isolated polypeptide does not bind to all other components from its environment of production. Contaminating components of its generating environment are materials that would normally interfere with the research, diagnostic or therapeutic use of the antibody, and may include enzymes, hormones and other proteinaceous or nonproteinaceous solutes. In preferred embodiments, the polypeptide will be purified (1) to greater than 95% by weight of antibody, as determined by, for example, the lowry method, and in some embodiments, to greater than 99% by weight; (2) to an extent sufficient to obtain at least 15 residues of the N-terminus or internal amino acid sequence, by using a rotary-cup sequencer; or (3) to homogeneity, using coomassie blue or preferably a silver stain by SDS-PAGE under non-reducing or reducing conditions. The isolated antibody comprises an antibody in situ within the recombinant cell, since at least one component of the antibody's natural environment will not be present. Ordinarily, however, the isolated polypeptide or antibody will be prepared by at least one purification step.

The terms “optional” or “optionally” imply that the event or circumstance subsequently described may but need not occur, and the description includes occasions when the event or circumstance does or does not occur. For example, “optionally comprising 1-3 antibody heavy chain variable regions” means that an antibody heavy chain variable region of a particular sequence may, but need not, be present.

The term “pharmaceutical preparation” refers to a formulation that is in a dosage form that permits effective exertion of the biological activity of the active ingredient and that contains no additional components that are unacceptably toxic to the subject to whom the formulation is administered. Such preparations are sterile. “Sterile” preparations are sterile or free of all viable microorganisms and their spores.

The term “pharmaceutically acceptable carrier” refers to any inactive substance suitable for use in formulations for the delivery of binding molecules. The carrier may be an anti-adhesive, adhesive, coating, disintegrant, filler or diluent, preservative (such as an antioxidant agent, antibacterial agent or antifungal agent), sweetener, a delayed absorption agent, wetting agent, emulsifier, buffers, etc. Examples of suitable pharmaceutically acceptable carriers include water, ethanol, polyols (such as glycerol, propylene glycol, polyethylene glycol, etc.), dextrose, vegetable oils (such as olive oil), saline, buffers, buffered saline, and isotonic agents such as sugars, polyols, sorbitol, and sodium chloride.

The term “immune checkpoint molecule” means a molecule of the immune system that up-regulates or down-regulates signals. A “stimulatory immune checkpoint molecule” or “co-stimulatory molecule” is an immune checkpoint molecule that up-regulates signals in the immune system. An “inhibitory immune checkpoint molecule” is an immune checkpoint molecule that down-regulate signals in the immune system.

The term “cancer” refers to a disease characterized by the uncontrolled (and often rapid) growth of abnormal cells. Cancer cells can spread to other parts of the body locally or through the bloodstream and lymphatic system. Examples of cancers include, but are not limited to, carcinoma, lymphoma, blastoma, sarcoma, and leukaemia or lymphoid malignancy. More specific examples of such cancers include squamous cell carcinoma, myeloma, small cell lung cancer, non-small cell lung cancer (NSCLC), head and neck squamous cell carcinoma (HNSCC), chronic lymphocytic leukaemia (CLL), chronic myeloid leukaemia (CML), primary mediastinal large B-cell lymphoma, mantle cell lymphoma (MCL), small lymphocytic lymphoma (SLL), T-cell/histiocyte-rich large B-cell lymphoma, multiple myeloma, myeloid leukaemia-1 protein (Mcl-1), glioma, Hodgkin's lymphoma, non-Hodgkin's lymphoma, diffuse large B-cell lymphoma (DLBCL), follicular lymphoma, acute lymphoblastic leukemia (ALL), acute myeloid leukaemia (AML), myelodysplastic syndrome (MDS), Gastric cancer, renal cancer, ovarian cancer, liver cancer, lymphoblastic leukaemia, lymphoblastic leukaemia, colorectal cancer, endometrial cancer, prostate cancer, central nervous system cancer, oesophageal cancer, malignant pleural mesothelioma, systemic light-chain amyloidosis, lymphoplasmacytic lymphoma, myelodysplastic syndrome, myeloproliferative neoplasms, neuroendocrine neoplasms, merkel cell carcinoma, testicular cancer, skin cancer, thyroid cancer, melanoma, chondrosarcoma, neuroblastoma, pancreatic cancer, glioblastoma multiforme, gastric cancer, bone cancer, Ewing's sarcoma, cervical cancer, brain cancer, bladder cancer, hepatocellular tumor, breast cancer, colon cancer, hepatocellular carcinoma (HCC), clear cell renal cell carcinoma (RCC), head and neck cancer, throat cancer, hepatobiliary cancer.

The multi-specific antigen-binding protein of the present invention generates synergistic anti-tumor effect through multi-target combinations. On the one hand, the multispecific antigen-binding protein targets tumor antigens; on the other hand, NK cells can be specifically activated by the multispecific antigen-binding protein in the tumor microenvironment; at the same time, the immunosuppression of the tumor microenvironment can be relieved.

The multi-specific antigen-binding protein of the present invention can increase tumor microenvironment effector cells and relieve immunosuppression in the tumor microenvironment while exerting tumor targeting effect.

### BRIEF DESCRIPTION OF THE DRAWINGS

FIG. 1 depicts an exemplary multispecific antigen-binding protein, a full-length antibody that can specifically recognize the first antigen is fused with the second antigen-binding portion and the third antigen-binding portion, the second antigen-binding portion is a single-domain antibody (VHH), the third antigen-binding portion is a single-domain antibody (VHH) or scFv or an antigen receptor, as an example in the figure, the third antigen-binding portion is a single-domain antibody (VHH), the second antigen-binding portion is fused to the N-terminus of both light chains of the full-length antibody, and the third antigen-binding portion is fused to the C-terminus of both heavy chains of the full-length antibody.

FIG. 2 depicts an exemplary multispecific antigen-binding protein, a full-length antibody that can specifically recognize the first antigen is fused with the second antigen-binding portion and the third antigen-binding portion, the second antigen-binding portion is a single-domain antibody (VHH), the third antigen-binding portion is a single-domain antibody (VHH) or scFv or an antigen receptor, as an example in the figure, the third antigen-binding portion is a single-domain antibody (VHH), the second antigen-binding portion is fused to the N-terminus of both heavy chains of the full-length antibody, and the third antigen-binding portion is fused to the C-terminus of both heavy chains of the full-length antibody.

FIG. 3 depicts an exemplary multispecific antigen-binding protein, a full-length antibody that can specifically recognize the first antigen is fused with the second antigen-binding portion and the third antigen-binding portion, the second antigen-binding portion is a single-domain antibody (VHH), the third antigen-binding portion is a single-domain antibody (VHH) or scFv or an antigen receptor, as an example in the figure, the third antigen-binding portion is a single-domain antibody (VHH), the second antigen-binding portion is fused to the C-terminus of both light chains of the full-length antibody, and the third antigen-binding portion is fused to the C-terminus of both heavy chains of the full-length antibody.

FIG. 4 depicts an exemplary multispecific antigen-binding protein, a full-length antibody that can specifically recognize the first antigen is fused with the second antigen-binding portion and the third antigen-binding portion, the second antigen-binding portion is a single-domain antibody (VHH), the third antigen-binding portion is a single-domain antibody (VHH) or scFv or an antigen receptor, as an example in the figure, the third antigen-binding portion is a single-domain antibody (VHH), the second antigen-binding portion is fused to the N-terminus of one of the light chains of the full-length antibody, the VH and VL of the Fab region of the full-length antibody fused to the second antigen-binding portion are exchanged, the third antigen-binding portion is fused to the C-terminus of both heavy chains of the full-length antibody, and the first Fc region of the multispecific antigen-binding protein is knob-Fc, the second Fc region is hole-Fc.

FIG. 5 depicts an exemplary multispecific antigen-binding protein, a full-length antibody that can specifically recognize the first antigen is fused with the second antigen-binding portion and the third antigen-binding portion, the second antigen-binding portion is a single-domain antibody (VHH), the third antigen-binding portion is a single-domain antibody (VHH) or scFv or an antigen receptor, as an example in the figure, the third antigen-binding portion is a single-domain antibody (VHH), the second antigen-binding portion is fused to the N-terminus of one of the heavy chains of the full-length antibody, the third antigen-binding portion is fused to the C-terminus of both heavy chains of the full-length antibody, and the first Fc region of the multispecific antigen-binding protein is knob-Fc, the second Fc region is hole-Fc.

FIG. 6 depicts an exemplary multispecific antigen-binding protein, a full-length antibody that can specifically recognize the first antigen is fused with the second antigen-binding portion and the third antigen-binding portion, the second antigen-binding portion is a single-domain antibody (VHH), the third antigen-binding portion is a single-domain antibody (VHH) or scFv or an antigen receptor, as an example in the figure, the third antigen-binding portion is a single-domain antibody (VHH), the second antigen-binding portion is fused to the C-terminus of one of the light chains of the full-length antibody, the VH and VL of the Fab region of the full-length antibody fused to the second antigen-binding portion are exchanged, the third antigen-binding portion is fused to the C-terminus of both heavy chains of the full-length antibody, and the first Fc region of the multispecific antigen-binding protein is knob-Fc, the second Fc region is hole-Fc.

FIG. 7 depicts an exemplary multispecific antigen-binding protein, a full-length antibody that can specifically recognize the first antigen is fused with the second antigen-binding portion and the third antigen-binding portion, the second antigen-binding portion is a single-domain antibody (VHH), the third antigen-binding portion is a single-domain antibody (VHH) or scFv or an antigen receptor, as an example in the figure, the third antigen-binding portion is a single-domain antibody (VHH), the second antigen-binding portion is fused to the C-terminus of both heavy chains of the full-length antibody, and the third antigen-binding portion is fused to the N-terminus of both heavy chains of the full-length antibody.

FIG. 8 depicts an exemplary multispecific antigen-binding protein, a full-length antibody that can specifically recognize the first antigen is fused with the second antigen-binding portion and the third antigen-binding portion, the second antigen-binding portion is a single-domain antibody (VHH), the third antigen-binding portion is a single-domain antibody (VHH) or scFv or an antigen receptor, as an example in the figure, the third antigen-binding portion is a single-domain antibody (VHH), the second antigen-binding portion is fused to the C-terminus of one of the heavy chains of the full-length antibody, the third antigen-binding portion is fused to the N-terminus of both heavy chains of the full-length antibody, and the first Fc region of the multispecific antigen-binding protein is knob-Fc, the second Fc region is hole-Fc.

FIG. 9 depicts an exemplary multispecific antigen-binding protein, a full-length antibody that can specifically recognize the first antigen is fused with the second antigen-binding portion and the third antigen-binding portion, the second antigen-binding portion is a single-domain antibody (VHH), the third antigen-binding portion is a single-domain antibody (VHH) or scFv or an antigen receptor, as an example in the figure, the third antigen-binding portion is a single-domain antibody (VHH), the second antigen-binding portion replaces the Fab region on the side of the full-length antibody, the third antigen-binding portion is fused to the N-terminus of both heavy chains of the full-length antibody, and the first Fc region of the multispecific antigen-binding protein is knob-Fc, the second Fc region is hole-Fc.

FIG. 10 depicts an exemplary multispecific antigen-binding protein, a full-length antibody that can specifically recognize the first antigen is fused with the second antigen-binding portion and the third antigen-binding portion, the second antigen-binding portion is a single-domain antibody (VHH), the third antigen-binding portion is a single-domain antibody (VHH) or scFv or an antigen receptor, as an example in the figure, the third antigen-binding portion is a single-domain antibody (VHH), the second antigen-binding portion replaces the Fab region on the side of the full-length antibody, the third antigen-binding portion is fused to the C-terminus of both heavy chains of the full-length antibody, and the first Fc region of the multispecific antigen-binding protein is knob-Fc, the second Fc region is hole-Fc.

FIG. 11 depicts an exemplary multispecific antigen-binding protein, a full-length antibody that can specifically recognize the first antigen is fused with the second antigen-binding portion and the third antigen-binding portion, the second antigen-binding portion is a scFv, the third antigen-binding portion is a single-domain antibody (VHH) or scFv or an antigen receptor, as an example in the figure, the third antigen-binding portion is a single-domain antibody (VHH), the second antigen-binding portion replaces the Fab region on the side of the full-length antibody, the third antigen-binding portion is fused to the N-terminus of both heavy chains of the full-length antibody, and the first Fc region of the multispecific antigen-binding protein is knob-Fc, the second Fc region is hole-Fc.

FIG. 12 depicts an exemplary multispecific antigen-binding protein, a full-length antibody that can specifically recognize the first antigen is fused with the second antigen-binding portion and the third antigen-binding portion, the second antigen-binding portion is a scFv, the third antigen-binding portion is a single-domain antibody (VHH) or scFv or an antigen receptor, as an example in the figure, the third antigen-binding portion is a single-domain antibody (VHH), the second antigen-binding portion replaces the Fab region on the side of the full-length antibody, the third antigen-binding portion is fused to the C-terminus of both heavy chains of the full-length antibody, and the first Fc region of the multispecific antigen-binding protein is knob-Fc, the second Fc region is hole-Fc.

FIG. 13 depicts an exemplary multispecific antigen-binding protein, a full-length antibody that can specifically recognize the first antigen is fused with the second antigen-binding portion and the third antigen-binding portion, the second antigen-binding portion is an Fv, the third antigen-binding portion is a single-domain antibody (VHH) or scFv or an antigen receptor, as an example in the figure, the third antigen-binding portion is a single-domain antibody (VHH), the second antigen-binding portion replaces the Fv region of the full-length antibody, the third antigen-binding portion is fused to the N-terminus of both heavy chains of the full-length antibody, and the first Fc region of the multispecific antigen-binding protein is knob-Fc, the second Fc region is hole-Fc.

FIG. 14 depicts an exemplary multispecific antigen-binding protein, a full-length antibody that can specifically recognize the first antigen is fused with the second antigen-binding portion and the third antigen-binding portion, the second antigen-binding portion is an Fv, the third antigen-binding portion is a single-domain antibody (VHH) or scFv or an antigen receptor, as an example in the figure, the third antigen-binding portion is a single-domain antibody (VHH), the second antigen-binding portion replaces the Fv region of the full-length antibody, the VH and VL of the replacement portion are exchanged, the third antigen-binding portion is fused to the N-terminus of both heavy chains of the full-length antibody, and the first Fc region of the multispecific antigen-binding protein is knob-Fc, the second Fc region is hole-Fc.

FIG. 15 depicts an exemplary multispecific antigen-binding protein, a full-length antibody that can specifically recognize the first antigen is fused with the second antigen-binding portion and the third antigen-binding portion, the second antigen-binding portion is an Fv, the third antigen-binding portion is a single-domain antibody (VHH) or scFv or an antigen receptor, as an example in the figure, the third antigen-binding portion is a single-domain antibody (VHH), the second antigen-binding portion replaces the Fv region of the full-length antibody, the CH1 and CL of the replacement portion are exchanged, the third antigen-binding portion is fused to the N-terminus of both heavy chains of the full-length antibody, and the first Fc region of the multispecific antigen-binding protein is knob-Fc, the second Fc region is hole-Fc.

FIG. 16 depicts an exemplary multispecific antigen-binding protein, a full-length antibody that can specifically recognize the first antigen is fused with the second antigen-binding portion and the third antigen-binding portion, the second antigen-binding portion is an Fv, the third antigen-binding portion is a single-domain antibody (VHH) or scFv or an antigen receptor, as an example in the figure, the third antigen-binding portion is a single-domain antibody (VHH), the second antigen-binding portion replaces the Fv region of the full-length antibody, the third antigen-binding portion is fused to the C-terminus of both heavy chains of the full-length antibody, and the first Fc region of the multispecific antigen-binding protein is knob-Fc, the second Fc region is hole-Fc.

FIG. 17 depicts an exemplary multispecific antigen-binding protein, a full-length antibody that can specifically recognize the first antigen is fused with the second antigen-binding portion and the third antigen-binding portion, the second antigen-binding portion is an Fv, the third antigen-binding portion is a single-domain antibody (VHH) or scFv or an antigen receptor, as an example in the figure, the third antigen-binding portion is a single-domain antibody (VHH), the second antigen-binding portion replaces the Fv region of the full-length antibody, the VH and VL of the replacement portion are exchanged, the third antigen-binding portion is fused to the C-terminus of both heavy chains of the full-length antibody, and the first Fc region of the multispecific antigen-binding protein is knob-Fc, the second Fc region is hole-Fc.

FIG. 18 depicts an exemplary multispecific antigen-binding protein, a full-length antibody that can specifically recognize the first antigen is fused with the second antigen-binding portion and the third antigen-binding portion, the second antigen-binding portion is an Fv, the third antigen-binding portion is a single-domain antibody (VHH) or scFv or an antigen receptor, as an example in the figure, the third antigen-binding portion is a single-domain antibody (VHH), the second antigen-binding portion replaces the Fv region of the full-length antibody, the CH1 and CL of the replacement portion are exchanged, the third antigen-binding portion is fused to the C-terminus of both heavy chains of the full-length antibody, and the first Fc region of the multispecific antigen-binding protein is knob-Fc, the second Fc region is hole-Fc.

FIG. 19 depicts an exemplary multispecific antigen-binding protein, a full-length antibody that can specifically recognize the first antigen is fused with the second antigen-binding portion and the third antigen-binding portion, the second antigen-binding portion is a single-domain antibody (VHH), the third antigen-binding portion is a single-domain antibody (VHH) or scFv or an antigen receptor, as an example in the figure, the third antigen-binding portion is a single-domain antibody (VHH), the second antigen-binding portion is fused to the N-terminus of both heavy chains of the full-length antibody, the third antigen-binding portion replaces the Fab region on the side of the full-length antibody, and the first Fc region of the multispecific antigen-binding protein is knob-Fc, the second Fc region is hole-Fc.

FIG. 20 depicts an exemplary multispecific antigen-binding protein, a full-length antibody that can specifically recognize the first antigen is fused with the second antigen-binding portion and the third antigen-binding portion, the second antigen-binding portion is a single-domain antibody (VHH), the third antigen-binding portion is a single-domain antibody (VHH) or scFv or an antigen receptor, as an example in the figure, the third antigen-binding portion is a single-domain antibody (VHH), the second antigen-binding portion is fused to the N-terminus of one of the heavy chains of the full-length antibody, the third antigen-binding portion replaces the Fab region on the other side of the full-length antibody, and the first Fc region of the multispecific antigen-binding protein is knob-Fc, the second Fc region is hole-Fc.

FIG. 21 depicts an exemplary multispecific antigen-binding protein, a full-length antibody that can specifically recognize the first antigen is fused with the second antigen-binding portion and the third antigen-binding portion, the second antigen-binding portion is a single-domain antibody (VHH), the third antigen-binding portion is a single-domain antibody (VHH) or scFv or an antigen receptor, as an example in the figure, the third antigen-binding portion is a single-domain antibody (VHH), the second antigen-binding portion is fused to the N-terminus of one of the heavy chains of the full-length antibody, the third antigen-binding portion replaces the Fab region on the same side of the full-length antibody, and the first Fc region of the multispecific antigen-binding protein is knob-Fc, the second Fc region is hole-Fc.

FIG. 22 depicts an exemplary multispecific antigen-binding protein, a full-length antibody that can specifically recognize the first antigen is fused with the second antigen-binding portion and the third antigen-binding portion, the second antigen-binding portion is a single-domain antibody (VHH), the third antigen-binding portion is a single-domain antibody (VHH) or scFv or an antigen receptor, as an example in the figure, the third antigen-binding portion is a single-domain antibody (VHH), the second antigen-binding portion is fused to the C-terminus of both heavy chains of the full-length antibody, the third antigen-binding portion replaces the Fab region on the side of the full-length antibody, and the first Fc region of the multispecific antigen-binding protein is knob-Fc, the second Fc region is hole-Fc.

FIG. 23 depicts an exemplary multispecific antigen-binding protein, a full-length antibody that can specifically recognize the first antigen is fused with the second antigen-binding portion and the third antigen-binding portion, the second antigen-binding portion is a single-domain antibody (VHH), the third antigen-binding portion is a single-domain antibody (VHH) or scFv or an antigen receptor, as an example in the figure, the third antigen-binding portion is a single-domain antibody (VHH), the second antigen-binding portion is fused to the C-terminus of one of the heavy chains of the full-length antibody, the third antigen-binding portion replaces the Fab region on the other side of the full-length antibody, and the first Fc region of the multispecific antigen-binding protein is knob-Fc, the second Fc region is hole-Fc.

FIG. 24 depicts an exemplary multispecific antigen-binding protein, a full-length antibody that can specifically recognize the first antigen is fused with the second antigen-binding portion and the third antigen-binding portion, the second antigen-binding portion is a single-domain antibody (VHH), the third antigen-binding portion is a single-domain antibody (VHH) or scFv or an antigen receptor, as an example in the figure, the third antigen-binding portion is a single-domain antibody (VHH), the second antigen-binding portion is fused to the C-terminus of one of the heavy chains of the full-length antibody, the third antigen-binding portion replaces the Fab region on the same side of the full-length antibody, and the first Fc region of the multispecific antigen-binding protein is knob-Fc, the second Fc region is hole-Fc.

FIG. 25 depicts an exemplary multispecific antigen-binding protein, a full-length antibody that can specifically recognize the first antigen is fused with the second antigen-binding portion and the third antigen-binding portion, the second antigen-binding portion is a single-domain antibody (VHH), the third antigen-binding portion is a scFv, the second antigen-binding portion is fused to the N-terminus of both heavy chains of the full-length antibody, the third antigen-binding portion replaces the Fab region on the side of the full-length antibody, and the first Fc region of the multispecific antigen-binding protein is knob-Fc, the second Fc region is hole-Fc.

FIG. 26 depicts an exemplary multispecific antigen-binding protein, a full-length antibody that can specifically recognize the first antigen is fused with the second antigen-binding portion and the third antigen-binding portion, the second antigen-binding portion is a single-domain antibody (VHH), the third antigen-binding portion is a scFv, the second antigen-binding portion is fused to the N-terminus of one of the heavy chains of the full-length antibody, the third antigen-binding portion replaces the Fab region on the other side of the full-length antibody, and the first Fc region of the multispecific antigen-binding protein is knob-Fc, the second Fc region is hole-Fc.

FIG. 27 depicts an exemplary multispecific antigen-binding protein, a full-length antibody that can specifically recognize the first antigen is fused with the second antigen-binding portion and the third antigen-binding portion, the second antigen-binding portion is a single-domain antibody (VHH), the third antigen-binding portion is a scFv, the second antigen-binding portion is fused to the N-terminus of one of the heavy chains of the full-length antibody, the third antigen-binding portion replaces the Fab region on the side where the full-length antibody is fused to the second antigen-binding portion, and the first Fc region of the multispecific antigen-binding protein is knob-Fc, the second Fc region is hole-Fc.

FIG. 28 depicts an exemplary multispecific antigen-binding protein, a full-length antibody that can specifically recognize the first antigen is fused with the second antigen-binding portion and the third antigen-binding portion, the second antigen-binding portion is a single-domain antibody (VHH), the third antigen-binding portion is a scFv, the second antigen-binding portion is fused to the C-terminus of both heavy chains of the full-length antibody, the third antigen-binding portion replaces the Fab region on the side of the full-length antibody, and the first Fc region of the multispecific antigen-binding protein is knob-Fc, the second Fc region is hole-Fc.

FIG. 29 depicts an exemplary multispecific antigen-binding protein, a full-length antibody that can specifically recognize the first antigen is fused with the second antigen-binding portion and the third antigen-binding portion, the second antigen-binding portion is a single-domain antibody (VHH), the third antigen-binding portion is a scFv, the second antigen-binding portion is fused to the C-terminus of one of the heavy chains of the full-length antibody, the third antigen-binding portion replaces the Fab region on the other side of the full-length antibody, and the first Fc region of the multispecific antigen-binding protein is knob-Fc, the second Fc region is hole-Fc.

FIG. 30 depicts an exemplary multispecific antigen-binding protein, a full-length antibody that can specifically recognize the first antigen is fused with the second antigen-binding portion and the third antigen-binding portion, the second antigen-binding portion is a single-domain antibody (VHH), the third antigen-binding portion is a scFv, the second antigen-binding portion is fused to the C-terminus of one of the heavy chains of the full-length antibody, the third antigen-binding portion replaces the Fab region on the side where the full-length antibody is fused to the second antigen-binding portion, and the first Fc region of the multispecific antigen-binding protein is knob-Fc, the second Fc region is hole-Fc.

FIG. 31 depicts an exemplary multispecific antigen-binding protein, a full-length antibody that can specifically recognize the first antigen is fused with the second antigen-binding portion and the third antigen-binding portion, the second antigen-binding portion is a single-domain antibody (VHH), the third antigen-binding portion is an Fv, the second antigen-binding portion is fused to the N-terminus of both heavy chains of the full-length antibody, the third antigen-binding portion replaces the Fv region on the side of the full-length antibody, and the first Fc region of the multispecific antigen-binding protein is knob-Fc, the second Fc region is hole-Fc.

FIG. 32 depicts an exemplary multispecific antigen-binding protein, a full-length antibody that can specifically recognize the first antigen is fused with the second antigen-binding portion and the third antigen-binding portion, the second antigen-binding portion is a single-domain antibody (VHH), the third antigen-binding portion is an Fv, the second antigen-binding portion is fused to the N-terminus of one of the heavy chains of the full-length antibody, the third antigen-binding portion replaces the Fv region on the other side of the full-length antibody, and the first Fc region of the multispecific antigen-binding protein is knob-Fc, the second Fc region is hole-Fc.

FIG. 33 depicts an exemplary multispecific antigen-binding protein, a full-length antibody that can specifically recognize the first antigen is fused with the second antigen-binding portion and the third antigen-binding portion, the second antigen-binding portion is a single-domain antibody (VHH), the third antigen-binding portion is an Fv, the second antigen-binding portion is fused to the N-terminus of one of the heavy chains of the full-length antibody, the third antigen-binding portion replaces the Fv region on the same side of the full-length antibody, and the first Fc region of the multispecific antigen-binding protein is knob-Fc, the second Fc region is hole-Fc.

FIG. 34 depicts an exemplary multispecific antigen-binding protein, a full-length antibody that can specifically recognize the first antigen is fused with the second antigen-binding portion and the third antigen-binding portion, the second antigen-binding portion is a single-domain antibody (VHH), the third antigen-binding portion is an Fv, the second antigen-binding portion is fused to the C-terminus of both heavy chains of the full-length antibody, the third antigen-binding portion replaces the Fv region on the side of the full-length antibody, and the first Fc region of the multispecific antigen-binding protein is knob-Fc, the second Fc region is hole-Fc.

FIG. 35 depicts an exemplary multispecific antigen-binding protein, a full-length antibody that can specifically recognize the first antigen is fused with the second antigen-binding portion and the third antigen-binding portion, the second antigen-binding portion is a single-domain antibody (VHH), the third antigen-binding portion is an Fv, the second antigen-binding portion is fused to the C-terminus of one of the heavy chains of the full-length antibody, the third antigen-binding portion replaces the Fv region on the other side of the full-length antibody, and the first Fc region of the multispecific antigen-binding protein is knob-Fc, the second Fc region is hole-Fc.

FIG. 36 depicts an exemplary multispecific antigen-binding protein, a full-length antibody that can specifically recognize the first antigen is fused with the second antigen-binding portion and the third antigen-binding portion, the second antigen-binding portion is a single-domain antibody (VHH), the third antigen-binding portion is an Fv, the second antigen-binding portion is fused to the C-terminus of one of the heavy chains of the full-length antibody, the third antigen-binding portion replaces the Fv region on the same side of the full-length antibody, and the first Fc region of the multispecific antigen-binding protein is knob-Fc, the second Fc region is hole-Fc.

FIG. 37 shows the binding activity of the constructed antibodies CNVR-A and CNVR-B to NKP30 protein.

FIG. 38 shows the binding activity of the constructed antibody CNVR-C to NKP30 protein.

FIG. 39 shows the binding activity of the constructed antibodies CNVR-A, CNVR-B and CNVR-C to VEGFA protein.

FIG. 40 shows the binding of the Claudin18.2 antibody to PANC-1-Claudin18.2 cells detected by flow cytometry.

FIG. 41 shows the binding of the NC group to PANC-1-Claudin18.2 cells detected by flow cytometry.

FIG. 42 shows the binding of the NKP30 monoclonal antibody to PANC-1-Claudin18.2 cells detected by flow cytometry.

FIG. 43 shows the binding of the IgG1 isotype control to PANC-1-Claudin18.2 cells detected by flow cytometry.

FIG. 44 shows the binding of the constructed antibody CNV-C1 to PANC-1-Claudin18.2 cells detected by flow cytometry.

FIG. 45 shows the binding of the constructed antibody CNVR-A to PANC-1-Claudin18.2 cells detected by flow cytometry.

FIG. 46 shows the binding of the constructed antibody CNVR-B to PANC-1-Claudin18.2 cells detected by flow cytometry.

FIG. 47 shows the binding of the constructed antibody CNVR-C to PANC-1-Claudin18.2 cells detected by flow cytometry.

FIG. 48 shows the specific killing of PANC-1-Claudin18.2 cells by NK cells mediated by the constructed antibodies.

### DETAILED DESCRIPTION

The technical embodiments of the present invention are described in detail below in conjunction with the accompanying drawings. It should be understood that the specific embodiments described here are only used to explain the present invention, and are not intended to limit the present invention. The scope of the present application is not limited by these embodiments, but the scope of the patent application shall prevail.

### EXAMPLE 1 ACQUISITION AND OPTIMIZATION OF NUCLEOTIDE SEQUENCES

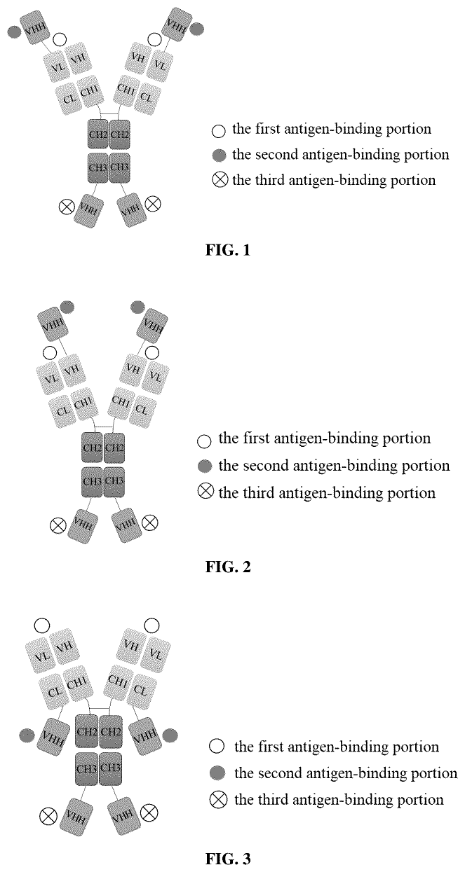

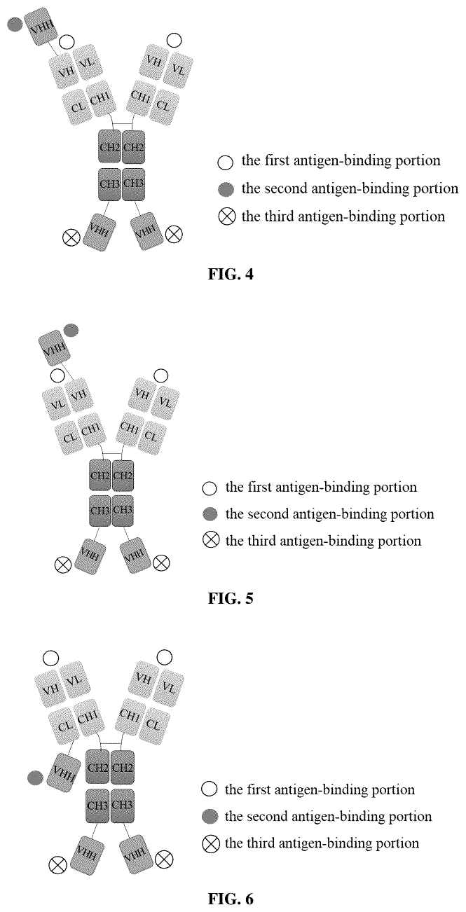

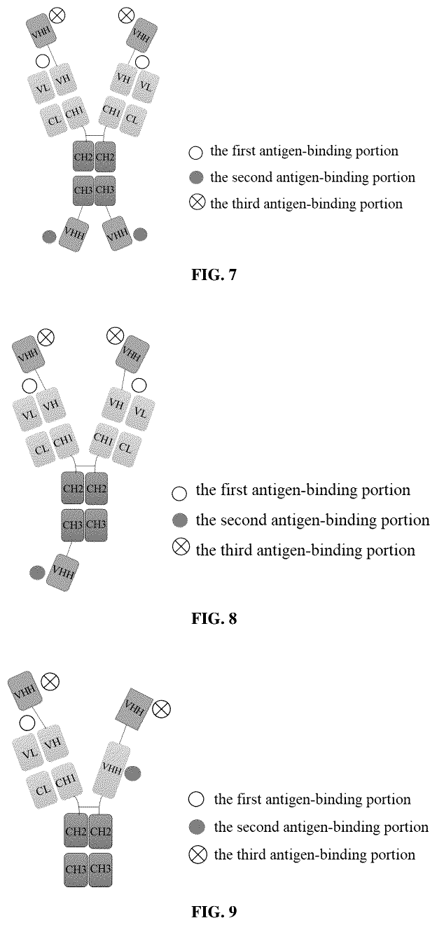

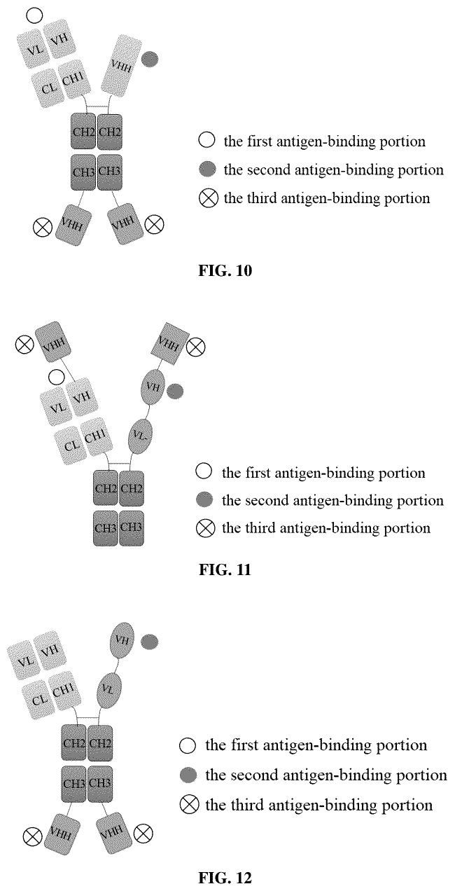

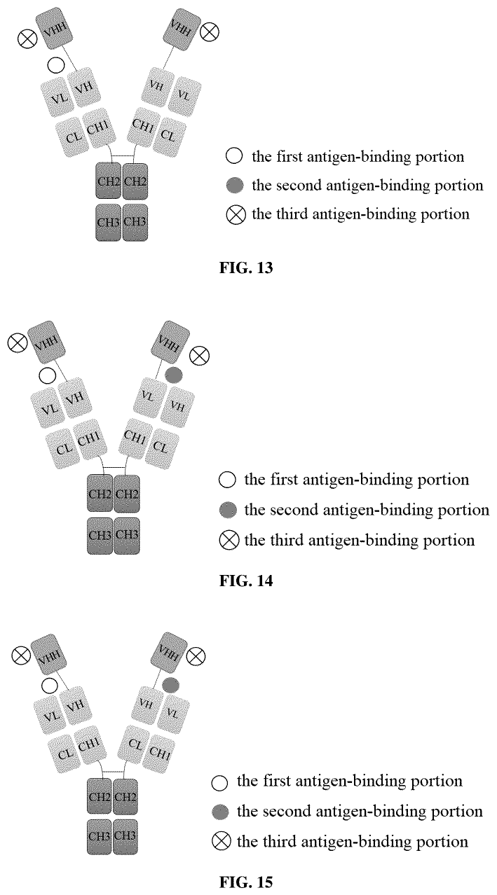

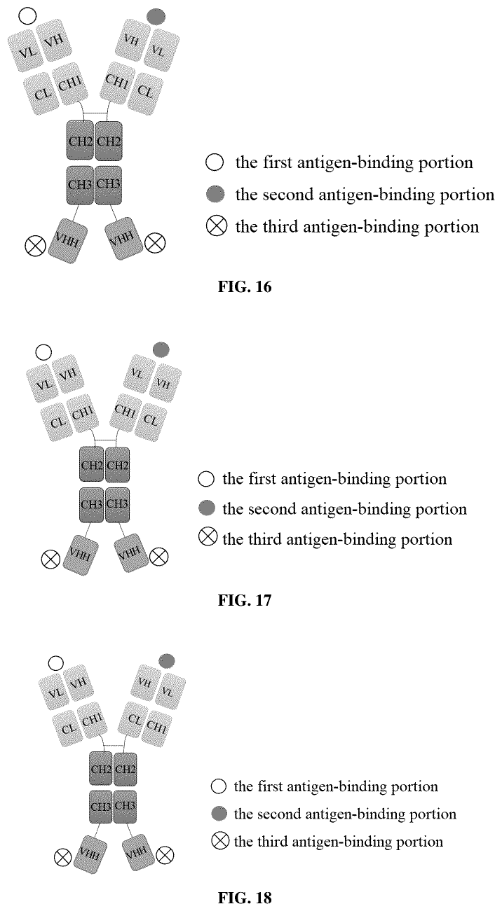

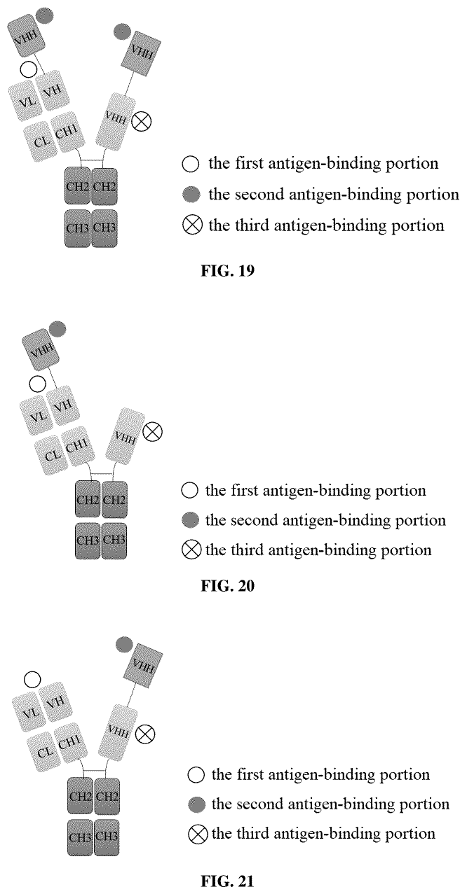

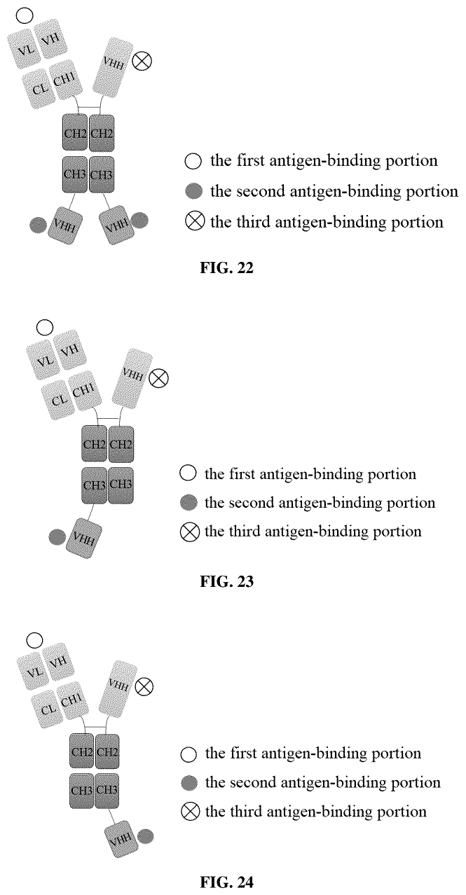

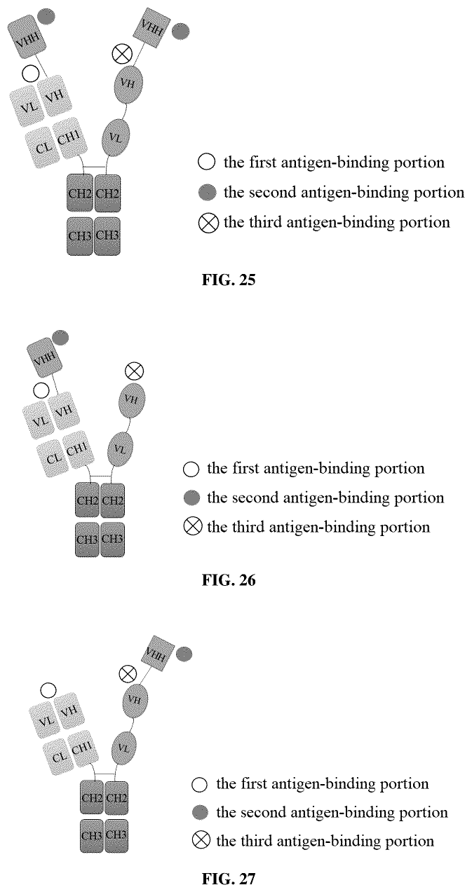

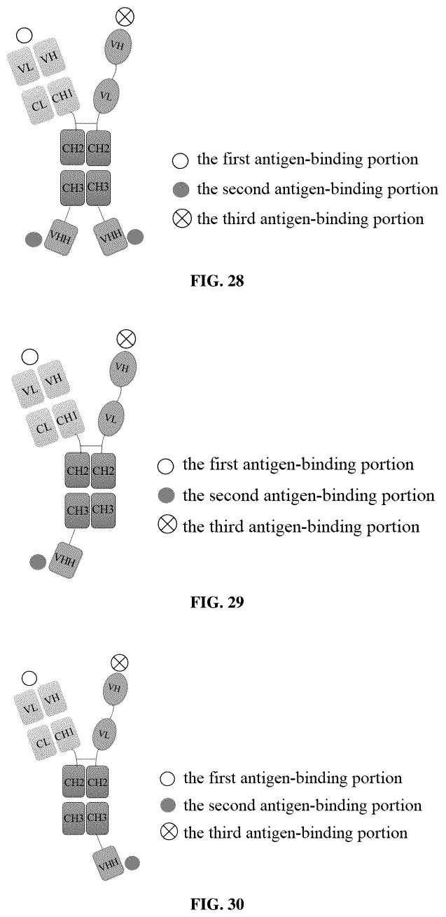

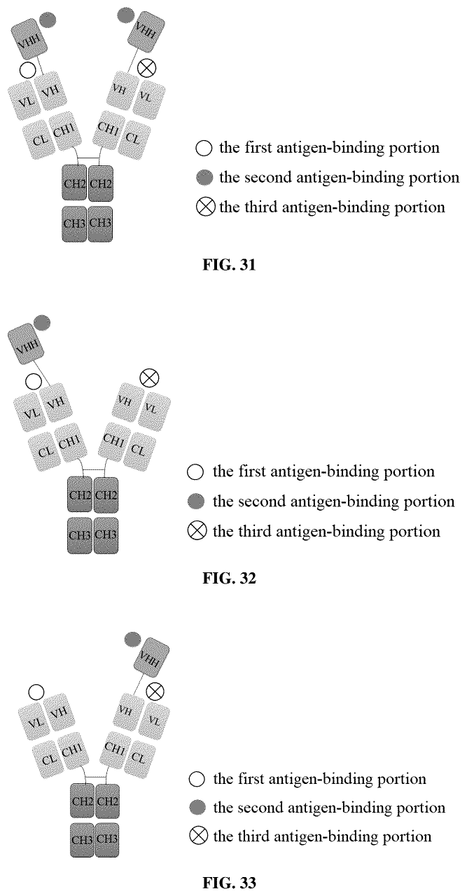

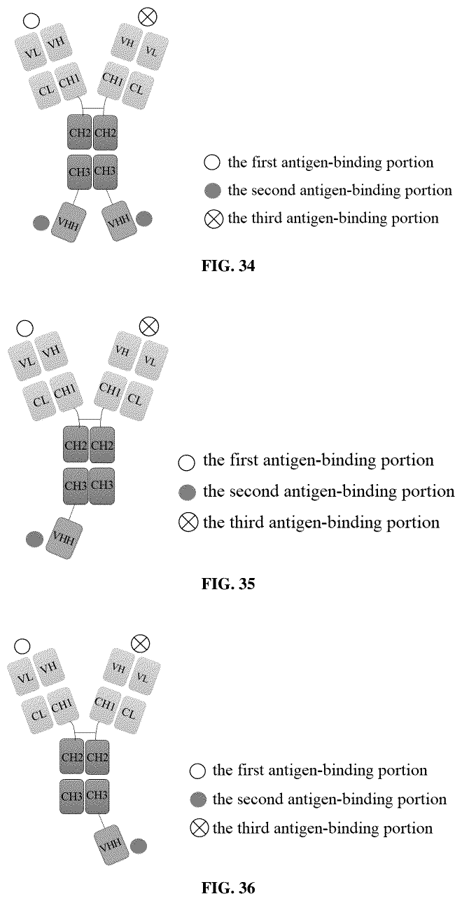

Against Claudin18.2, NKP30 targets and VEGF, trifunctional antibodies were constructed according to the six structures of FIGS. 1-6. In the trifunctional antibodies constructed in this embodiment, the first antigen-binding portion targets Claudin18.2; the second antigen-binding portion targets NKP30 in the form of VHH; and the third antigen-binding portion targets VEGF in the form of VEGF receptor or scFv. If the antigen-binding fragment targeting VEGF is VEGF receptor, it is VEGFR1, which is named as CNVR-A to CNVR-F in sequence. If the form of targeting VEGF is scFv, it is named as CNV-A to CNV-F in sequence, the sequence of VH and VL in scFv from the N-terminus to the C-terminus of antibody is VH-VL, and it is named as CNV-A1 to CNV-F1 in sequence, if it is VL-VH sequence, it is named as CNV-A2 to CNV-F2 in sequence.

The amino acid sequence information of the light chain and heavy chain of antibodies is shown in Table 1. VEGFR1 or VEGF scFv was fused to the C-terminus of the two heavy chains (i.e., after CH3) through a front linker, and NKP30 was a nano-humanized antibody fused to the corresponding position followed by linker fusion. According to needs, the Fc of the antibody amino acid sequence was adjusted to other IgG types, such as IgG1, etc., and further amino acid mutations of the desired form were designed in each heavy chain, thus obtaining the amino acid sequences of the target antibodies, and the sequences used and the combinations of the amino acid sequences of the constructed antibodies are shown in Tables 1 and 2.

TABLE 1
Sequences

| | Designation | | | | | |
|---|---|---|---|---|---|---|
| | | | | Claudin18.2 | Claudin18.2 | |
| | NKP30 VHH | VEGF VH | VEGF VL | VH | VL | VEGFR1 |
| SEQ ID No. | 1 | 2 | 3 | 4 | 5 | 6 |
| | Designation | | | | | |
| | IgG1 CH1-HINGE- | IgG1 T366W | IgG1 Y407A | | | |
| | CH2-CH3 | knob | hole | IgG1 CL | Linker | / |
| SEQ ID No. | 7 | 8 | 9 | 10 | 11 | / |

TABLE 2
Sequence combinations of constructed antibodies

| | Light | Heavy | Heavy | Light |
|---|---|---|---|---|
| Structure | chain 1 | chain 1 | chain 2 | Chain 2 |
| code | SEQ ID No. | SEQ ID No. | SEQ ID No. | SEQ ID No. |
| CNVR-A | 13 | 17 | 17 | 13 |
| CNVR-B | 12 | 18 | 18 | 12 |
| CNVR-C | 14 | 17 | 17 | 14 |
| CNVR-D | 15 | 19 | 20 | 12 |
| CNVR-E | 12 | 21 | 20 | 12 |
| CNVR-F | 16 | 19 | 20 | 12 |
| CNV-A1 | 13 | 22 | 22 | 13 |
| CNV-B1 | 12 | 23 | 23 | 12 |
| CNV-C1 | 14 | 22 | 22 | 14 |
| CNV-D1 | 15 | 24 | 25 | 12 |
| CNV-E1 | 12 | 26 | 25 | 12 |
| CNV-F1 | 16 | 24 | 25 | 12 |
| CNV-A2 | 13 | 27 | 27 | 13 |
| CNV-B2 | 12 | 28 | 28 | 12 |
| CNV-C2 | 14 | 27 | 27 | 14 |
| CNV-D2 | 15 | 29 | 30 | 12 |
| CNV-E2 | 12 | 31 | 30 | 12 |
| CNV-F2 | 16 | 29 | 30 | 12 |

Each of the above-mentioned target amino acid sequences was converted into nucleotide sequences, and a series of parameters that may affect antibody expression in mammalian cells were optimized, such as codon preference, GC content (that is, the ratio of guanine G and cytosine C in the 4 bases of DNA), CpG islands (that is, regions with a higher density of CpG dinucleotides in the genome), mRNA secondary structure, splicing sites, premature PolyA sites, internal Chi sites (a short piece of DNA in the genome, the probability of homologous recombination near the site increased), ribosome binding sites, RNA unstable sequences, inverted repeats and restriction enzyme sites that may interfere with cloning, etc. At the same time, related sequences that may improve translation efficiency were added, such as Kozak sequence and SD sequence. The heavy chain genes and the light chain genes encoding the above-mentioned antibodies were obtained by design. In addition, the 5′ end of the heavy chain and the light chain were respectively added with a nucleotide sequence encoding a signal peptide optimized according to the amino acid sequence; in addition, a stop code was added to the 3′ end of the light chain and the heavy chain nucleotide sequence, respectively.

### EXAMPLE 2 GENE SYNTHESIS AND CONSTRUCTION OF EXPRESSION VECTORS

pcDNA3.1-G418 vector was used as a plasmid vector for the expression of the multifunctional antibody. pcDNA3.1-G418 vector contained the promoter CMVPromoter, the eukaryotic screening marker G418 tag, and the prokaryotic screening tag Ampicilline. Nucleotide sequences for the expression of the light chain and the heavy chain of the constructed antibody were obtained by gene synthesis, and the vector and the target fragment were double-digested with HindIII and XhoI, and then enzyme-linked by DNA ligase after recovery, and transformed into E. coli competent cell DH5α. Positive clones were selected and plasmid extraction and enzyme digestion verification were performed to obtain the plasmid containing said antibody.

### EXAMPLE 3 PLASMID EXTRACTION

The recombinant plasmids containing each of the above-mentioned target genes were transformed into E. coli competent cell DH5α, and the transformed bacteria were coated on LB plates containing 100 μg/mL ampicillin for incubation, and the plasmid clones were selected into liquid LB medium for incubation, and shaken at 260 rpm for 14 hours. The plasmids were extracted by the endotoxin-free plasmid large extraction kit, and dissolved in sterile water, and the concentration was determined with a nucleic acid protein quantifier.

### EXAMPLE 4 PLASMID TRANSFECTION, TRANSIENT EXPRESSION AND ANTIBODY PURIFICATION

ExpiCHO was cultured to a cell density of 6×10⁶ cells/mL at 37° C., 8% CO₂, and 100 rpm. The constructed plasmids were transfected into the above cells by liposomes according to combination pairs. The concentration of the transfected plasmids was 1 mg/mL, and the volume of the liposomes was determined by reference to the ExpiCHO™ Expression System kit, and cultured at 32° C., 5% CO₂, and 100 rpm for 7-10 days. Feeding was given once after 18-22 h of transfection and once between day 5, respectively. The above culture product was centrifuged at 4000 g, and filtered through a 0.22 μm filter membrane and the supernatant of the medium was collected. The antibody expression in the supernatant was detected, and the results are shown in Table 3. The antibody proteins obtained were purified by Protein A and ionic column, and the eluent was collected.

The specific operation steps for Protein A and ionic column purification were as follows: cell culture fluid was centrifuged at high speed and the supernatant was taken, and affinity chromatography was performed using GE's Protein A chromatography column. The equilibrium buffer used for chromatography was 1×PBS (pH 7.4). After the cell supernatant was loaded and combined, it was washed with PBS until the ultraviolet rays returned to the baseline, and then the target protein was eluted with the elution buffer 0.1 M glycine (pH 3.0), and then the pH was adjusted to neutral using Tris for storage. The product from affinity chromatography was adjusted to pH of 1-2 pH units below or above pI, and appropriately diluted to control the sample conductance below 5 ms/cm. Appropriate corresponding pH buffers such as phosphate buffer, acetate buffer and other conditions, and conventional ion exchange chromatography methods in the field such as anion exchange or cation exchange were used to carry out NaCl gradient elution under the corresponding pH conditions, and the collection tubes where the target proteins were located were selected and combined for storage according to SDS-PAGE.

Then, the eluent obtained after purification was ultrafiltrated into the buffer. Proteins were detected by SDS-polyacrylamide gel electrophoresis assay.

SDS-PAGE analysis showed that the non-reducing gel condition contained the target bands, and the target antibodies under the reducing gel condition all contained the target bands, corresponding to the heavy chain and light chain of the desired antibody. Therefore, the structurally correct antibodies were proved to be obtained by plasmid transfection, transient expression and antibody purification.

TABLE 3
Transient expression of constructed antibodies

| Structure code | Expression (mg/mL) |
|---|---|
| CNVR-A | 0.211 |
| CNVR-B | 0.095 |
| CNVR-C | 0.095 |
| CNV-A1 | 0.061 |
| CNV-B1 | 0.062 |
| CNV-C1 | 0.054 |
| CNV-A2 | 0.065 |
| CNV-B2 | 0.053 |
| CNV-C2 | 0.051 |

### EXAMPLE 5 DETECTION OF THE AFFINITY OF THE ANTIBODIES TO NKP30 BY ELISA

Human-NKP30-His was diluted to 0.5 μg/mL with PBS buffer at pH 7.4, added into the 96-well ELISA plate at 100 μL per well, coated overnight at 4° C., and blocked with 1% BSA blocking solution for 1 hour. The plate was washed 3 times with PBST, and the constructed antibodies were diluted to 10 μg/mL with 0.5% BSA sample diluent as the starting concentration, and then diluted by 3-fold gradient (11 gradients in total). A negative control (blank well and IgG1 isotype control) and a positive control were set, and the positive control was a NKP30 humanized antibody (sequence shown in SEQ ID No.32), and incubated at 37° C. for 1 h at 100 μL per well. The plate was washed 3 times with PBST, and then HRP-labelled goat anti-human IgG-Fc was diluted with the sample diluent at 1:20000. 100 μL was added into each well, and incubated at room temperature for 1 hour. The plate was washed 4 times with PBST, and 100 μL of TMB substrate was added into each well, and incubated at room temperature without light for 10 minutes. 100 μL of 1 M HCl solution was added into each well to terminate the color development reaction. The absorbance value of each well in the 96-well plate was determined by selecting the wavelength of 450 nm and the reference wavelength of 570 nm on a multifunctional enzyme marker, and the absorbance value (OD)=OD₄₅₀ₙₘ−OD₅₇₀ₙₘ. The logarithm of the concentration of the constructed antibodies were taken as the abscissa, and the measured absorbance value of each well was taken as the ordinate, and the Sigmoidaldose-response (Variable Slope) method (GraphPad Prism software, GraphPad Software, San Diego, California) was selected for nonlinear regression to obtain the binding curve of the target antibody to NKP30 protein.

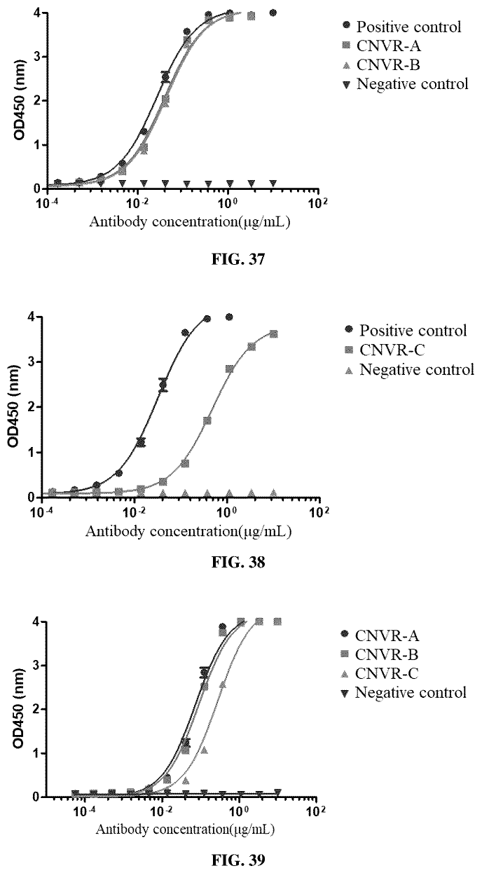

The ELISA results of the constructed antibody molecule were shown in FIGS. 37-38. All structures of the multifunctional antibody can bind to NKp30 at all concentrations. Compared with the positive control, CNVR-A and CNVR-B have no obvious difference in the binding activity with NKp30 protein.

### EXAMPLE 6 DETECTION OF THE AFFINITY OF VEGFR1 END OF THE ANTIBODIES TO VEGFA BY ELISA

Human VEGFA-his protein (Acro, Cat: VE5-H5248) was diluted to 1 μg/mL with PBS buffer at pH 7.4, added into the 96-well ELISA plate at 100 μL per well, coated overnight at 4° C., and blocked with 1% BSA blocking solution for 1 hour. The plate was washed 3 times with PBST, and the constructed expressing antibodies were diluted to 20 μg/mL with 0.5% BSA sample diluent as the starting concentration, and then diluted by 3-fold gradient (11 gradients in total). A negative control (blank well and IgG1 isotype control) was set and incubated at 37° C. for 1 h at 100 μL per well. The plate was washed 3 times with PBST, and then HRP-labelled goat anti-human IgG-Fc was diluted with the sample diluent at 1:20000. 100 μL was added into each well, and incubated at room temperature for 1 hour. The plate was washed 4 times with PBST, and 100 μL of TMB substrate was added into each well, and incubated at room temperature without light for 10 minutes. 100 μL of 1 M HCl solution was added into each well to terminate the color development reaction. The absorbance value of each well in the 96-well plate was determined by selecting the wavelength of 450 nm and the reference wavelength of 570 nm on a multifunctional enzyme marker, and the absorbance value (OD)=OD₄₅₀ₙₘ−OD₅₇₀ₙₘ. The logarithm of the concentration of the constructed antibodies were taken as the abscissa, and the measured absorbance value of each well was taken as the ordinate, and the Sigmoidaldose-response (Variable Slope) method (GraphPad Prism software, GraphPad Software, San Diego, California) was selected for nonlinear regression to obtain the binding curve of the target antibodies to the VEGFA protein.

The ELISA results of the constructed antibody molecules were shown in FIG. 39, respectively. The three multifunctional antibodies can bind to VEGFA at all concentrations, while the negative control did not bind to VEGFA. In general, all the constructed antibodies showed binding activity with VEGFA protein.

### EXAMPLE 7 DETECTION OF THE BINDING OF THE CONSTRUCTED ANTIBODIES TO PANC-1-CLAUDIN18.2 CELLS BY FLOW CYTOMETRY

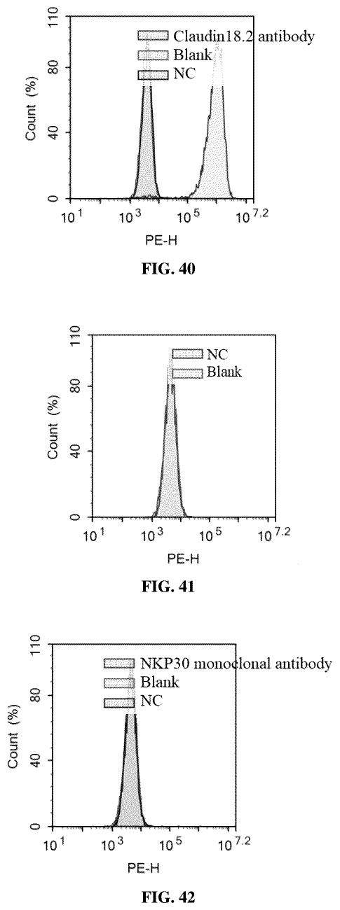

PANC-1-Claudin18.2 was an artificially constructed Claudin18.2 overexpressing PANC-1 engineered cell line, which has been validated by Claudin18.2 monoclonal antibody (consisting of SEQ ID No.22 and SEQ ID No.33), as shown in FIG. 40. PANC-1-Claudin18.2 cell suspension with cells in good condition and in logarithmic phase of growth was taken, 800 g was centrifuged for 3 minutes at room temperature, the supernatant was removed, cells were washed twice with PBS and then resuspended with PBS to 2×10⁵/mL and divided into portions according to 1 mL/centrifuge tube. Added 100 μL PBS diluted to 10 μg/mL of the constructed antibodies, that was, detected the primary antibody, and set up a blank control group (no primary antibody and fluorescent secondary antibody), a negative control group (no primary antibody), an isotype control group (IgG1), the negative control is NKP30 monoclonal antibody (SEQ ID No.32), were mixed thoroughly and incubated for half an hour at room temperature. 800 g was centrifuged for 5 minutes at room temperature, the supernatant containing antibody was removed, and washed 3 times with PBS. 100 μL of resuspended cells was taken, added 0.2 L PE-labelled PE anti-human IgG Fc (Biolegend Cat: 398004) fluorescent secondary antibody, mixed well and incubated at room temperature away from light for 30 min; 800 g was taken to centrifuge for 5 minutes at room temperature, removed the supernatant containing the secondary antibody, and washed the cells 3 times with PBS; cells were resuspended with 100 μL PBS for flow cytometry.

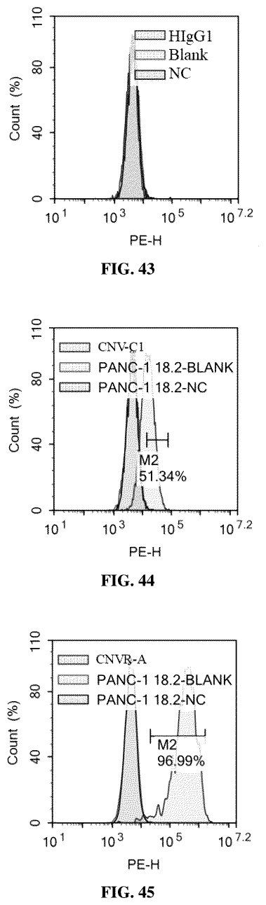

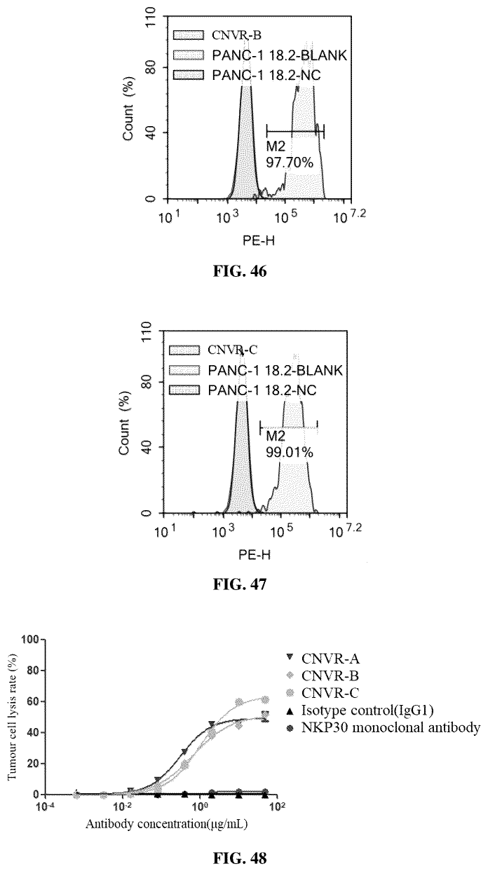

The results were shown in FIGS. 41-43, compared with the blank control group, the IgG1, NKp30 monoclonal antibody and negative control had no binding to PANC-1-claudin 18.2 cells, while the constructed antibodies (as shown in FIGS. 44-47) had good binding to PANC-1-claudin 18.2 cells.

### EXAMPLE 8 THE CONSTRUCTED ANTIBODY-MEDIATED PANC-1-CLAUDIN18.2 CELL KILLING EXPERIMENT

The constructed antibodies CNVR-A, CNVR-B and CNVR-C were selected to perform the specific killing experiment on PANC-1-Claudin18.2 tumor cells. PANC-1-Claudin18.2 cells with normal morphology and logarithmic phase were used, and after digestion by trypsin, neutralized with complete medium, centrifuged at 1000 rpm at room temperature for 4 min and resuspended with RPMI 1640 base medium (containing 5% FBS), and then spreaded on 96-well plates at 2×10⁴/well and 50 L/well. The constructed antibodies were diluted to 50 nM using RPMI 1640 base medium (containing 5% FBS), and then diluted by 5-fold gradient (8 gradients in total) at 100 μL/well, and 3 replicates were set. NK cells were resuspended and added into the corresponding wells at 6×10⁴/well and 50 μL/well to make the efficiency target ratio 3:1. At the same time, the maximum target cell lysis well (M), target cell spontaneous release well (ST), effector cell spontaneous release well (SE), total volume correction blank well (BV) and medium blank control well (BM) were set up. After standing for 10 min, it was centrifuged at 1000 rpm at room temperature for 4 min, and incubated in 5% CO₂ and 37° C. carbon dioxide cell incubator for 4 h. Lysate was added into the M and B-V wells 45 min in advance, mixed well, and centrifuged at 1000 rpm at room temperature for 4 min at the end of incubation. 50 μL supernatant was absorbed into the LDH assay plate, and then the substrate dissolved in assay buffer was added at 50 μL/well and reacted at room temperature without light for 30 min. Then termination solution was added at 50 μL/well, and read at 490 nm (Cyto Tox96 Non-Radioactive Cytotoxicity Assay, Cat: G₁₇₈₀) after standing for 10 min. The cell lysis rate was calculated as the formula OD (sample well, ST, SE)-OD (B-M), OD (M)-OD (B-V), Lysis %=OD (sample well-ST-SE)×100/OD (M-ST), and the relationship between Lysis % and concentration was plotted using GraphPad Prism software.

As can be seen from FIG. 48, PANC-1-Claudin18.2 cells in the constructed antibody group lysed and died, while those in the irrelevant antibody group had no significant anti-tumor activity, and NKp30 monoclonal antibody also had no anti-tumor activity, indicating that the constructed antibodies mediated NK cells to specifically kill Claudin18.2 positive PANC-1-Claudin 18.2 target cells.

The protection content of the present invention is not limited to the above embodiments. Without departing from the spirit and scope of the inventive concept, variations and advantages that can be thought of by those skilled in the art are included in the present invention and are protected by the appended claims.

1. A multispecific antigen-binding protein, comprising:
(a) a first antigen-binding portion that recognizes the first antigen, wherein the first antigen is targeted to antigens associated with tumorigenesis or progression;
(b) a second antigen-binding portion, wherein the second antigen-binding portion is an innate immune cell agonist; and
(c) a third antigen-binding portion that recognizes the third antigen, wherein the third antigen regulates tumor microenvironment by regulating normal and abnormal angiogenesis;
wherein the positions of the second antigen-binding portion and the third antigen-binding portion are interchangeable.

2. The multispecific antigen-binding protein according to claim 1, wherein the antigen targeting the tumorigenesis or progression is a tumor-associated antigen (TAA) and/or a tumor-specific antigen (TSA).

3. The multispecific antigen-binding protein according to claim 1, wherein the innate immune cell agonist is an NK cell agonist and/or a γδT cell agonist.

4. The multispecific antigen-binding protein according to claim 3, wherein the second antigen-binding protein recognizes the second antigen expressed on the innate immune cells, and the second antigen-binding protein activates the innate immune cells after binding to the second antigen.

5. The multispecific antigen-binding protein according to claim 3, wherein the third antigen is a pro-angiogenic factor.

6. The multispecific antigen-binding protein according to claim 5, wherein the first antigen-binding portion and/or the second antigen-binding portion and/or the third antigen-binding portion is a full-length antibody comprising two heavy chains and two light chains.

7. The multispecific antigen-binding protein according to claim 5, wherein the first antigen-binding portion and/or the second antigen-binding portion and/or the third antigen-binding portion is an antibody fragment comprising a heavy chain variable domain (VH) and/or a light chain variable domain (VL).

8. The multispecific antigen-binding protein according to claim 1, wherein the first antigen-binding portion and/or the second antigen-binding portion and/or the third antigen-binding portion is one of Fab, scFab, F(ab′)2, Fv, dsFv, scFv, VH or VL structural domain.

9. The multispecific antigen-binding protein according to claim 5, wherein the first antigen-binding portion and/or the second antigen-binding portion and/or the third antigen-binding portion is a single-domain antibody (VHH).

10. The multispecific antigen-binding protein according to claim 5, wherein the first antigen-binding portion and/or the second antigen-binding portion and/or the third antigen-binding portion is a receptor for an antigen.

11. The multispecific antigen-binding protein according to claim 5, wherein the second antigen-binding portion is fused to the N-terminus and/or C-terminus of one of the heavy chains of the first antigen-binding portion.

12. (canceled)

13. (canceled)

14. (canceled)

15. (canceled)

16. The multispecific antigen-binding protein according to claim 5, wherein the second antigen-binding portion is fused to the N-terminus and/or C-terminus of one of the light chains of the first antigen-binding portion.

17. (canceled)

18. (canceled)

19. (canceled)

20. (canceled)

21. The multispecific antigen-binding protein according to claim 5, wherein the second antigen-binding portion is fused to the N-terminus and/or C-terminus of both heavy chains of the first antigen-binding portion.

22. (canceled)

23. (canceled)

24. (canceled)

25. The multispecific antigen-binding protein according to claim 5, wherein the second antigen-binding portion is fused to the N-terminus and/or C-terminus of both light chains of the first antigen-binding portion.

26. (canceled)

27. (canceled)

28. (canceled)

29. The multispecific antigen-binding protein according to claim 5, wherein the third antigen-binding portion replaces one or more binding units of: Fab region or Fv region or VH, CH1 domain, CH2 domain, or CH3 domain of the first antigen-binding portion, and/or the second antigen-binding portion.

30. The multispecific antigen-binding protein according to claim 5, wherein the third antigen-binding portion is located between any one of: VH, CH1 domain, CH2 domain, and CH3 domain of the first antigen-binding portion and/or the second antigen-binding portion.

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. The multispecific antigen-binding protein according to claim 5, wherein the tumor-associated antigen is selected from the group consisting of: GPC3, CD19, CD20 (MS4A1), CD22, CD24, CD30, CD33, CD38, CD40, CD123, CD133, CD138, CDK4, CEA, Claudin6, Claudin18.2, CCR8, AFP, ALK, B7H3, BAGE protein, BCMA, BIRC5 (survivin), BIRC7, β-catenin, brc-ab1, BRCA1, BORIS, CA9, CA125, carbonic anhydrase IX, caspase-8, CALR, CCR5, NA17, NKG2D, NY-BR1, NY-BR62, NY-BR85, NY-ESO1, OX40, p15, p53, PAP, PAX3, PAX5, PCTA-1, PLAC1, PRLR, PRAME, PSMA (FOLH1), RAGE proteins, cyclin-B1, CYPIB1, EGFR, EGFRVIII, ErbB2/Her2, ErbB3, ErbB4, ETV6-AML, EpCAM, EphA2, Fra-1, FOLR1, GAGE protein, GD2, GD3, GloboH, GM3, gp100, Her2, HLA/B-raf, HLA/k-ras, HLA/MAGE-A3, hTERT, IL13Rα2, LMP2, κ-Light, LeY, MAGE-1, MAGE-2, MAGE-3, MAGE-4, MAGE-6, MAGE-12, MART-1, mesothelin, ML-IAP, MOv-γ, Muc1, Muc2, Muc3, Muc4, Muc5, Muc16, MUM1, Ras, RGS5, Rho, ROR1, SART-1, SART-3, STEAP1, STEAP2, TAG-72, TGF-β, TNFR2, TMPRSS2, Thom-Knott's antigen, TRP-1, TRP-2, tyrosinase and urolytic protein-3, 5T4, hepatitis B surface antigen (HBSAG), tissue polypeptide antigen (TPA), carbohydrate antigen 153 (CA153), carbohydrate antigen 19-9 (CA19-9), carbohydrate antigen 724 (CA724), carbohydrate antigen 242 (CA242), carbohydrate antigen 50 (CA50), CYFRA21-1 (Cy211), neuron specific enolase (NSE), prostate-specific antigen (PSA), human chorionic gonadotropin (HCG), thyroglobulin (TG), ferritin (SF), B2-microglobulin (B2-MG), squamous cell antigen (SCC).

49. The multispecific antigen-binding protein according to claim 5, wherein the second antigen is selected from the group consisting of: NKP30, NKP46, CD16, NKP44, CD244, CD226, NKG2E, NKG2D, NKG2C, and KIR.

50. The multispecific antigen-binding protein according to claim 5, wherein the second antigen is selected from the group consisting of: Vδ1T, Vδ2T, Vδ3T, γδTreg, γδT17, IFN-γ⁺γδT.

51. The multispecific antigen-binding protein according to claim 5, wherein the third antigen is selected from the group consisting of: EGF, FGF, FGF, FGF-α, FGF-β, VEGF, VEGFR, HDGF, HGF, HGFK1, NRP-1, ANG, Ang1, Ang2, PDGF, PDGFR, PIGF, TGF-α, TGF-β, TGF-β receptor, CD31, CD105, MCP-1, COX-2, AC133, Id2/Id3, IL-1α, IL-6, IL-1β, IL-8, CXCL5, MMP, and THBS, AREG, ET-1, AAMP, AGGF1, AMOT, ANGLPTL3, ANGPTLA, BTG1, NOS3, TNFSF12, VASH2, integrinαvβ3, α5β1, VE-cadherin, leptin, heparin, plasminogen activator, and plasminogen activator inhibitor-1.

52. (canceled)

53. (canceled)

54. (canceled)

55. (canceled)

56. (canceled)

57. (canceled)

58. (canceled)

59. (canceled)

60. (canceled)

61. (canceled)
